(19) United States
(12) Patent Application Publication
(10) Pub. No.: US 2024/0141199 A1
(43) Pub. Date: May 2, 2024

(54) CHEMICAL COMPOUND, ELECTROOPTIC COMPOSITION, ELECTROOPTIC FILM, AND ELECTROOPTIC ELEMENT

(71) Applicant: Sumitomo Chemical Company, Limited, Chuo-ku, Tokyo (JP)

(72) Inventors: Eiji YOSHIKAWA, Osaka-shi, Osaka (JP); Takayuki OKACHI, Tsukuba-shi, Ibaraki (JP); Yoshiaki TSUBATA, Tsukuba-shi, Ibaraki (JP); Takahiro SATO, Tsukuba-shi, Ibaraki (JP)

(21) Appl. No.: 18/267,877

(22) PCT Filed: Dec. 14, 2021
(86) PCT No.: PCT/JP2021/045956

§ 371 Date: Jun. 16, 2023

(30) Foreign Application Priority Data
Dec. 18, 2020 (JP) 2020-210543

(51) Int. Cl. C09D 151/00; C07D 495/04; C07D 495/14; C07F 7/18

(52) U.S. Cl. CPC C09D 151/006; C07D 495/04; C07D 495/14; C07F 7/1804

(57) ABSTRACT

A compound having formula (1) has excellent heat resistance.

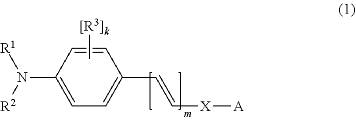

In formula (1), X represents a divalent polycyclic condensed ring group having two or more thiophene rings and having an sp3 carbon atom as a constituent element. R¹ and R² each represent an alkyl group, R³ represents an alkyl group, k represents an integer of 0 to 4, and A represents a group having formula (a1), (a2), or (b1):

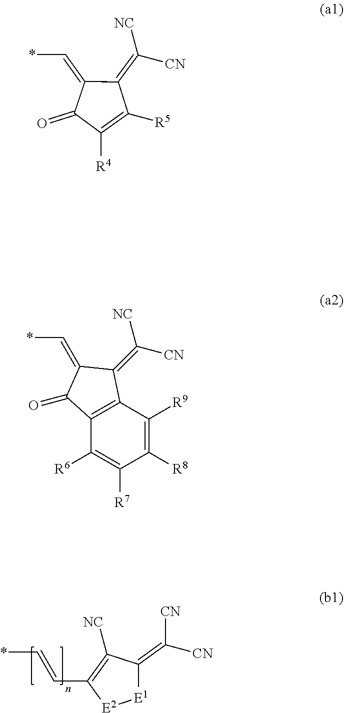

R⁴, R⁵, R⁶, R⁷, R⁸, and R⁹ each represent an alkyl group, E¹ and E² each represent —C(R¹⁰)(R¹¹)—, —C(O)—, —O—, or —NR¹²—. R¹⁰ and R¹¹ each represent an alkyl group, R¹² represents an alkyl group, m and n each represent 0 or 1, where m+n=1.

### TECHNICAL FIELD

The present invention relates to a compound, an electrooptic composition, an electrooptic film, and an electrooptic element.

### BACKGROUND ART

As an electrooptic (hereinafter, also abbreviated as “EO”) material applicable to a light control element (optical element) such as an optical modulator, an optical switch, an optical interconnect, an optoelectronic circuit, wavelength conversion, an electric field sensor, terahertz (THz) wave generation and detection, or an optical phased array, an inorganic ferroelectric EO material is conventionally used. However, the inorganic ferroelectric EO material has limitations in terms of high speed, miniaturization, and integration. Therefore, in order to achieve next-generation ultra-high-speed optical communication, a material capable of high-speed operation and capable of hybrid with silicon photonics is required.

An organic EO material has attracted attention from such a viewpoint. The organic EO material exhibits a large electrooptical effect as compared with the inorganic ferroelectric EO material, can operate at high speed, and can be miniaturized and integrated by hybrid with silicon photonics, and thus is expected as a material that performs next-generation optical communication.

A compound used for the organic EO material (hereinafter, also referred to as “EO compound”) has a structure in which a donor and an acceptor are linked to each other by a n conjugated bridge as a basic structure. In order to increase an EO coefficient of the EO material, it is known to adopt a donor having a high electron donating property and an acceptor having a high electron withdrawing property for the EO compound to increase the length of the n conjugated bridge. As the EO compound having such a structure, EO compounds having various structures have been reported (for example, Patent Document 1 and Non-Patent Document 1).

By the way, when an EO element in which an optical waveguide is formed of an EO material is produced, an EO compound may be subjected to an orientation treatment in order to generate secondary EO activity of the EO material. As a method for subjecting an EO compound to an orientation treatment, an electric field poling method is generally used. The electric field poling method is a method in which an electric field is applied to an EO material, and an EO compound is oriented in a direction of the applied electric field by a Coulomb force between dipole moment of the EO compound and the applied electric field.

In such an electric field poling method, an electric field is usually applied in a state where heating is performed to a temperature near a glass transition temperature of a host material to promote a molecular motion of the EO compound. Therefore, in order to obtain an EO element that exhibits excellent EO performance, in addition to having excellent EO characteristics, the EO compound requires to have such heat resistance that the EO compound is not deteriorated by heating in an orientation treatment.

Furthermore, in response to a demand for high speed of an electronic circuit, efforts have been made to improve a speed of signal transmission by connecting electronic circuits to each other with an optical circuit, and it has been studied to use an EO element using an EO material for conversion of an electrical signal and an optical signal. At this time, since an electronic circuit operating at a high speed has a high temperature, a molecular motion of a nonlinear optical compound may be active, and orientation may be relaxed. For this reason, the glass transition temperature of the host material requires a higher temperature, which in turn requires heat resistance (thermal stability) of the EO compound at a higher temperature.

### PRIOR ART DOCUMENTS

### Patent Document

Patent Document 1: JP-A-2004-501159

### Non-Patent Document

Non-Patent Document 1: Chem. Mater. 2008, 120, 6372-6377.

### SUMMARY OF THE INVENTION

### Problems to be Solved by the Invention

However, a conventional EO compound does not have sufficient heat resistance (thermal stability), and tends to decompose under high temperature conditions of a certain level or higher. When the heat resistance of the EO compound is insufficient, there is a possibility that processing process conditions such as element formation are limited.

Therefore, an object of the present invention is to provide a compound having excellent heat resistance. Another object of the present invention is to provide an electrooptic composition, an electrooptic film, and an electrooptic element using such a compound.

### Means for Solving the Problems

As a result of intensive studies in view of the above problems, the present inventors have found that an EO compound having a high hyperpolarizability but excellent heat resistance is provided by introducing a predetermined polycyclic condensed ring group and a predetermined linking group linking the polycyclic condensed ring group into a n-conjugated bridge in a donor/n-conjugated bridge/acceptor structure, and have completed the present invention.

The present invention provides compounds according to [1] to [3], an electrooptic composition according to [4], an electrooptic film according to [5], and an electrooptic element according to [6] described below.
[1] A compound represented by the following formula (1).

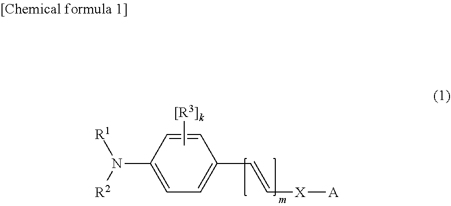

[In formula (1), X represents a divalent polycyclic condensed ring group having two or more thiophene rings and having at least one selected from the group consisting of an sp3 carbon atom, a nitrogen atom, and a silicon atom as a constituent element, and the divalent polycyclic condensed ring group may have a substituent.

R¹ and R² each independently represent an alkyl group, a haloalkyl group, an acyloxyalkyl group, a trialkylsilyloxyalkyl group, an aryldialkylsilyloxyalkyl group, an alkyldiarylsilyloxyalkyl group, an aryl group, —R²¹—OH (R²¹ represents a divalent hydrocarbon group), —R²²—NH₂ (R²² represents a divalent hydrocarbon group), —R²³—SH (R²³ represents a divalent hydrocarbon group), or —R²⁴—NCO (R²⁴ represents a divalent hydrocarbon group). These groups may each have a crosslinkable group. R¹ and R² may be bonded to each other to form a ring together with atoms to which R¹ and R² are bonded.

R³ represents an alkyl group, an alkyloxy group, an aryl group, an aryloxy group, an aralkyloxy group, a trialkylsilyloxyalkyl group, an aryldialkylsilyloxyalkyl group, an alkyldiarylsilyloxyalkyl group, an alkenyloxy group, an alkynyloxy group, a hydroxy group, an amino group, a sulfanyl group, an isocyanate group, —R³¹—OH (R³¹ represents a divalent hydrocarbon group), —O—R³²—OH (R³² represents a divalent hydrocarbon group), —R³³—NH₂ (R³³ represents a divalent hydrocarbon group), —R³⁴—SH (R³⁴ represents a divalent hydrocarbon group), —R³⁵—NCO (R³⁵ represents a divalent hydrocarbon group), or —OC(═O)R⁴¹ (R⁴¹ represents a monovalent hydrocarbon group). These groups may each have a crosslinkable group. When there is a plurality of R³s, R³s may be the same as or different from each other. R³ may be bonded to R¹ or R² to form a ring together with atoms to which R³ and R¹ or R² are bonded.

k represents an integer of 0 to 4.

A represents any one of groups represented by the following formulas (a1), (a2), and (b1).

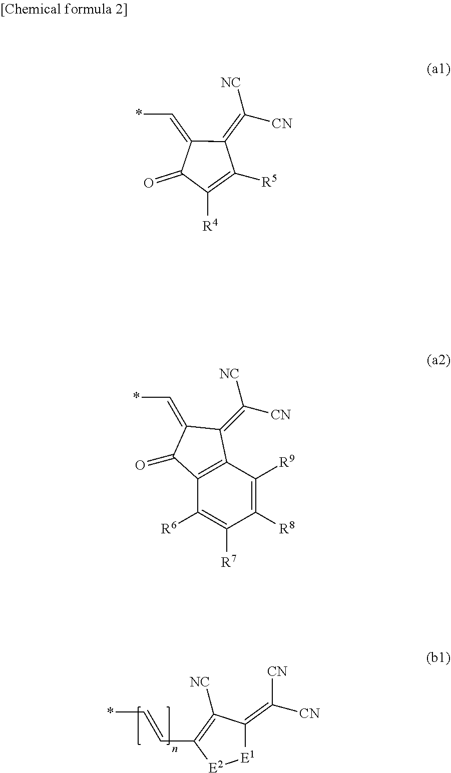

R⁴, R⁵, R⁶, R⁷, R⁸, and R⁹ each independently represent a hydrogen atom, a halogen atom, an alkyl group, a haloalkyl group, a cyano group, an aryl group, or a haloaryl group.

E¹ and E² each independently represent —C(R¹⁰)(R¹¹)—, —C(O)—, —O—, or —NR¹²—. Note that at least one of E¹ and E² is —O— or —NR¹²—. R¹⁰ and R¹¹ each independently represent a hydrogen atom, an alkyl group, a haloalkyl group, an aryl group, or a haloaryl group. R¹² represents a hydrogen atom or an alkyl group.

When A is a group represented by formula (a1) or (a2), m is 0.

When A is a group represented by formula (b1), m and n each independently represent 0 or 1. Note that m+n=1.]
[2] The compound according to [1], in which the compound represented by the above formula (1) is a compound represented by the following formula (2a2) or (2b1).

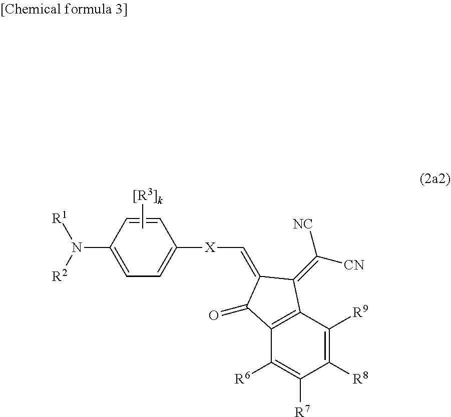

[In formula (2a2), X, R¹, R², R³, R⁶, R⁷, R¹, R⁹, and k have the same meanings as those described above.]

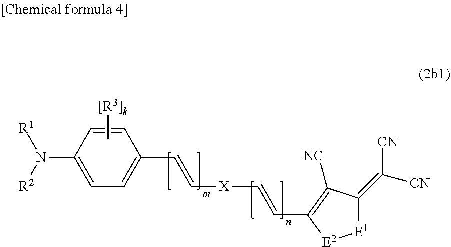

[In formula (2b1), X, R¹, R², R³, E¹, E², k, m, and n have the same meanings as those described above.]
[3] The compound according to [1] or [2], in which the compound represented by the above formula (1) is a compound represented by the following formula (2b1-1).

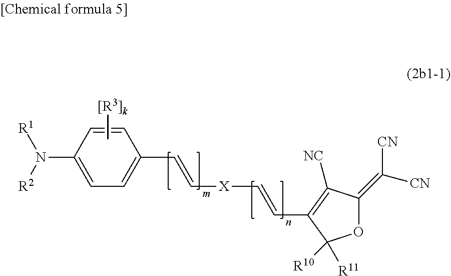

[In formula (2b1-1), X, R¹, R², R³, k, m, and n have the same meanings as those described above.

R¹⁰ and R¹¹ each independently represent a methyl group, a trifluoromethyl group, a phenyl group, or a pentafluorophenyl group.]
[4] An electrooptic composition containing the compound according to any one of [1] to [3].
[5] An electrooptic film containing the compound according to any one of [1] to [3].
[6] An electrooptic element including the electrooptic film according to [5].

In addition, the present invention relates to use of the compound according to any one of [1] to [3] as an electrooptic material. Furthermore, the present invention relates to use of a composition containing the compound according to any one of [1] to [3] as an electrooptic material.

### Effect of the Invention

The present invention provides a compound having excellent heat resistance. In addition, the compound has a high hyperpolarizability. Therefore, by using the compound in production of an EO element, heat resistance required for a high-temperature process at the time of poling, a thermal curing process for fixing film orientation, a high-temperature process at the time of mounting, and the like can be improved, and the degree of freedom in an element production process can be increased. In addition, the present invention provides an electrooptic composition, an electrooptic film, and an electrooptic element using such a compound.

### MODE FOR CARRYING OUT THE INVENTION

Hereinafter, a preferred embodiment of the present embodiment will be described in detail.

### [Compound]

A compound of the present embodiment is a compound represented by the following formula (1). The compound represented by formula (1) has a predetermined polycyclic condensed ring group and a predetermined linking group linking the polycyclic condensed ring group. By having a predetermined polycyclic condensed ring group, the compound represented by formula (1) has a high hyperpolarizability. In addition, by having a predetermined polycyclic condensed ring group and a predetermined linking group linking the polycyclic condensed ring group, the compound represented by formula (1) can suppress a multimerization reaction (for example, a Diels-Alder reaction) between the molecules by heating, and has excellent heat resistance.

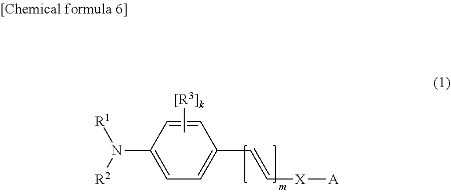

In formula (1), X represents a divalent polycyclic condensed ring group having two or more thiophene rings and having at least one selected from the group consisting of an sp3 carbon atom, a nitrogen atom, and a silicon atom as a constituent element. The divalent polycyclic condensed ring group may have a substituent.

The divalent polycyclic condensed ring group as X has two or more thiophene rings. The number of thiophene rings is preferably 2 to 10, more preferably 2 to 8, and still more preferably 2 to 6. Note that, in a ring-condensed thiophene in which molecules of thiophene are ring-condensed, the number of ring-condensed molecules of thiophene is the number of thiophene rings. For example, in thienothiophene in which two molecules of thiophene are ring-condensed, the number of thiophene rings is 2.

The divalent polycyclic condensed ring group as X has at least one selected from the group consisting of an sp3 carbon atom, a nitrogen atom, and a silicon atom as a constituent element. That is, the divalent polycyclic condensed ring group has at least one group selected from the group consisting of a group represented by —C(R^{A})(R^{B})—in the ring, a group represented by —N(R^{C})— in the ring, and a group represented by —Si(R^{D})(R^{E})— in the ring. A carbon atom in —C(R^{A})(R^{B})— may be a tertiary carbon atom in which one of R^{A} and R^{B} is an alkyl group or the like and the other is a hydrogen atom, or a quaternary carbon atom in which both R^{A} and R^{B} are alkyl groups and the like, and is preferably a quaternary carbon atom.

The number of sp3 carbon atoms in the divalent polycyclic condensed ring group as X is preferably 1 to 6, more preferably 1 to 4, and still more preferably 1 or 2. The number of nitrogen atoms in the divalent polycyclic condensed ring group as X is preferably 1 to 6, more preferably 1 to 4, and still more preferably 1 or 2. The number of silicon atoms in the divalent polycyclic condensed ring group as X is preferably 1 to 6, more preferably 1 to 4, and still more preferably 1 or 2.

R^{A}, R^{B}, R^{C}, R^{D}, and R^{E} each independently represent a hydrogen atom, an alkyl group, a cycloalkyl group, an alkyloxy group, a cycloalkyloxy group, an alkylthio group, a cycloalkylthio group, an aryl group, or a monovalent heterocyclic group. These groups may each have a substituent.

The alkyl group as each of R^{A}, R^{B}, R^{C}, R^{D}, and R^{E} may be linear or branched. The number of carbon atoms of the alkyl group is usually 1 to 30 without including the number of carbon atoms of a substituent. Specific examples of the alkyl group include a methyl group, an ethyl group, a propyl group, an isopropyl group, a butyl group, an isobutyl group, a sec-butyl group, a tert-butyl group, a pentyl group, an isopentyl group, a 2-methylbutyl group, a 1-methylbutyl group, a hexyl group, an isohexyl group, a 3-methylpentyl group, a 2-methylpentyl group, a 1-methylpentyl group, a heptyl group, an octyl group, an isooctyl group, a 2-ethylhexyl group, a 3,7-dimethyloctyl group, a nonyl group, a decyl group, an undecyl group, a dodecyl group, a tetradecyl group, a hexadecyl group, an octadecyl group, and an eicosyl group.

The number of carbon atoms of the cycloalkyl group as each of R^{A}, R^{B}, R^{C}, R^{D}, and R^{E} is usually 3 to 30 without including the number of carbon atoms of a substituent. Specific examples of the cycloalkyl group include a cyclopropyl group, a cyclopentyl group, a cyclohexyl group, and an adamantyl group.

The alkyl group in the alkyloxy group as each of R^{A}, R^{B}, R^{C}, R^{D}, and R^{E} may be linear or branched. The number of carbon atoms of the alkyloxy group is usually 1 to 30 without including the number of carbon atoms of a substituent. Specific examples of the alkyloxy group include a methoxy group, an ethoxy group, a propoxy group, an isopropoxy group, a butoxy group, an isobutoxy group, a tert-butoxy group, a pentyloxy group, a hexyloxy group, a heptyloxy group, an octyloxy group, a 2-ethylhexyloxy group, a nonyloxy group, a decyloxy group, a 3,7-dimethyloctyloxy group, and a lauryloxy group.

The number of carbon atoms of the cycloalkyloxy group as each of R^{A}, R^{B}, R^{C}, R^{D}, and R^{E} is usually 3 to 30 without including the number of carbon atoms of a substituent. Specific examples of the cycloalkyloxy group include a cyclopropyloxy group, a cyclopentyloxy group, a cyclohexyloxy group, and an adamantyloxy group.

The alkyl group in the alkylthio group as each of R^{A}, R^{B}, R^{C}, R^{D}, and R^{E} may be linear or branched. The number of carbon atoms of the alkylthio group is usually 1 to 30 without including the number of carbon atoms of a substituent. Specific examples of the alkylthio group include a methylthio group, an ethylthio group, a propylthio group, an isopropylthio group, a butylthio group, an isobutylthio group, a tert-butylthio group, a pentylthio group, a hexylthio group, a heptylthio group, an octylthio group, a 2-ethylhexylthio group, a nonylthio group, a decylthio group, a 3,7-dimethyloctylthio group, and a laurylthio group.

The number of carbon atoms of the cycloalkylthio group as each of R^{A}, R^{B}, R^{C}, R^{D}, and R^{E} is usually 3 to 30 without including the number of carbon atoms of a substituent. Specific examples of the cycloalkylthio group include a cyclopropylthio group, a cyclopentylthio group, a cyclohexylthio group, and an adamantylthio group.

The number of carbon atoms of the aryl group as each of R^{A}, R^{B}, R^{C}, R^{D}, and R^{E} is usually 6 to 30 without including the number of carbon atoms of a substituent. Specific examples of the aryl group include a phenyl group, a 1-naphthyl group, a 2-naphthyl group, a 1-anthracenyl group, a 2-anthracenyl group, a 9-anthracenyl group, a 1-pyrenyl group, a 2-pyrenyl group, a 4-pyrenyl group, a 2-fluorenyl group, a 3-fluorenyl group, a 4-fluorenyl group, a 2-phenylphenyl group, a 3-phenylphenyl group, and a 4-phenylphenyl group.

The number of carbon atoms of the monovalent heterocyclic group as each of R^{A}, R^{B}, R^{C}, R^{D}, and R^{E} is usually 2 to 30 without including the number of carbon atoms of a substituent. Examples of the monovalent heterocyclic group include a group obtained by removing, from a heterocyclic compound such as furan, thiophene, pyrrole, pyrroline, pyrrolidine, oxazole, isoxazole, thiazole, isothiazole, imidazole, imidazoline, imidazolidine, pyrazole, pyrazoline, prazolidine, furazan, triazole, thiadiazole, oxadiazole, tetrazole, pyran, pyridine, piperidine, thiopyran, pyridazine, pyrimidine, pyrazine, piperazine, morpholine, triazine, benzofuran, isobenzofuran, benzothiophene, indole, isoindole, indolizine, indoline, isoindoline, chromene, chroman, isochromane, benzopyran, quinoline, isoquinoline, quinolidine, benzimidazole, benzothiazole, indazole, naphthyridine, quinoxaline, quinazoline, quinazolidine, cinnoline, phthalazine, purine, pteridine, carbazole, xanthene, phenanthridine, acridine, B-carboline, perimidine, phenanthroline, thianthrene, phenoxathiine, phenoxazine, phenothiazine, or phenazine, one hydrogen atom directly bonded to a carbon atom constituting the ring.

In the present specification, examples of the substituent include a halogen atom (a fluorine atom, a chlorine atom, a bromine atom, an iodine atom, or the like), a cyano group, an alkyl group, an alkyloxy group, a haloalkyl group, an aryl group, and a monovalent heterocyclic group.

Each of R^{A}, R^{B}, R^{C}, R^{D}, and R^{E} is preferably an alkyl group having 1 to 30 carbon atoms, a cycloalkyl group having 3 to 30 carbon atoms, or an aryl group having 6 to 30 carbon atoms from a viewpoint of maintaining heat resistance and suppressing aggregation between molecules.

Examples of the divalent polycyclic condensed ring group as X include groups represented by formulas (X-1) to (X-40).

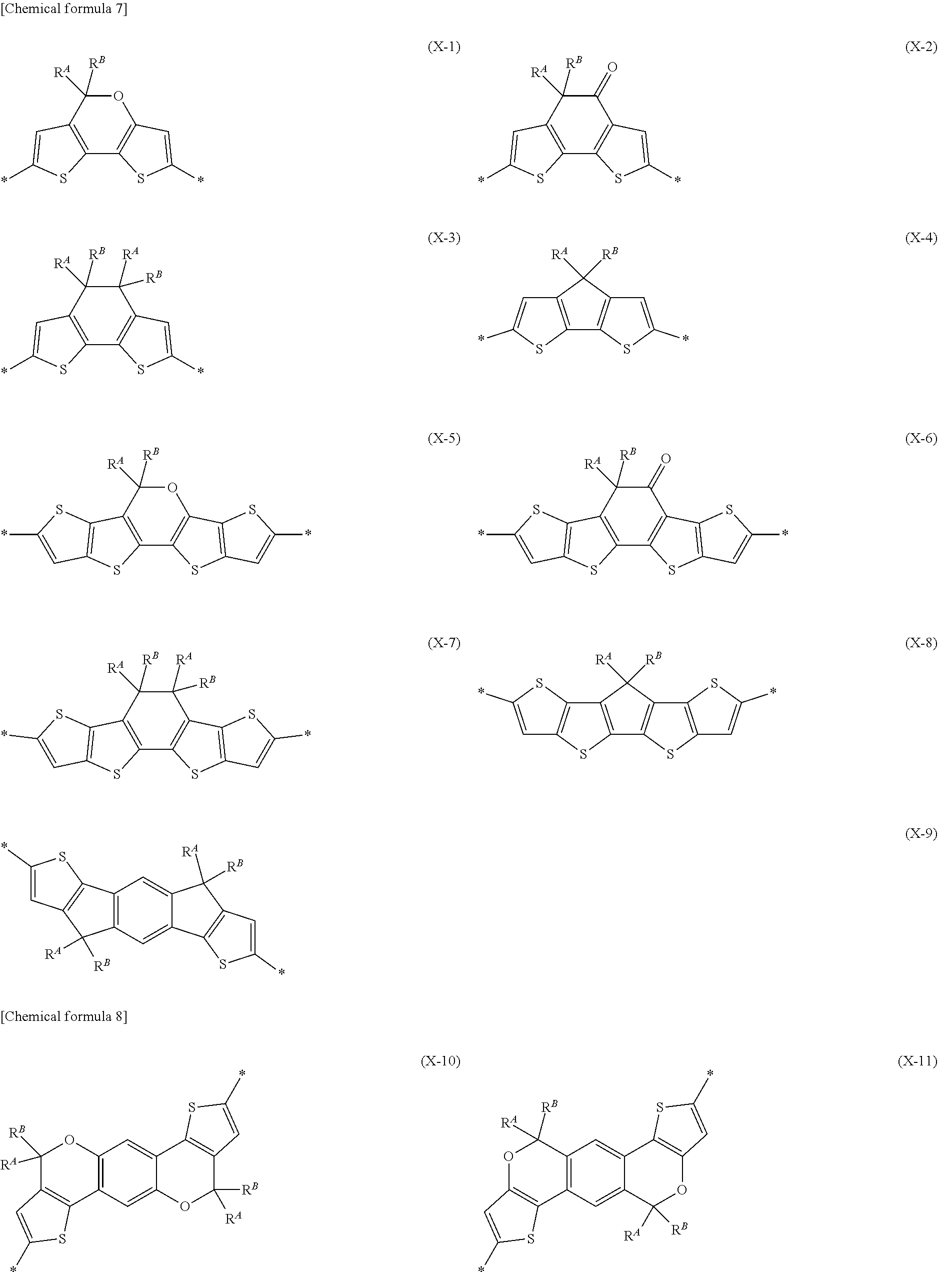

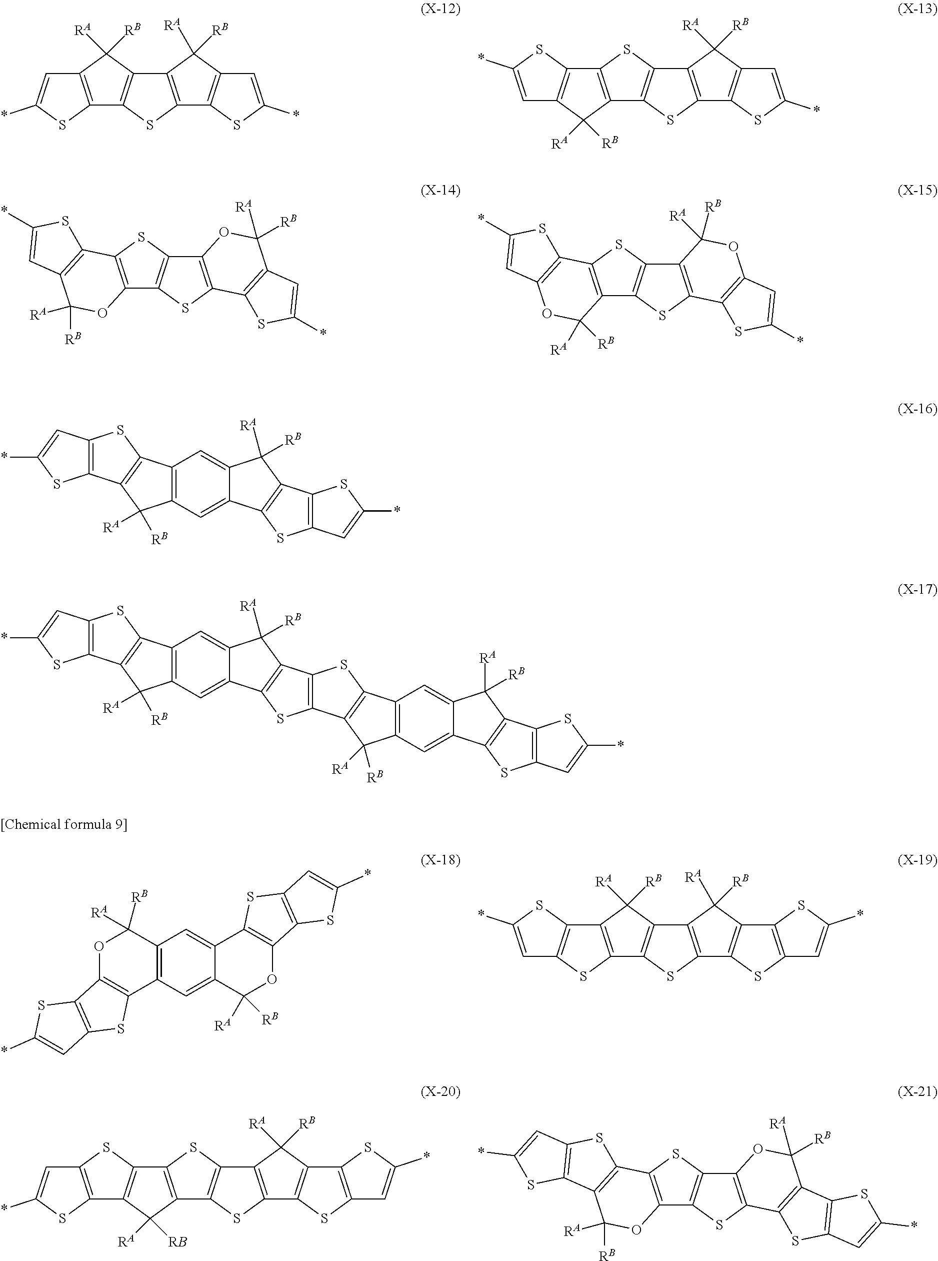

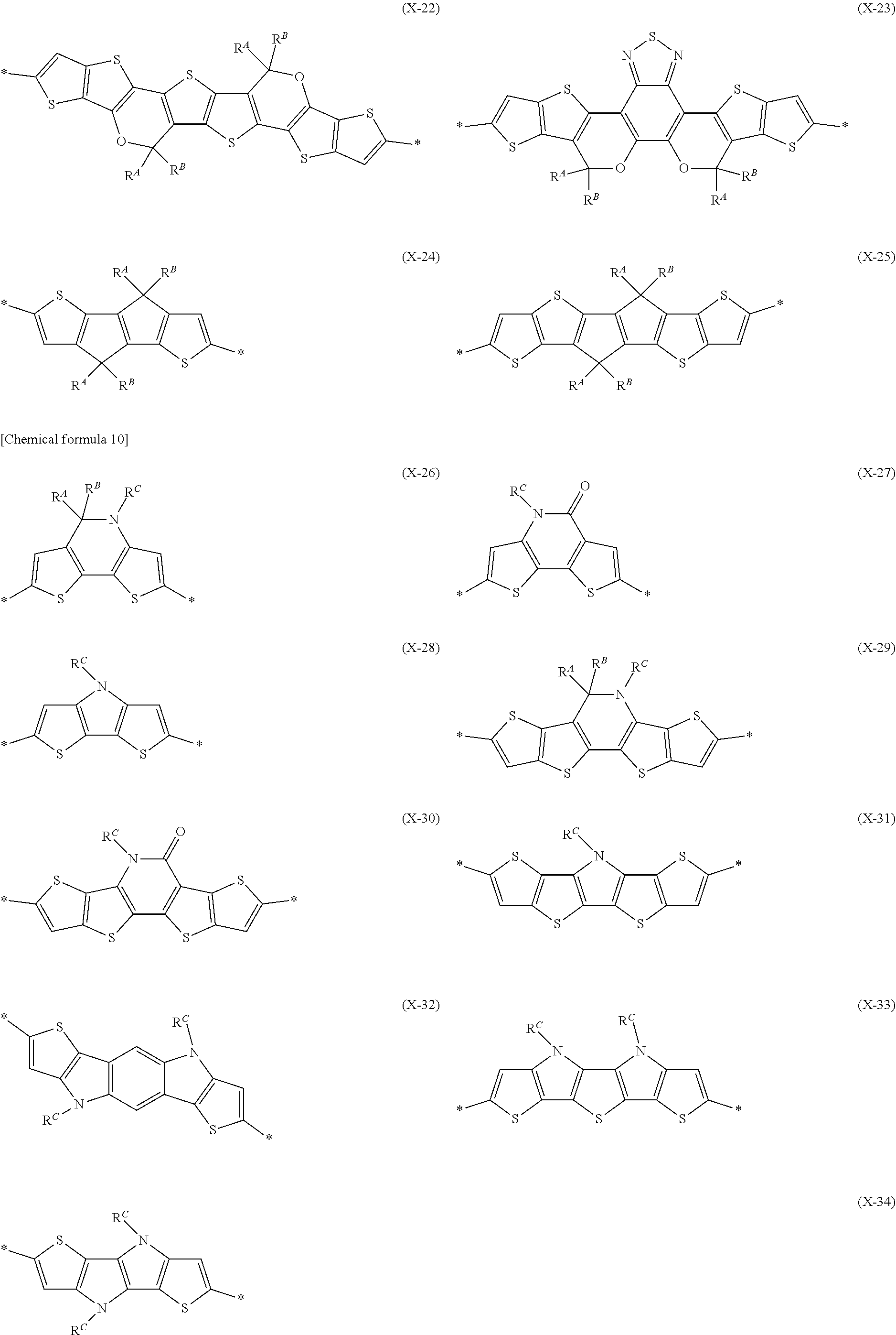

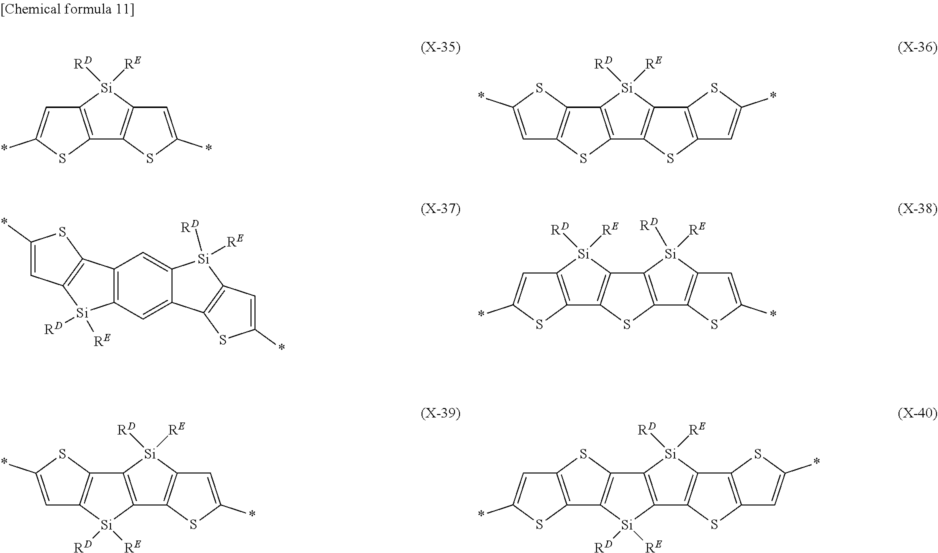

The divalent polycyclic condensed ring group having an sp3 carbon atom as a constituent element as X is preferably a group represented by any one of formulas (X-1), (X-4), (X-5), (X-8) to (X-22), (X-24), and (X-25), and more preferably a group represented by any one of formulas (X-1), (X-4), (X-5), (X-8), (X-9), (X-13), (X-16), (X-24), and (X-25) from a viewpoint of suppressing aggregation between molecules.

The divalent polycyclic condensed ring group having a nitrogen atom as a constituent element as X is preferably a group represented by any one of formulas (X-26) to (X-28) and (X-34) from a viewpoint of suppressing aggregation between molecules.

The divalent polycyclic condensed ring group having a silicon atom as a constituent element as X is preferably a group represented by formula (X-35) or (X-36) from a viewpoint of suppressing aggregation between molecules.

The divalent polycyclic condensed ring group as X is preferably a divalent polycyclic condensed ring group having an sp3 carbon atom as a constituent element or a divalent polycyclic condensed ring group having a nitrogen atom as a constituent element, and more preferably a divalent polycyclic condensed ring group having an sp3 carbon atom as a constituent element because raw materials are easily available and synthesis difficulty is low.

By having such a divalent polycyclic condensed ring group as X, the compound of the present embodiment is a compound having high linearity and flatness, and can be suitably used as an EO compound.

R¹ and R² each independently represent an alkyl group, a haloalkyl group, an acyloxyalkyl group, a silyloxyalkyl group, an aryl group, —R²¹—OH (in which R²¹ represents a divalent hydrocarbon group), —R²²—NH₂ (in which R²² represents a divalent hydrocarbon group), —R²³—SH (in which R²³ represents a divalent hydrocarbon group), or —R²⁴—NCO (in which R²⁴ represents a divalent hydrocarbon group). These groups may each have a crosslinkable group. R¹ and R² may be bonded to each other to form a ring together with atoms to which R¹ and R² are bonded.

Examples of the alkyl group as each of R¹ and R² include the alkyl groups exemplified for R^{A}, R^{B}, R^{C}, R^{D}, and R^{E}. The number of carbon atoms of the alkyl group is preferably 1 to 20, more preferably 1 to 10, and still more preferably 1 to 5.

The haloalkyl group as each of R¹ and R² is an alkyl group having one or more halogen atoms (for example, a fluorine atom, a chlorine atom, a bromine atom, or an iodine atom) as substituents. The number of carbon atoms of the haloalkyl group is preferably 1 to 20, more preferably 1 to 10, and still more preferably 1 to 5. Specific examples of the haloalkyl group include a fluoromethyl group, a difluoromethyl group, a trifluoromethyl group, a 2-fluoroethyl group, a 1,2-difluoroethyl group, a chloromethyl group, a 2-chloroethyl group, a 1,2-dichloroethyl group, a bromomethyl group, a 2-bromoethyl group, a 1-bromopropyl group, a 2-bromopropyl group, a 3-bromopropyl group, and an iodomethyl group.

Examples of the acyloxyalkyl group as each of R¹ and R² include an alkyl group having one or more acyloxy groups as substituents. The number of carbon atoms of the acyloxyalkyl group is preferably 2 to 20, more preferably 3 to 10, and still more preferably 3 to 7.

Examples of the trialkylsilyloxyalkyl group, the aryldialkylsilyloxyalkyl group, and the alkyldiarylsilyloxyalkyl group as each of R¹ and R² include an alkyl group having one or more trialkylsilyloxy groups as substituents, an alkyl group having one or more aryldialkylsilyloxy groups as substituents, and an alkyl group having one or more alkyldiarylsilyloxy groups as substituents. The number of carbon atoms of each of the trialkylsilyloxyalkyl group, the aryldialkylsilyloxyalkyl group, and the alkyldiarylsilyloxyalkyl group is preferably 5 to 25, more preferably 10 to 22, and still more preferably 12 to 20.

Examples of the aryl group as each of R¹ and R² include the aryl groups exemplified for R^{A}, R^{B}, R^{C}, R^{D}, and R^{E}. The number of carbon atoms of the aryl group is preferably 6 to 20, and more preferably 6 to 10.

Examples of the divalent hydrocarbon group as each of R²¹, R²², R²³, and R²⁴ in each of R¹ and R² include an alkanediyl group and a cycloalkanediyl group. Specific examples of the alkanediyl group include: a linear alkanediyl group such as a methylene group, an ethylene group, a trimethylene group, a tetramethylene group, a pentamethylene group, a hexamethylene group, an octamethylene group, a decamethylene group, or a dodecamethylene group; and a branched alkanediyl group such as a propylene group, an isopropylene group, an isobutylene group, a 2-methyltrimethylene group, an isopentylene group, an isohexylene group, an isooctylene group, a 2-ethylhexylene group, or an isodecylene group. Specific examples of the cycloalkanediyl group include a cyclopropylene group, a cyclopentylene group, a cyclohexylene group, and a cyclododecylene group. The number of carbon atoms of the alkanediyl group is preferably 1 to 20, more preferably 1 to 10, and still more preferably 1 to 5. The number of carbon atoms of the cycloalkanediyl group is preferably 3 to 20.

Each of R¹ and R² is preferably an alkyl group having 1 to 10 carbon atoms, an acyloxyalkyl group having 3 to 10 carbon atoms, a silyloxyalkyl group having 5 to 10 carbon atoms, an aryl group having 6 to 10 carbon atoms, —R²¹—OH (R²¹ represents an alkanediyl group having 1 to 10 carbon atoms), —R²²—NH₂ (R²² represents an alkanediyl group having 1 to 10 carbon atoms), —R²³—SH (R²³ represents an alkanediyl group having 1 to 10 carbon atoms), or —R²⁴—NCO (in which R²⁴ represents an alkanediyl group having 1 to 10 carbon atoms), and more preferably an alkyl group having 1 to 5 carbon atoms, an acyloxyalkyl group having 3 to 7 carbon atoms, a silyloxyalkyl group having 6 to 9 carbon atoms, —R²¹—OH (R²¹ represents an alkanediyl group having 1 to 5 carbon atoms), —R²²—NH₂ (R²² represents an alkanediyl group having 1 to 5 carbon atoms), —R²³—SH (R²³ represents an alkanediyl group having 1 to 5 carbon atoms), or —R²⁴—NCO (in which R²⁴ represents an alkanediyl group having 1 to 5 carbon atoms) from a viewpoint of exhibiting excellent EO characteristics.

Each of R¹ and R² may have a crosslinkable group. The crosslinkable group means a group that reacts with an identical or different group of another molecule located in the vicinity by irradiation with heat and/or active energy rays to generate a novel chemical bond. Examples of the crosslinking group include: a radically polymerizable group such as a (meth)acryloyloxy group or a styryl group (vinylphenyl group); and a Diels-Alder polymerizable group that reacts with a dienophile, such as an anthracenyl group or a benzocyclobutenyl group.

R¹ and R² may be bonded to each other to form a ring together with atoms to which R¹ and R² are bonded. When R¹ and R² are bonded to each other to form a ring, the ring is preferably a 5-membered ring or a 6-membered ring from a viewpoint of ensuring stability. The ring is preferably an aliphatic ring.

R³ represents an alkyl group, an alkyloxy group, an aryl group, an aryloxy group, an aralkyloxy group, a silyloxyalkyl group, an alkenyloxy group, an alkynyloxy group, a hydroxy group, an amino group, a sulfanyl group, an isocyanate group, —R³¹—OH (R³¹ represents a divalent hydrocarbon group), —O—R³²—OH (R³² represents a divalent hydrocarbon group), —R³³—NH₂ (R³³ represents a divalent hydrocarbon group), —R³⁴—SH (R³⁴ represents a divalent hydrocarbon group), —R³⁵—NCO (R³⁵ represents a divalent hydrocarbon group), or —OC(═O)R⁴¹ (R⁴¹ represents a monovalent hydrocarbon group). These groups may each have a crosslinkable group. When there is a plurality of R³s, R³s may be the same as or different from each other. R³ may be bonded to R¹ or R² to form a ring together with atoms to which R³ and R¹ or R² are bonded.

Examples of the alkyl group and the alkyloxy group as R³ include the alkyl groups and the alkyloxy groups exemplified for R^{A}, R^{B}, R^{C}, R^{D}, and R^{E}. The number of carbon atoms of the alkyl group is preferably 1 to 20, more preferably 1 to 10, and still more preferably 1 to 5.

Examples of the aryl group and the aryl group of the aryloxy group as R³ include the aryl groups exemplified for R^{A}, R^{B}, R^{C}, R^{D}, and R^{E}. The number of carbon atoms of the aryl group is preferably 6 to 20, and more preferably 6 to 10.

Examples of the aralkyl group of the aralkyloxy group as R³ include an alkyl group having one or more aralkyl groups as substituents. Specific examples of the aralkyl group include a benzyl group, a 1-phenylethyl group, a phenethyl group, a 1-naphthylmethyl group, a 2-naphthylmethyl group, a 1-naphthylethyl group, and a 2-naphthylethyl group.

Examples of the trialkylsilyloxyalkyl group, the aryldialkylsilyloxyalkyl group, and the alkyldiarylsilyloxyalkyl group as R³ include the trialkylsilyloxyalkyl groups, the aryldialkylsilyloxyalkyl groups, and the alkyldiarylsilyloxyalkyl groups exemplified for R¹ and R².

Examples of the alkenyl group of the alkenyloxy group as R³ include an alkenyl group having 2 to 20 carbon atoms. Specific examples of the alkenyl group include an ethenyl group, a 1-propenyl group, a 2-propenyl group, a 1-methylethenyl group, a 1-butenyl group, a 2-butenyl group, a 3-butenyl group, a 1-methyl-1-propenyl group, a 1-methyl-2-propenyl group, a 2-methyl-1-propenyl group, and a 2-methyl-2-propenyl group.

Examples of the alkynyl group of the alkynyloxy group as R³ include an alkynyl group having 3 to 20 carbon atoms. Specific examples of the alkynyl group include a 2-propynyl group, a 1-methyl-2-propynyl group, a 1,1-dimethyl-2-propynyl group, a 2-butynyl group, a 3-butynyl group, a 1-pentynyl group, a 2-pentynyl group, a 3-pentynyl group, and a 4-pentynyl group.

In R³, examples of the divalent hydrocarbon group as each of R³¹, R³², R³³, R³⁴, and R³⁵ include the divalent hydrocarbon groups exemplified for R²¹, R²², R²³, and R²⁴.

In R³, examples of the monovalent hydrocarbon group as R⁴¹ include the alkyl groups and the cycloalkyl groups exemplified for R^{A}, R^{B}, R^{C}, R^{D}, and R^{E}.

k represents an integer of 0 to 4. k is preferably 0 or 1, and more preferably 0.

R³ may have a crosslinkable group. Examples of the crosslinkable group include the crosslinkable groups exemplified for R¹ and R².

R³ may be bonded to R¹ or R² to form a ring together with atoms to which R³ and R¹ or R² are bonded. When R³ is bonded to R¹ or R² to form a ring, the ring is preferably a 5-membered ring or a 6-membered ring from a viewpoint of ensuring stability. The ring is preferably an aliphatic ring.

A is a group having an acceptor structure, and represents any one of groups represented by the following formulas (a1), (a2), and (b1).

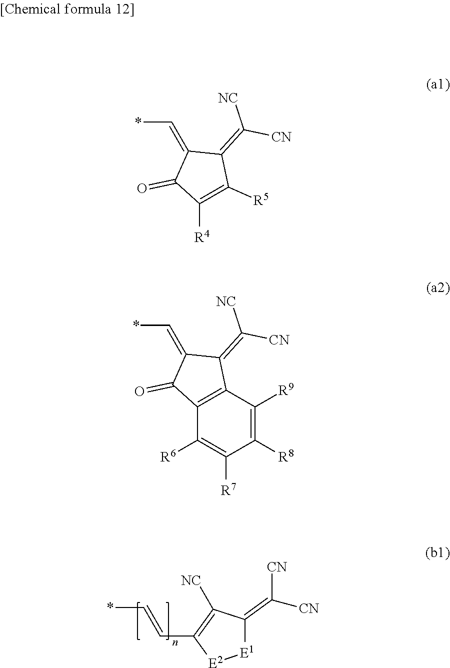

R⁴, R⁵, R⁶, R⁷, R⁸, and R⁹ each independently represent a hydrogen atom, a halogen atom, an alkyl group, a haloalkyl group, a cyano group, an aryl group, or a haloaryl group.

Examples of the halogen atom include a fluorine atom, a chlorine atom, a bromine atom, and an iodine atom. The halogen atom is preferably a fluorine atom.

Examples of the alkyl group, the haloalkyl group, and the aryl group as each of R⁴, R⁵, R⁶, R⁷, R⁸, and R⁹ include the alkyl groups, the haloalkyl groups, and the aryl groups exemplified for R¹ and R². The alkyl group is preferably a methyl group. The haloalkyl group is preferably a trifluoromethyl group. The alkyl group is preferably an aryl group.

The haloaryl group as each of R⁴, R⁵, R⁶, R⁷, R⁸, and R⁹ is an aryl group having one or more halogen atoms (for example, a fluorine atom, a chlorine atom, a bromine atom, or an iodine atom, preferably a fluorine atom) as substituents. The haloaryl group is preferably a pentafluorophenyl group.

E¹ and E² each independently represent —C(R¹⁰)(R¹¹)—, —C(O)—, —O—, or —NR¹²—. Note that at least one of E¹ and E² is —O— or —NR¹²—.

R¹⁰ and R¹¹ each independently represent a hydrogen atom, an alkyl group, a haloalkyl group, an aryl group, or a haloaryl group. Examples of the alkyl group, the haloalkyl group, the aryl group, and the haloaryl group as each of R¹⁰ and R¹¹ include the alkyl groups, the haloalkyl groups, the aryl groups, and the haloaryl groups exemplified for R⁴, R⁵, R⁶, R⁷, R⁸, and R⁹.

R¹² represents a hydrogen atom or an alkyl group. Examples of the alkyl group include the alkyl groups exemplified for R¹ and R².

When A is a group represented by formula (a1) or (a2), m is 0.

When A is a group represented by formula (b1), m and n each independently represent 0 or 1. Note that m+n=1. By satisfying such conditions, the compound represented by formula (1) can suppress a multimerization reaction (for example, a Diels-Alder reaction) between the molecules by heating, and has excellent heat resistance. m is preferably 0, and n is preferably 1. When m is 0 and n is 1, the multimerization reaction between the molecules by heating can be further suppressed.

The compound represented by formula (1) is preferably a compound represented by formula (2a1), a compound represented by formula (2a2), or a compound represented by formula (2b1), more preferably a compound represented by formula (2a2) or a compound represented by formula (2b1), and still more preferably a compound represented by formula (2b1).

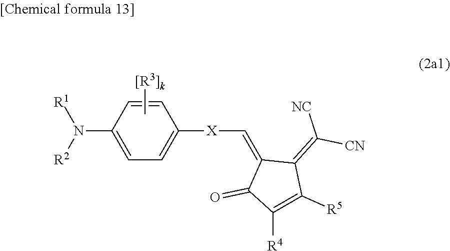

In formula (2a1), X, R¹, R², R³, R⁴, R⁵, and k have the same meanings as those described above.

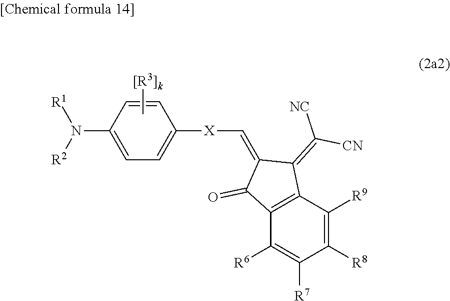

In formula (2a2), X, R¹, R², R³, R⁶, R⁷, R⁸, R⁹, and k have the same meanings as those described above.

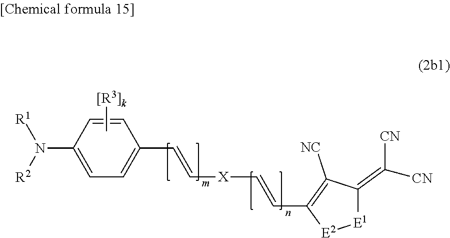

In formula (2b1), X, R¹, R², R³, E¹, E², k, m, and n have the same meanings as those described above.

The compound represented by formula (2b1) is preferably a compound represented by formula (2b1-1) or a compound represented by formula (2b1-2), and more preferably a compound represented by formula (2b1-1).

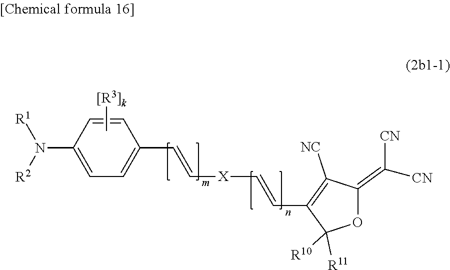

In formula (2b1-1), X, R¹, R², R³, k, m, and n have the same meanings as those described above.

R¹⁰ and R¹¹ each independently represent a methyl group, a trifluoromethyl group, a phenyl group, or a pentafluorophenyl group.

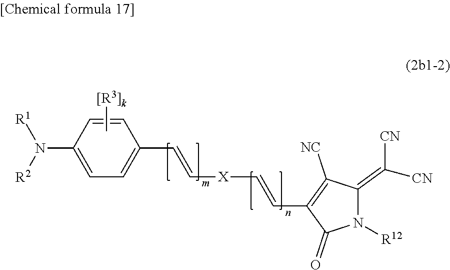

In formula (2b1-2), X, R¹, R², R³, R¹², k, m, and n have the same meanings as those described above.

The compound represented by formula (2b1) (or the compound represented by formula (2b1-1)) is preferably a compound represented by formula (2b1-1a).

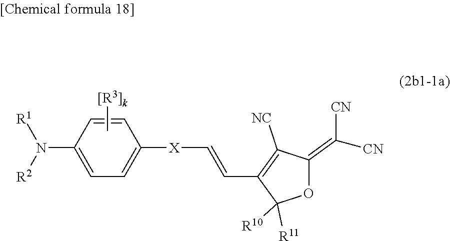

In formula (2b1-1a), X, R¹, R², R³, R¹⁰, R¹¹, and k have the same meanings as those described above.

Examples of the compound represented by formula (1) include compounds represented by formulas (1)-1 to (1)-210.

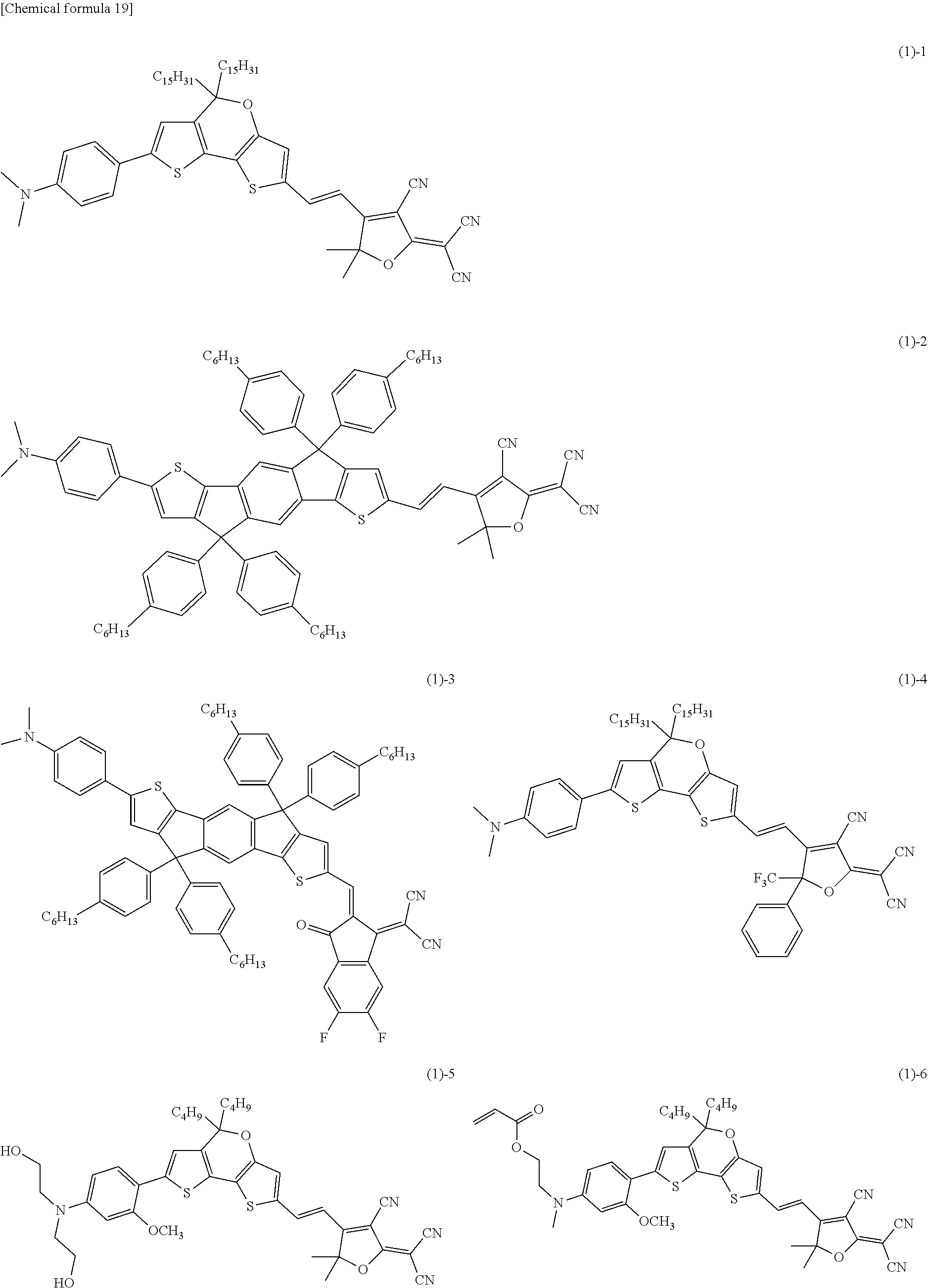

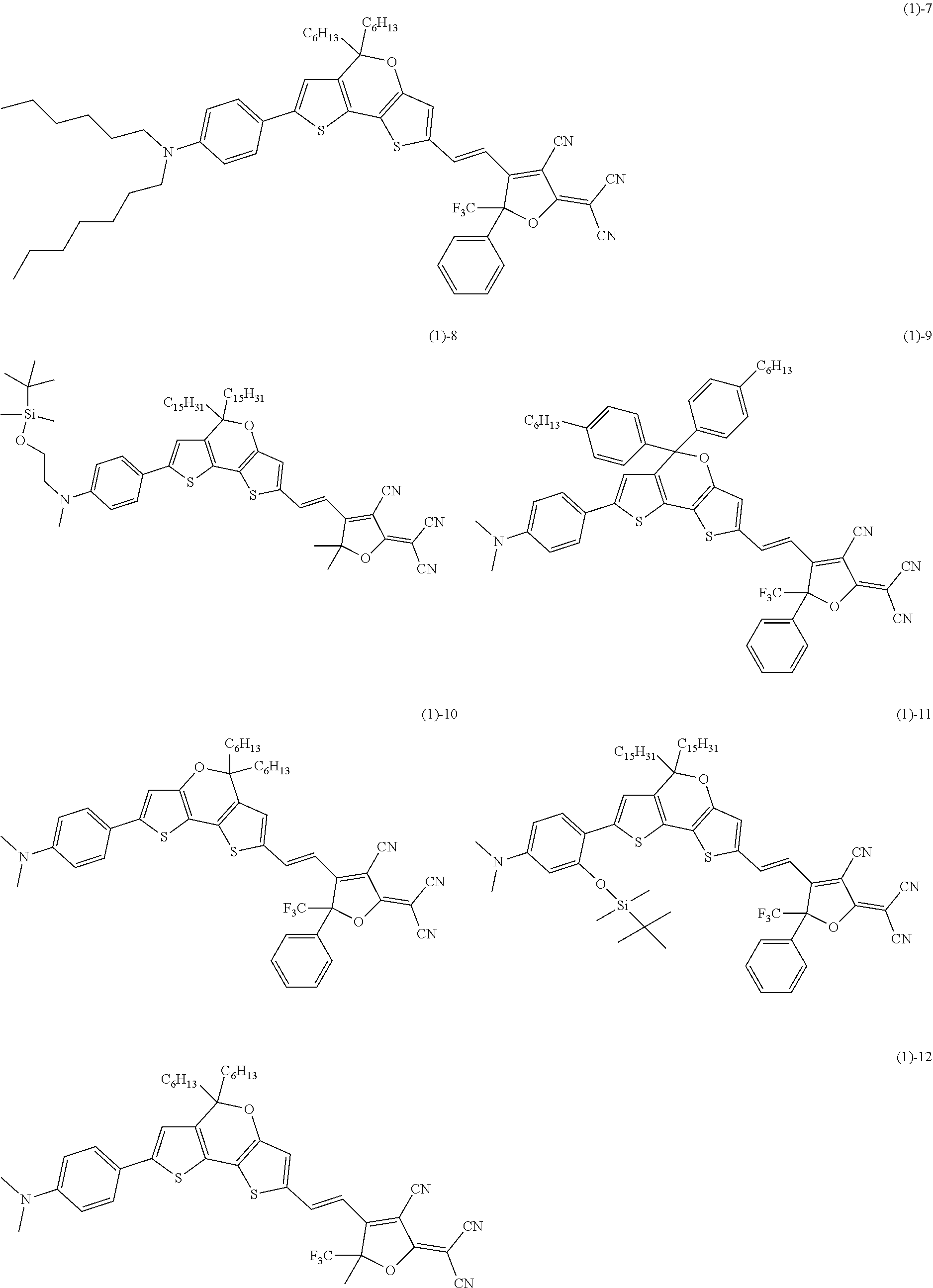

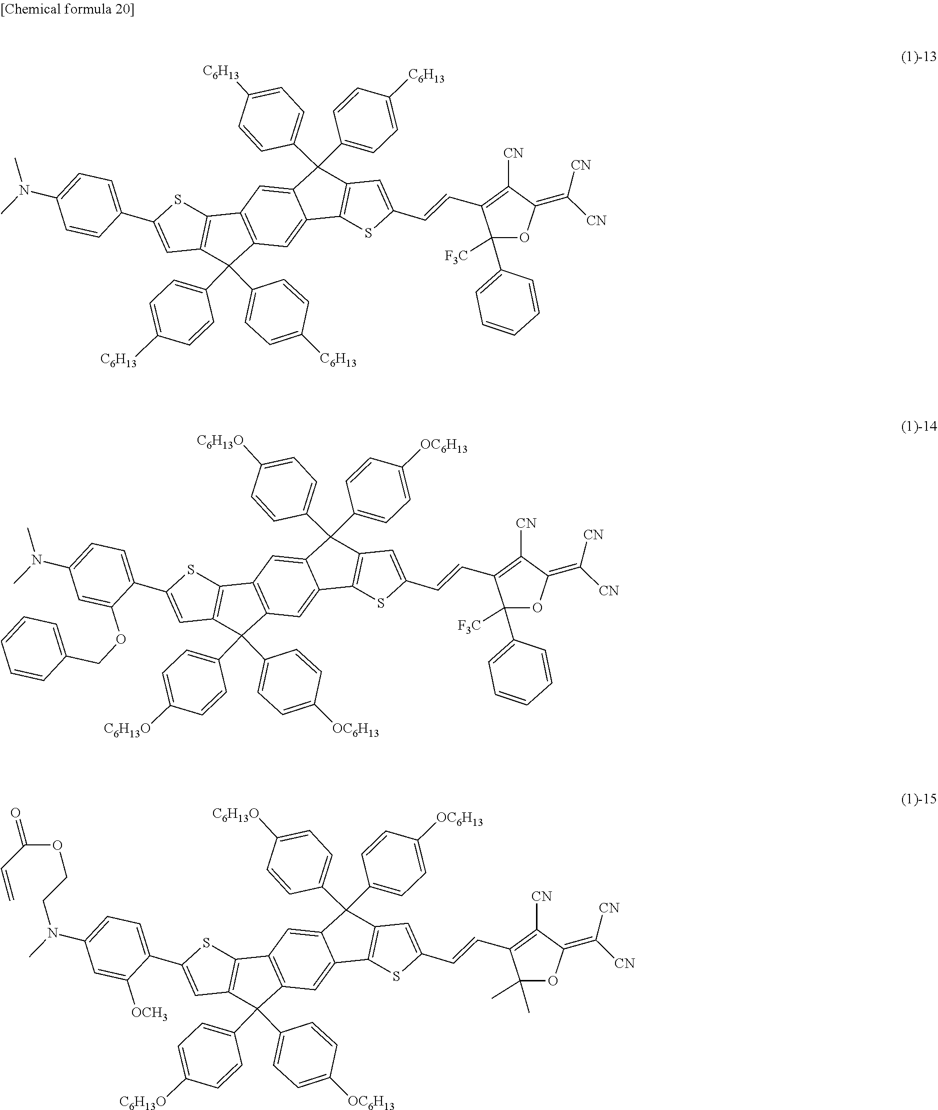

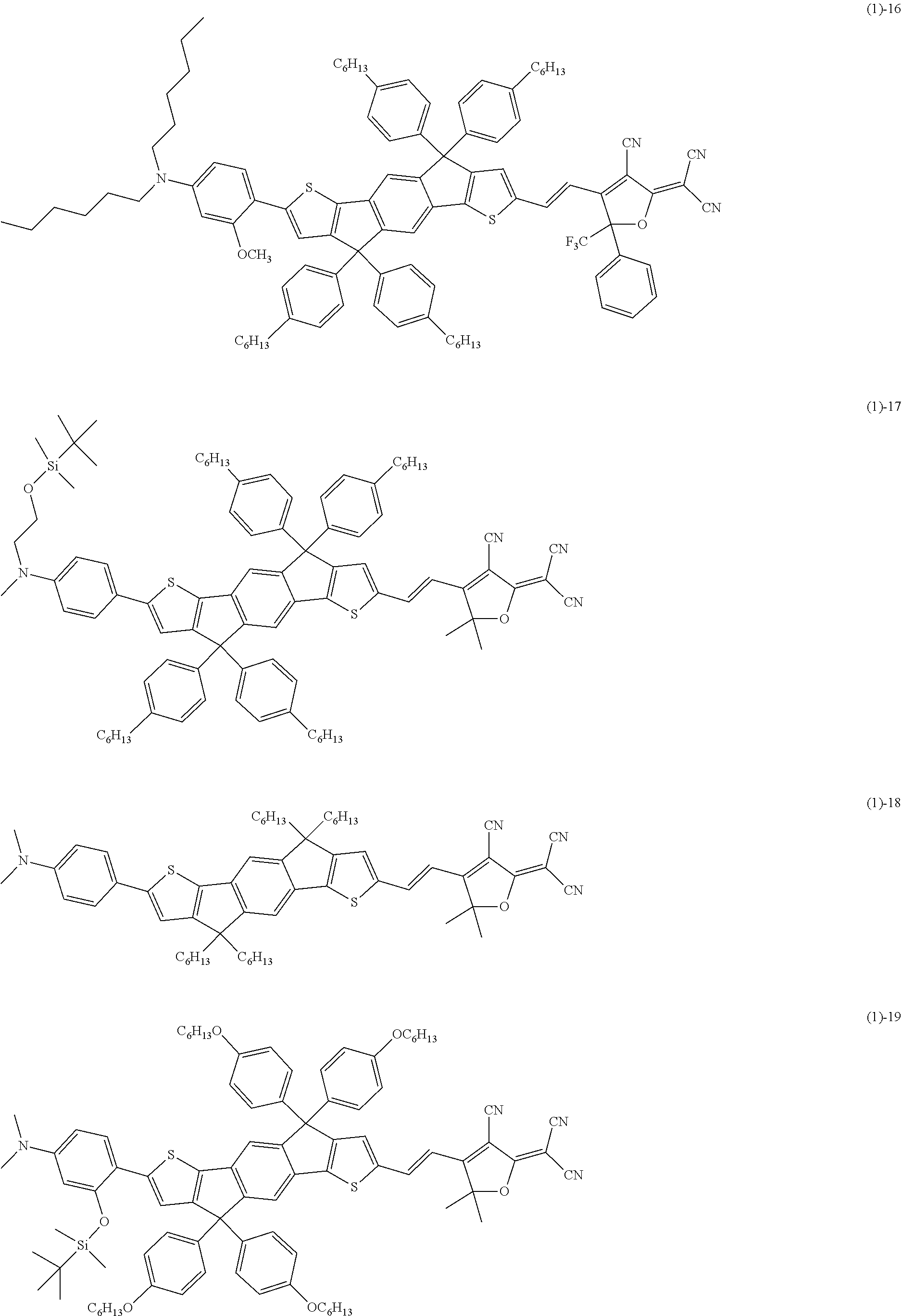

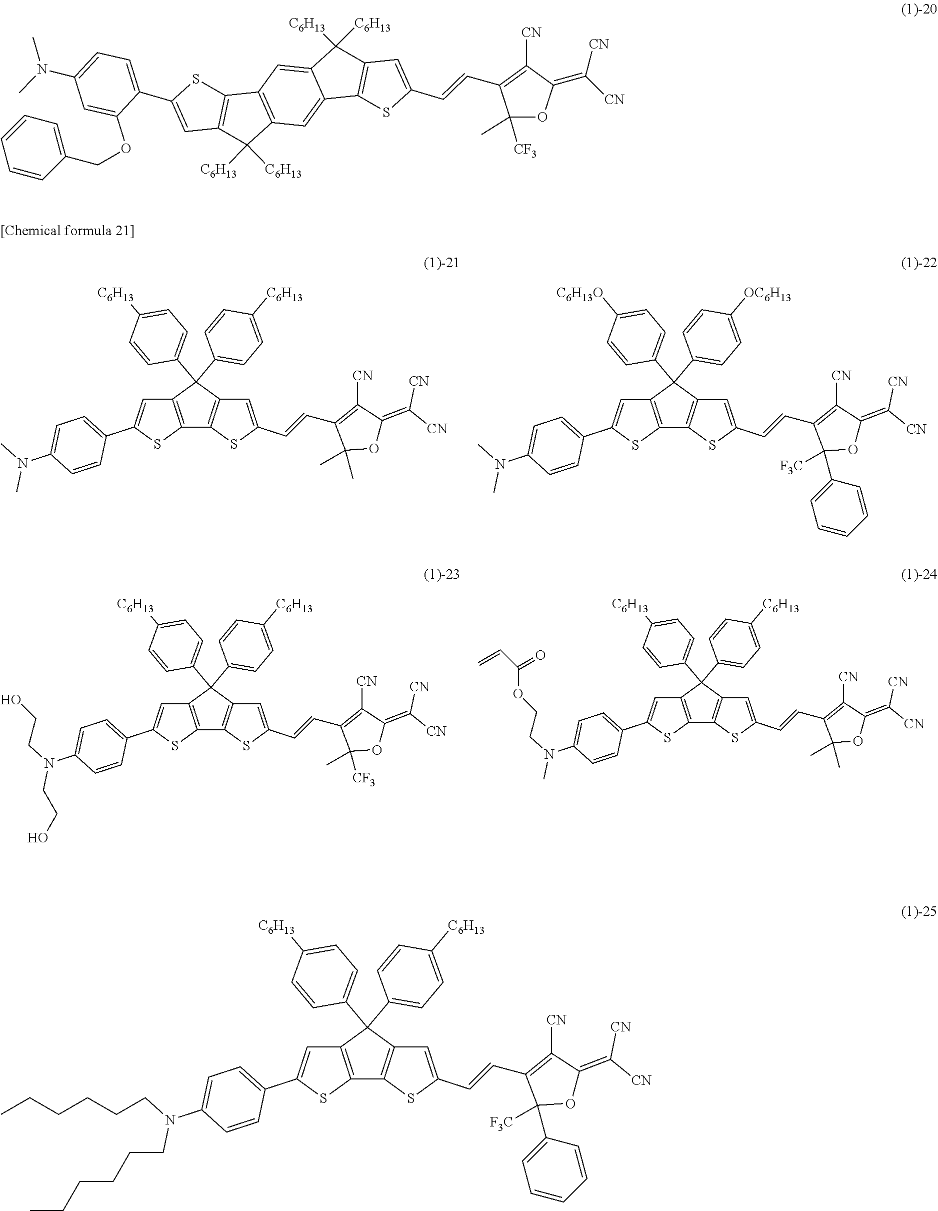

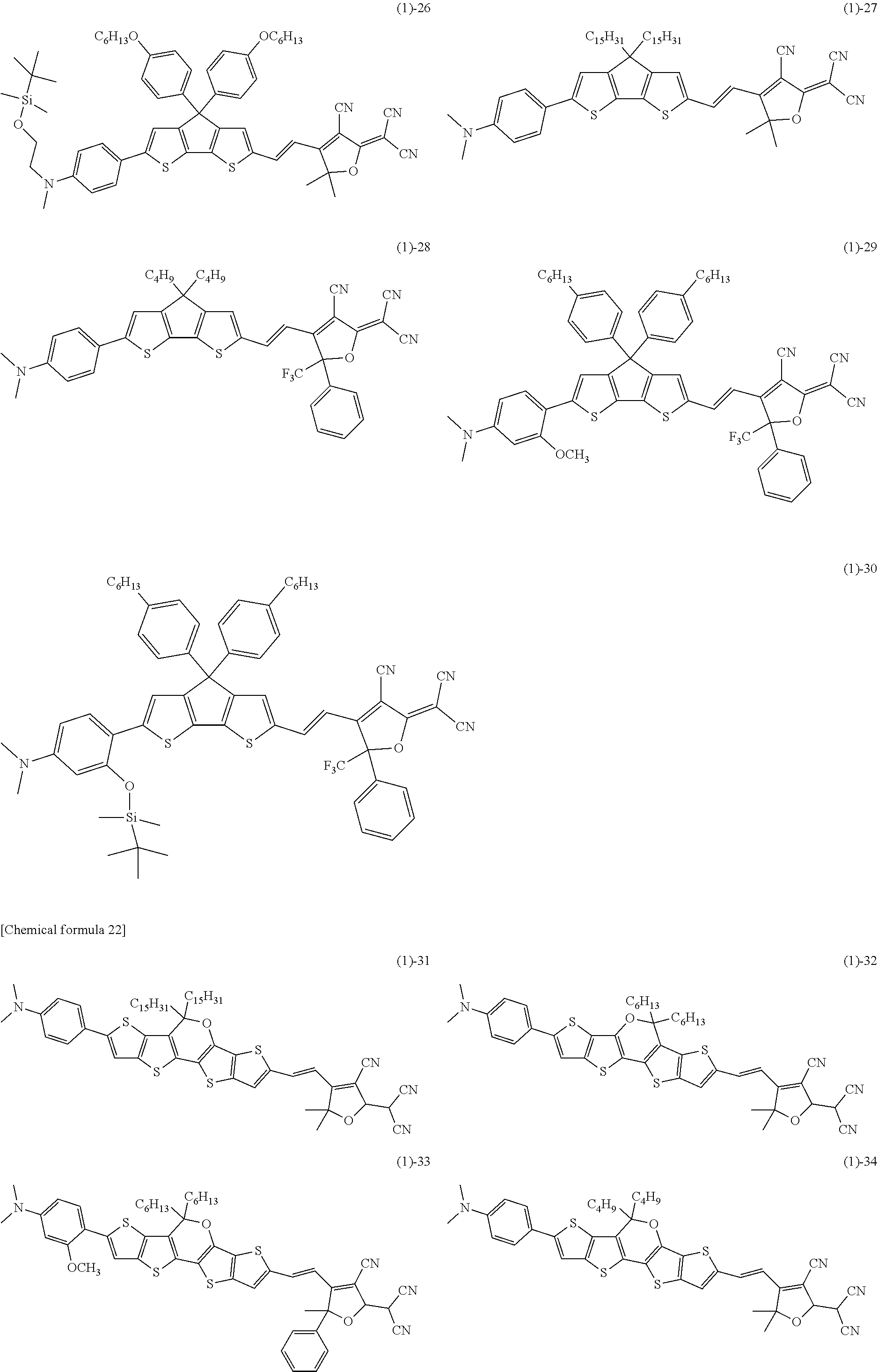

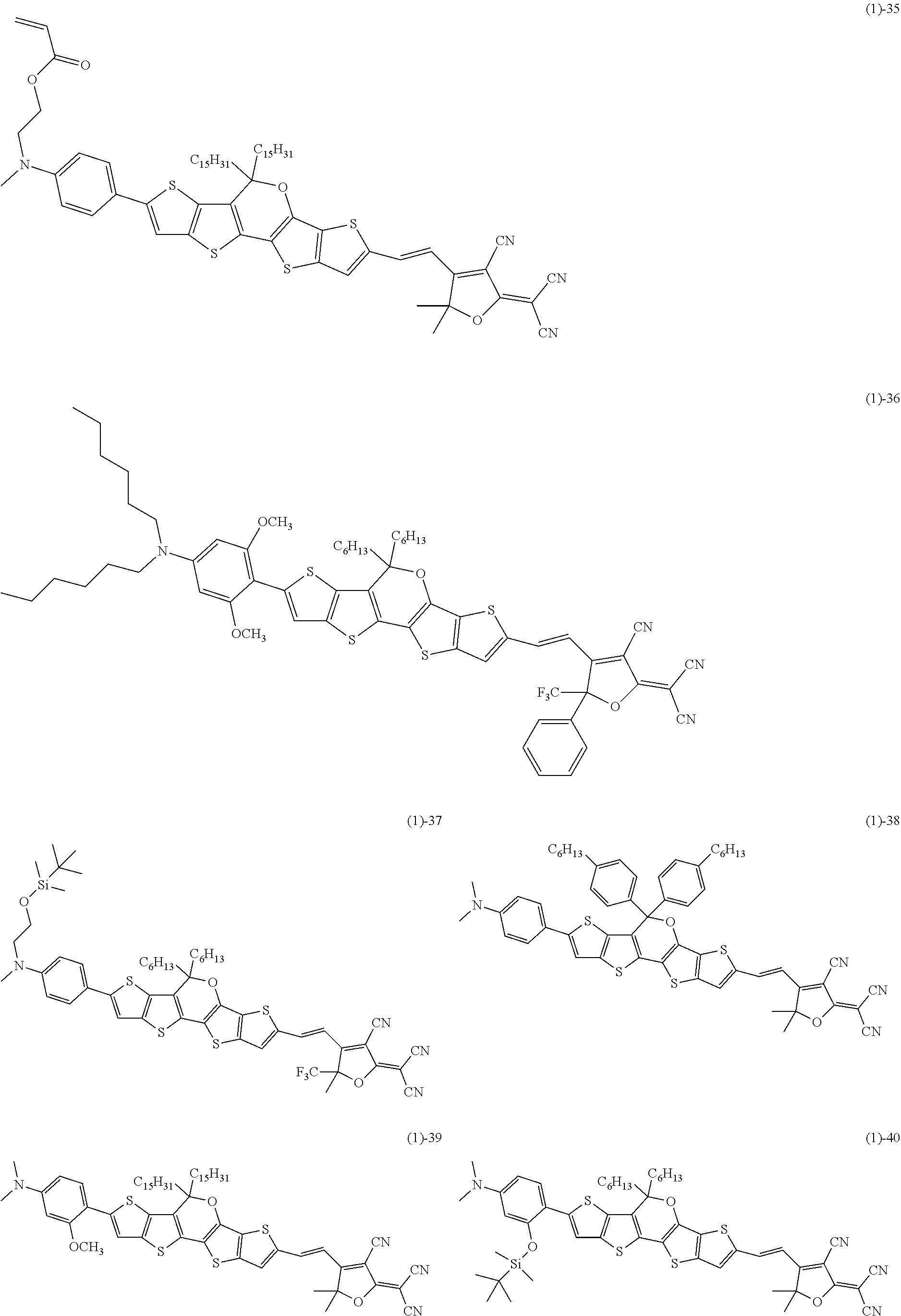

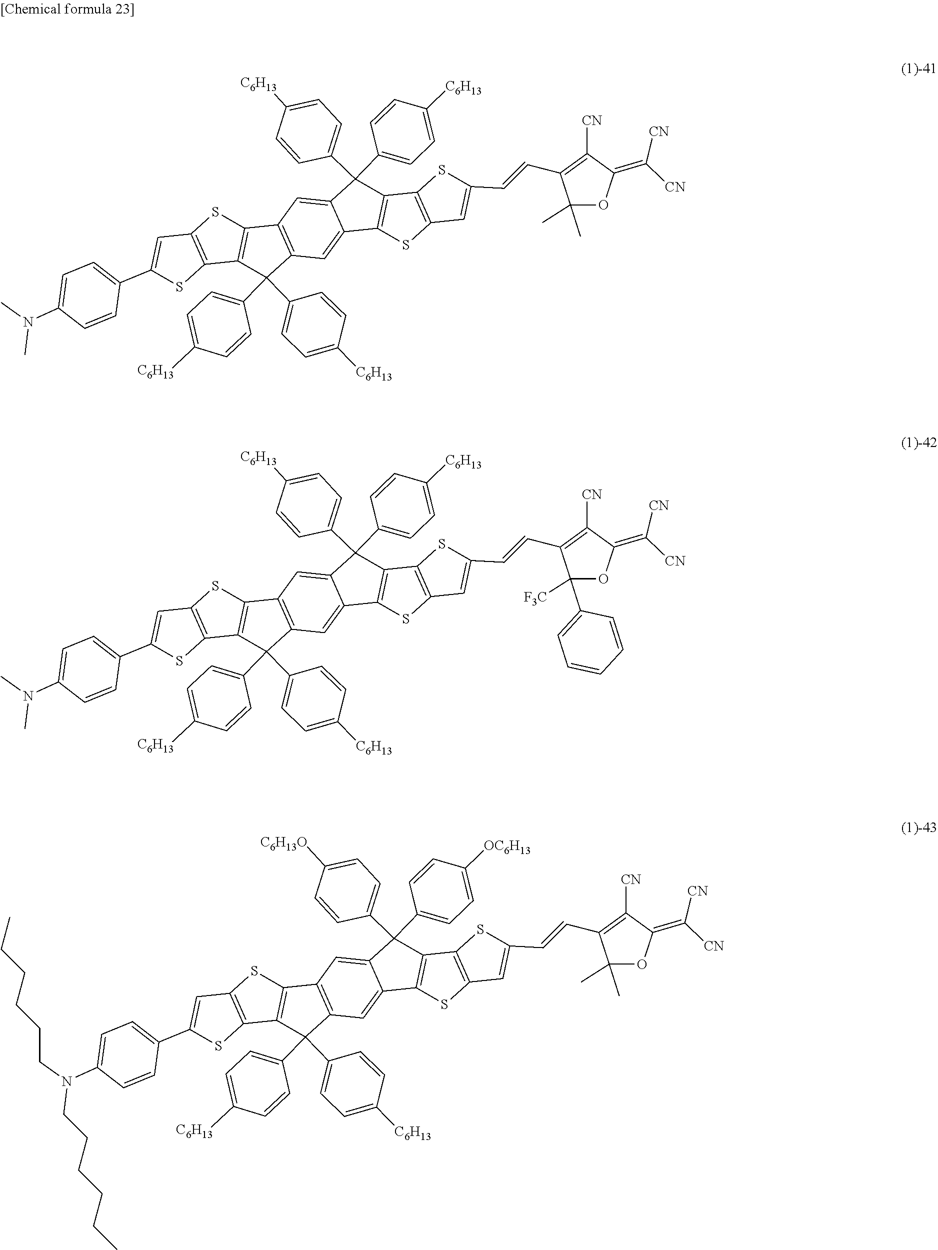

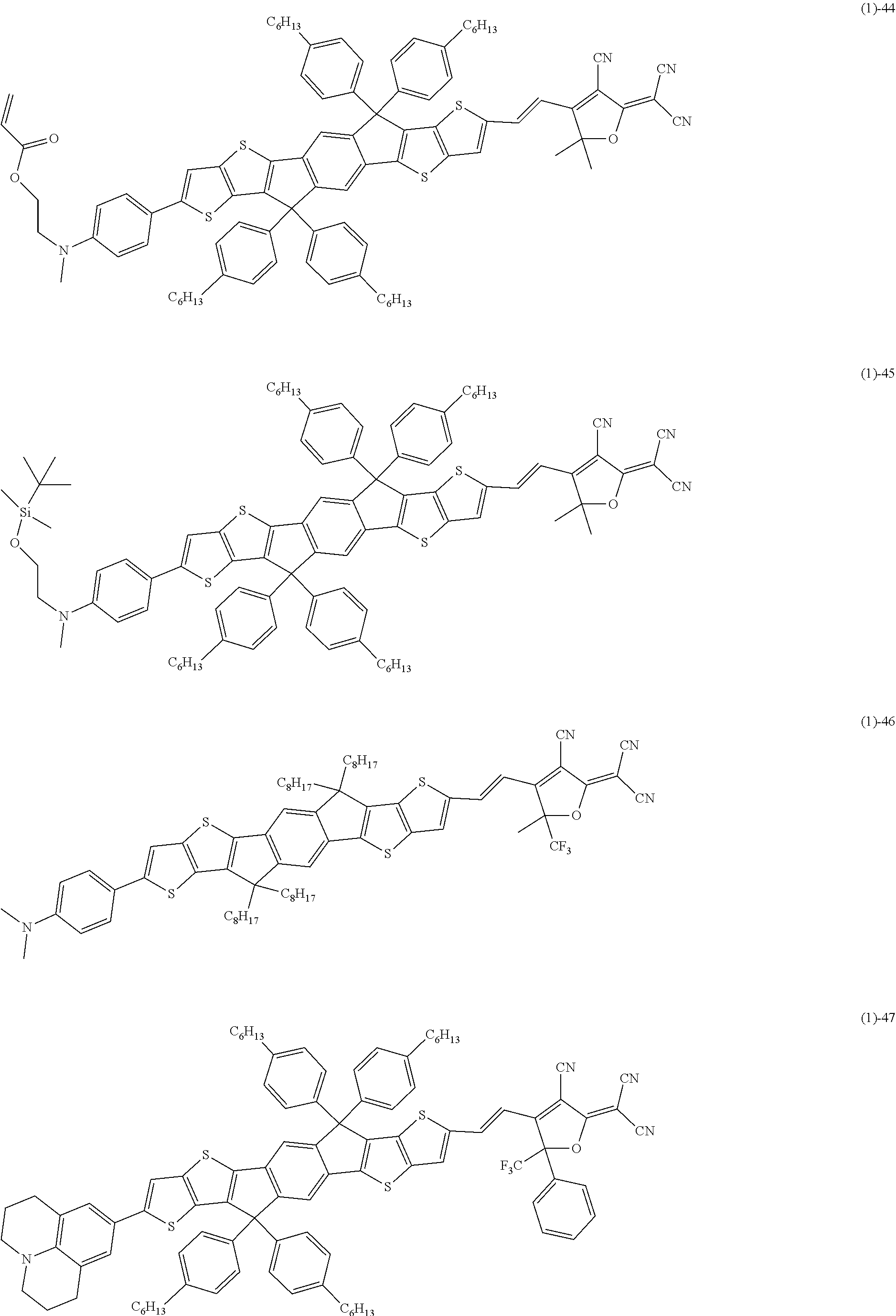

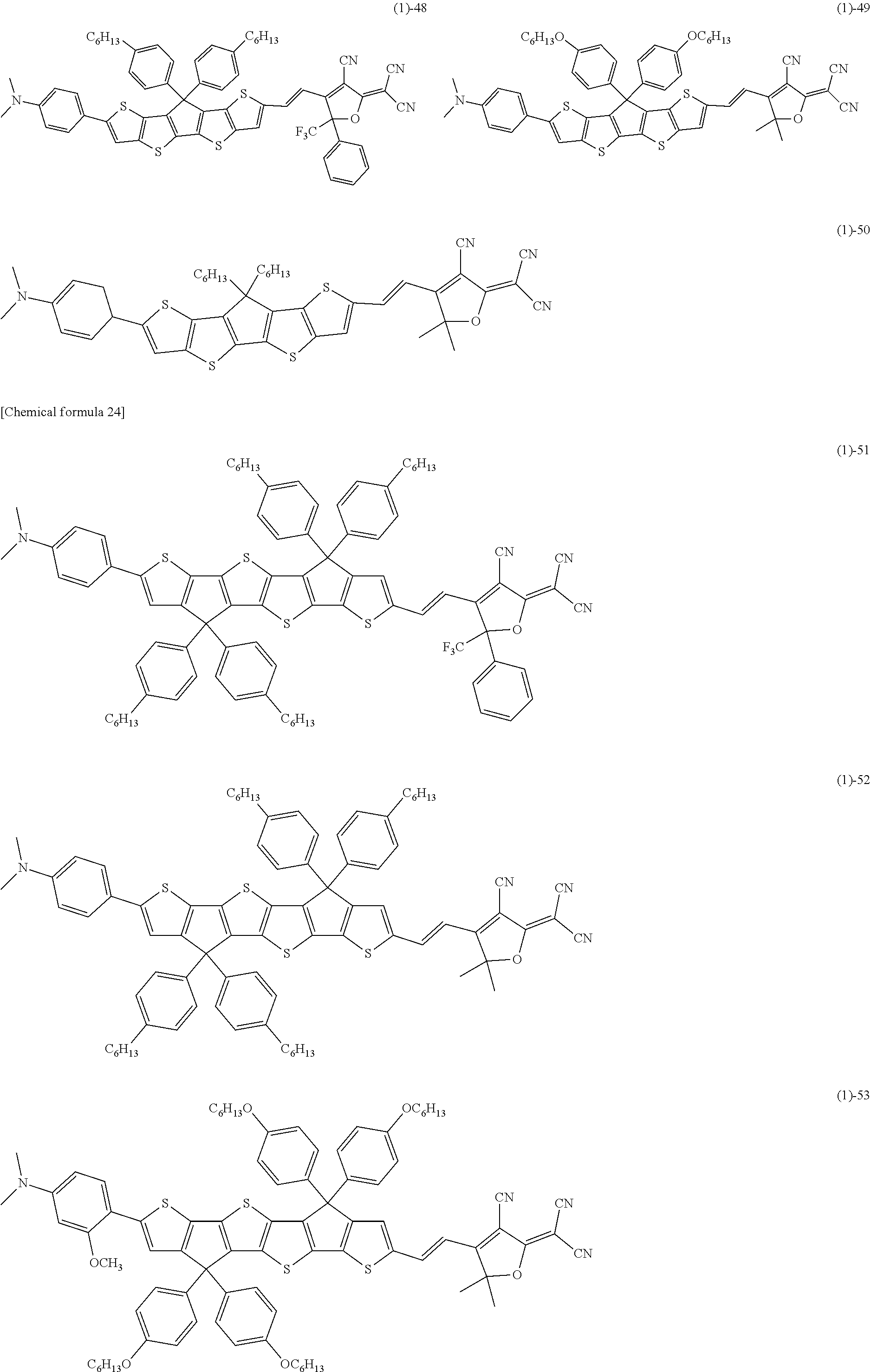

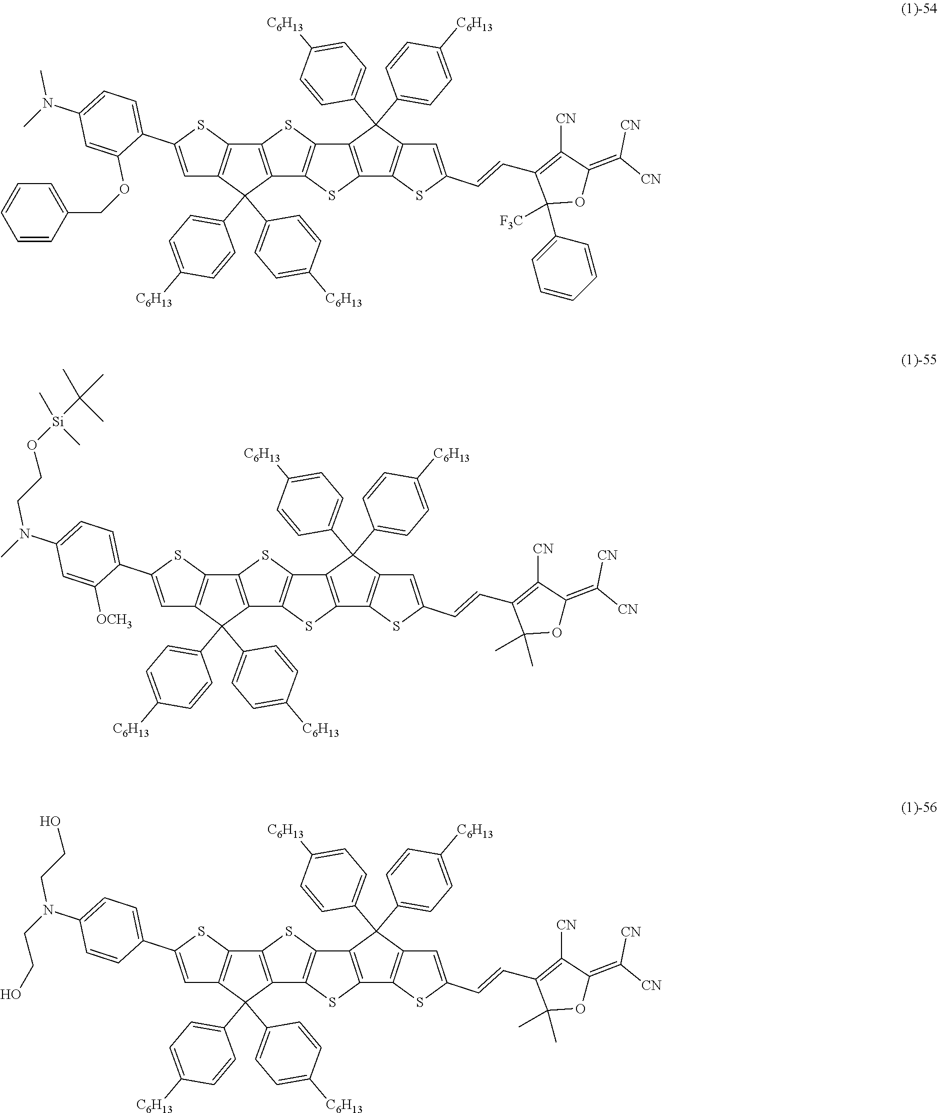

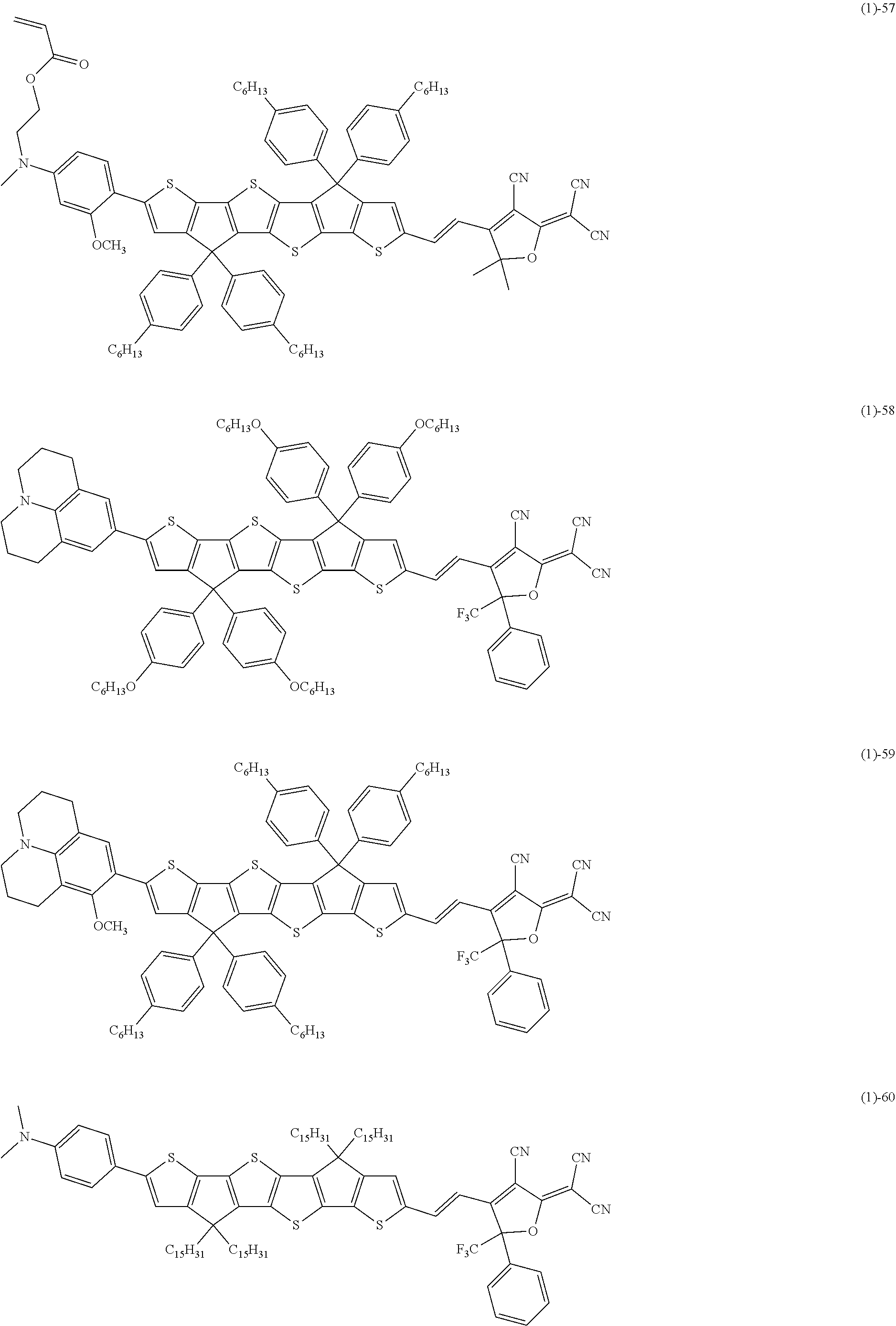

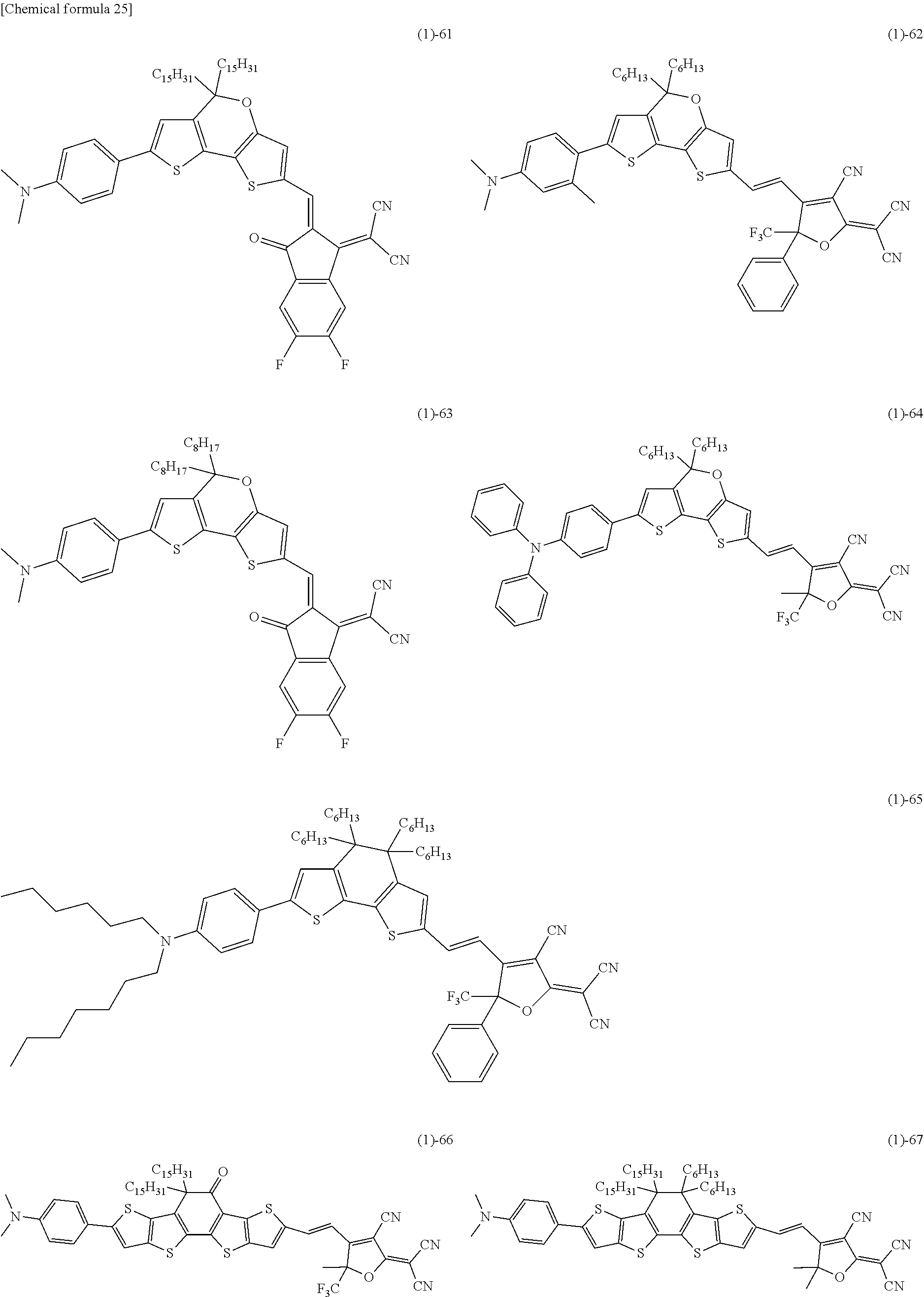

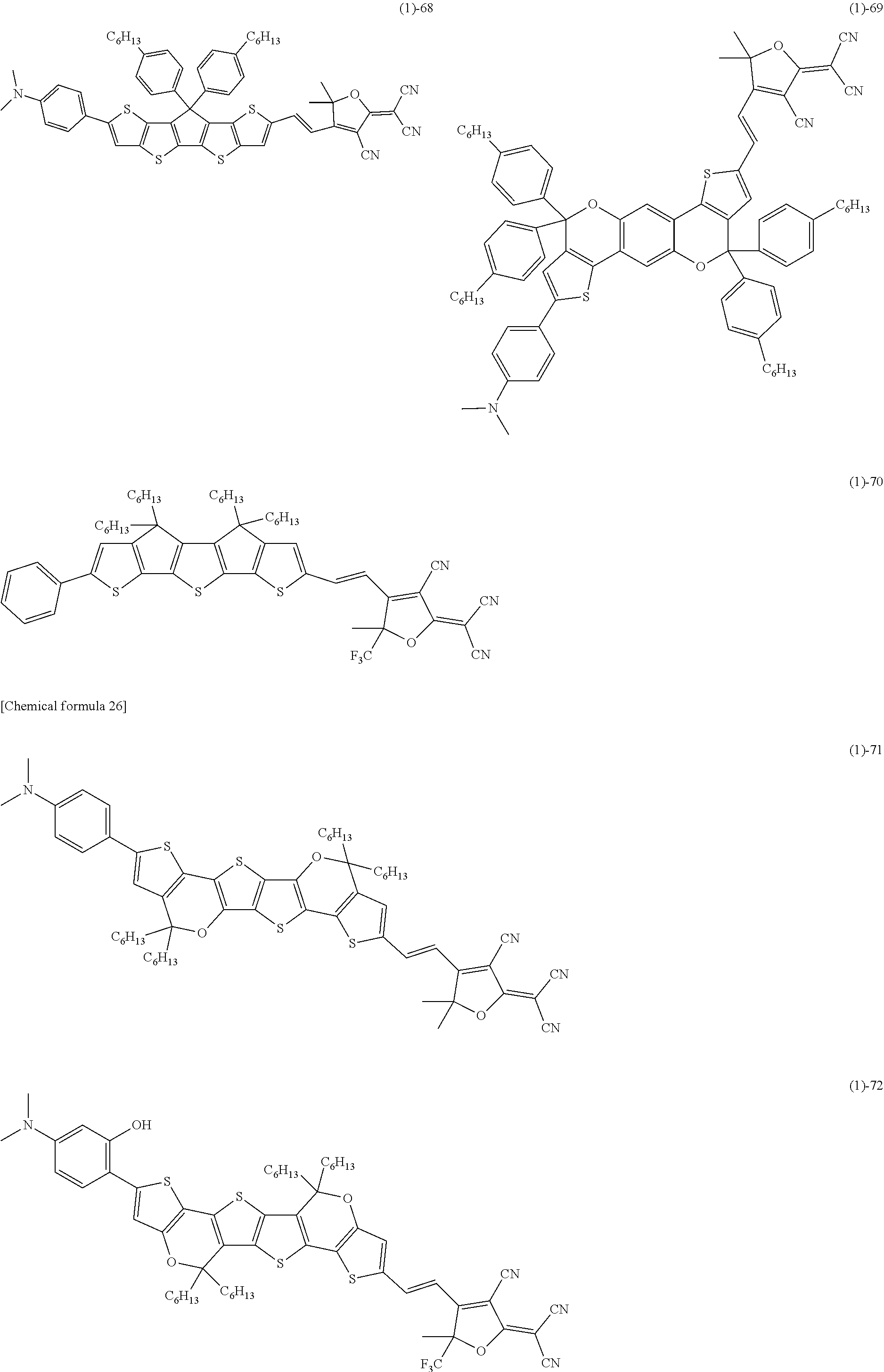

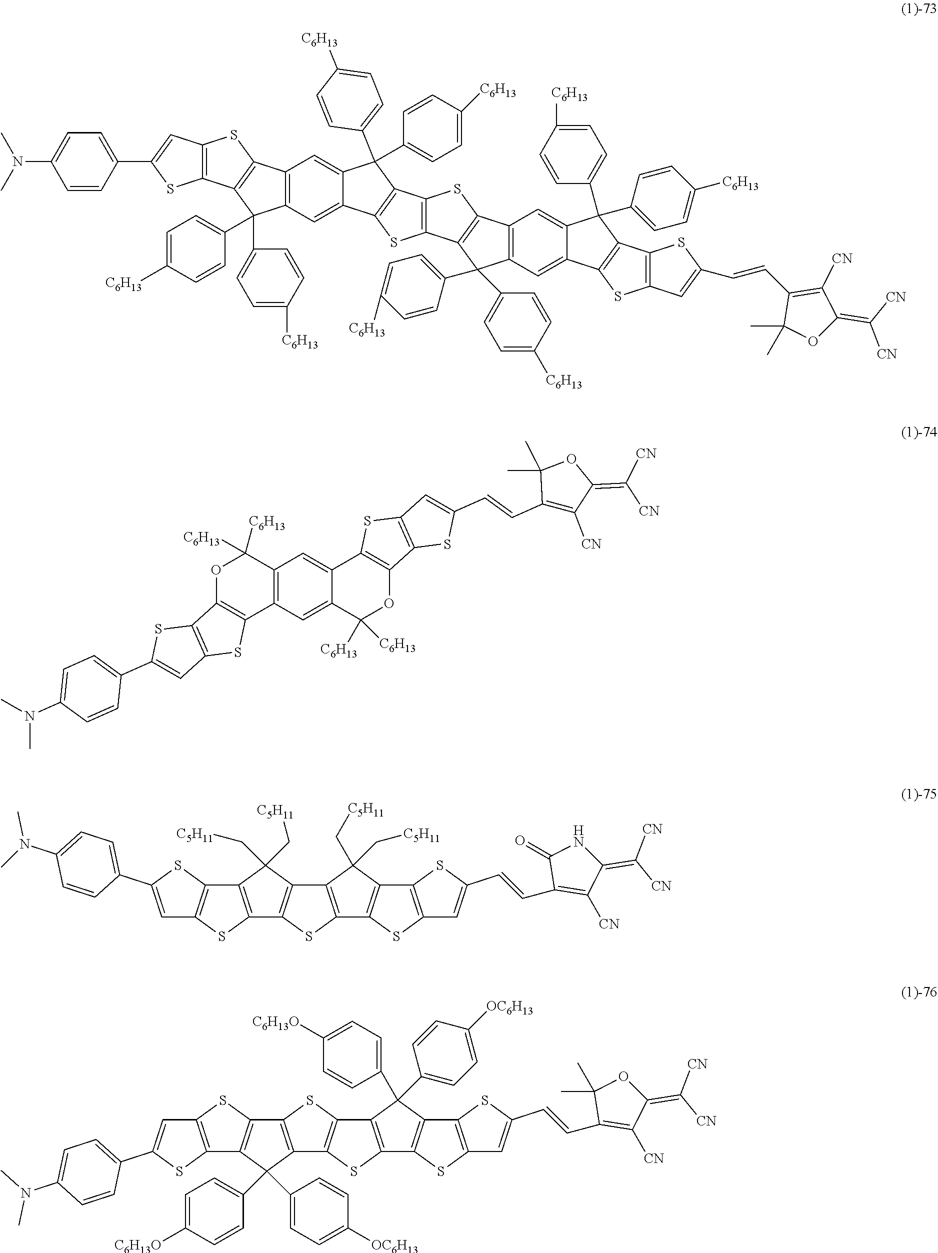

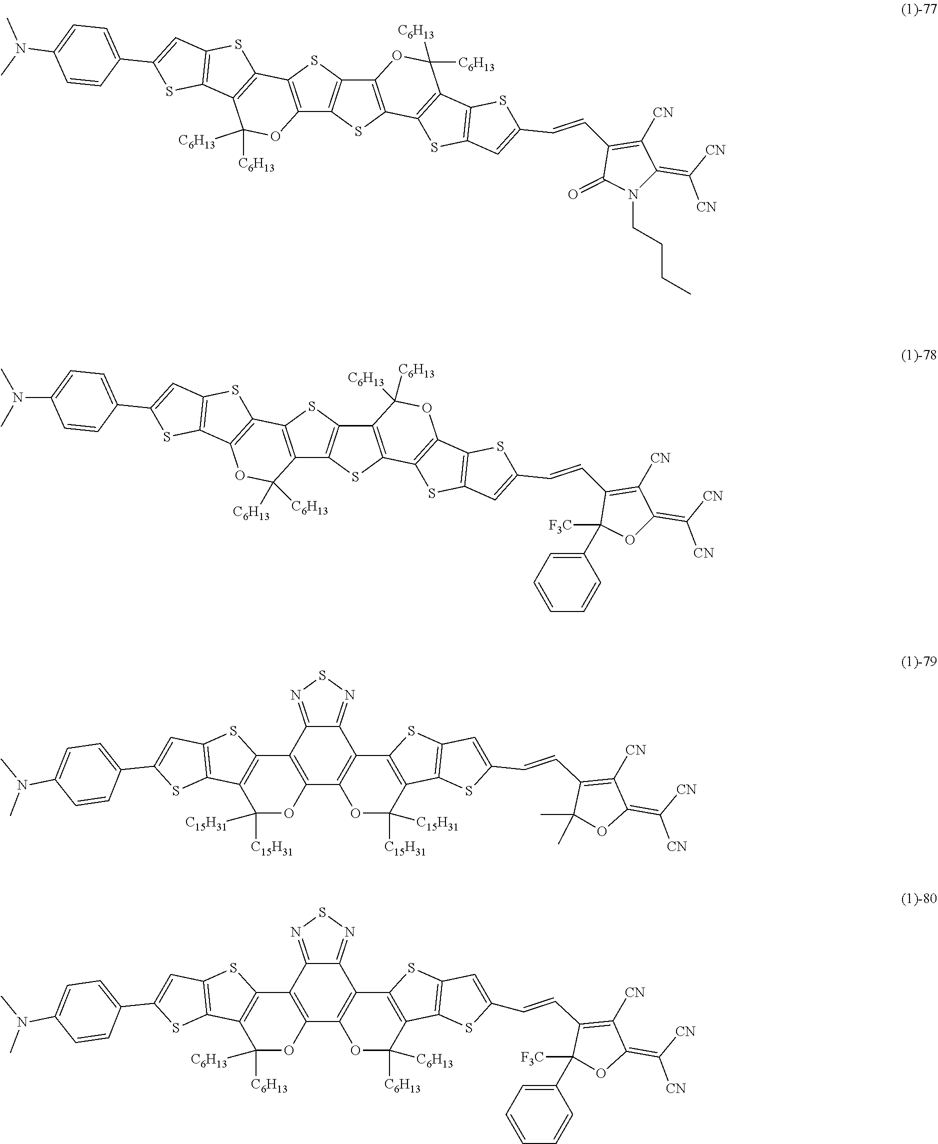

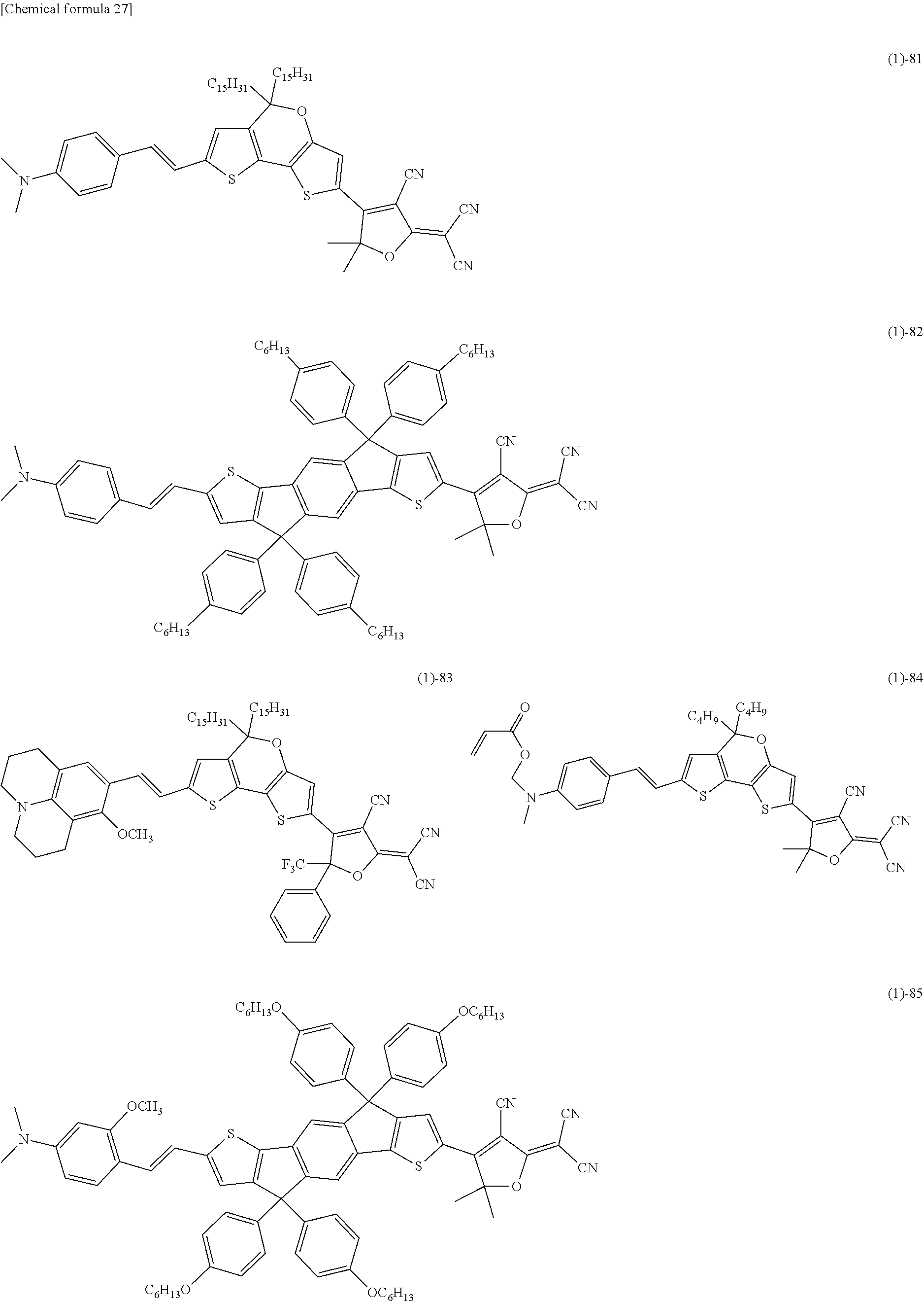

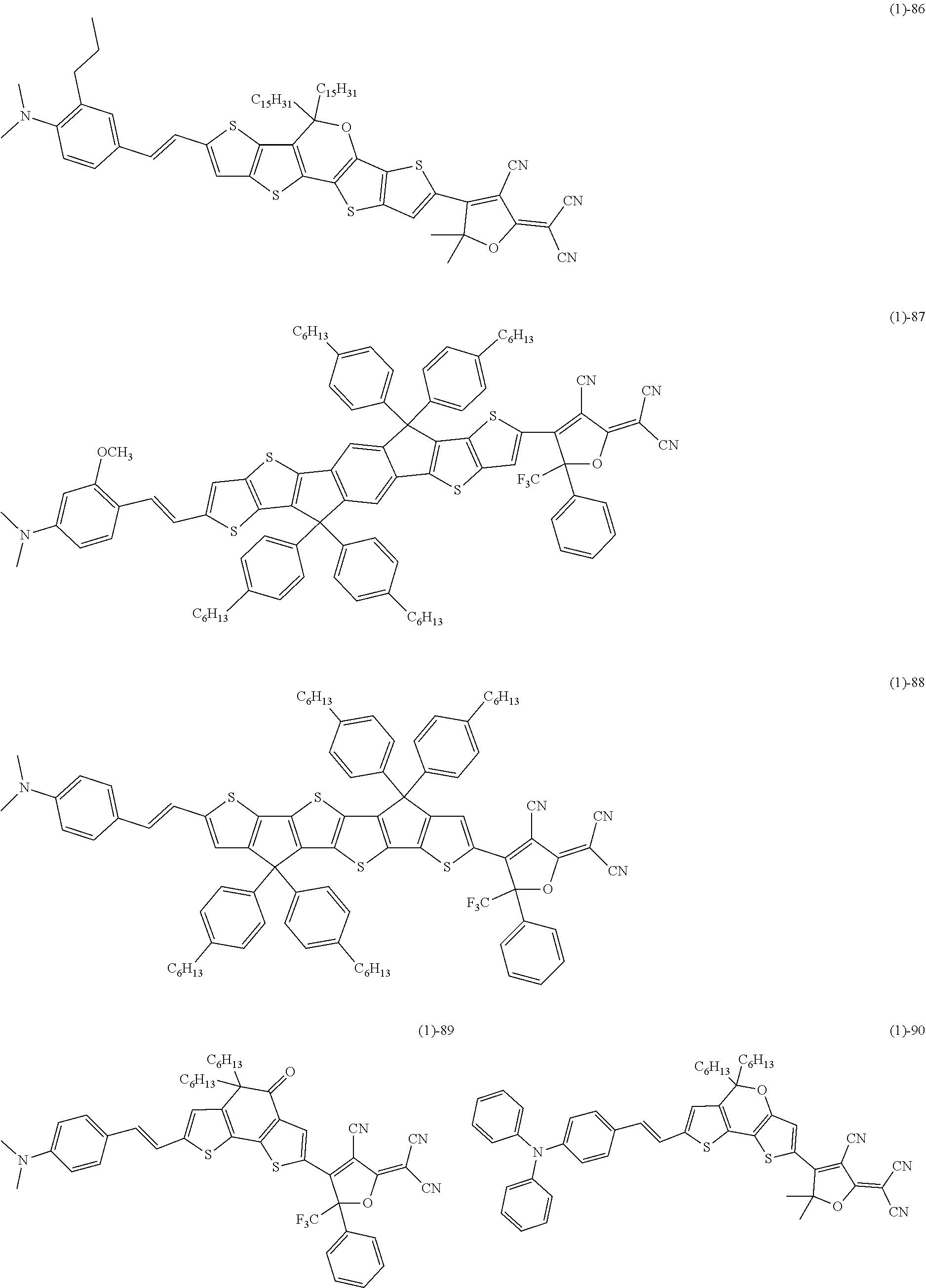

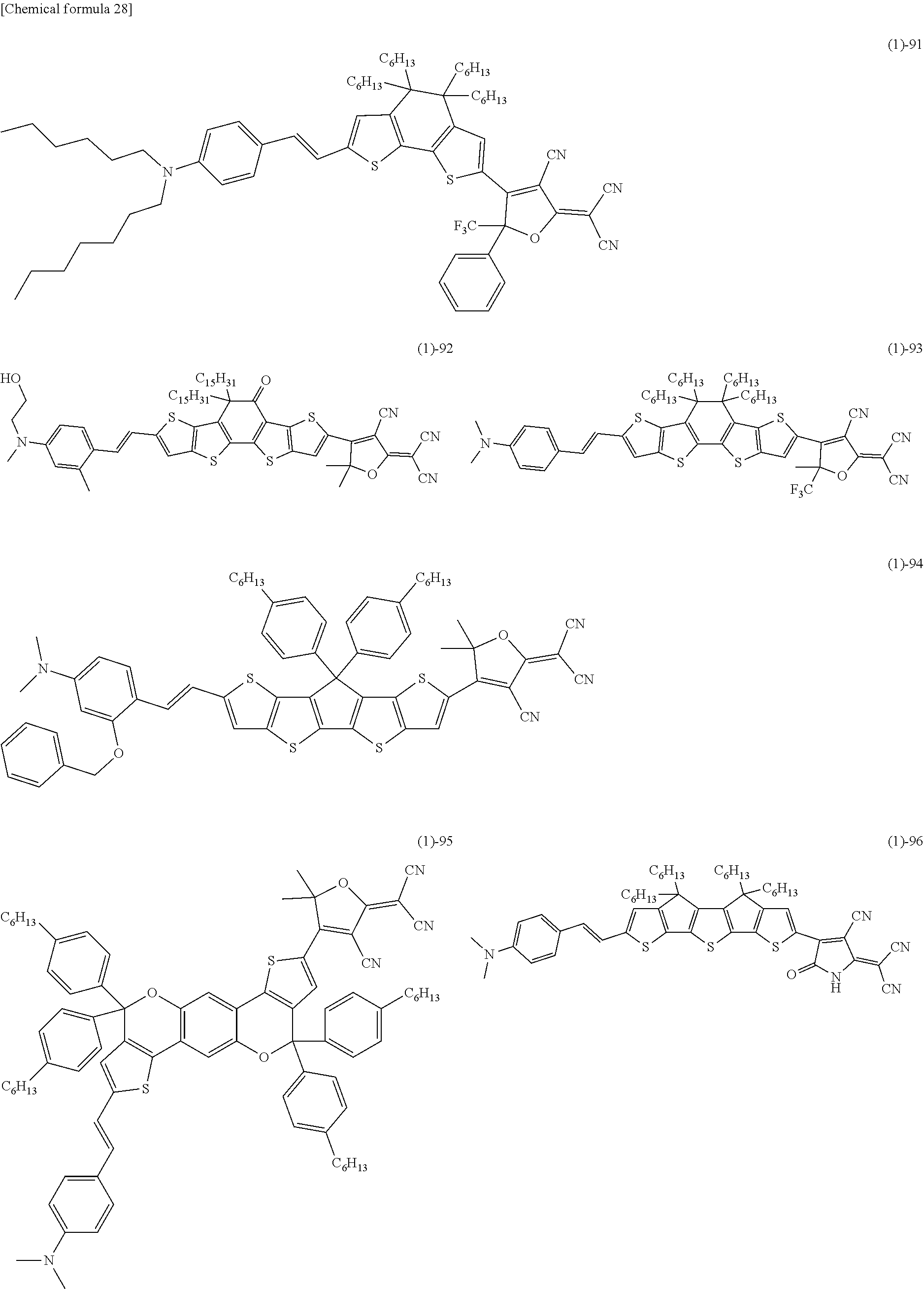

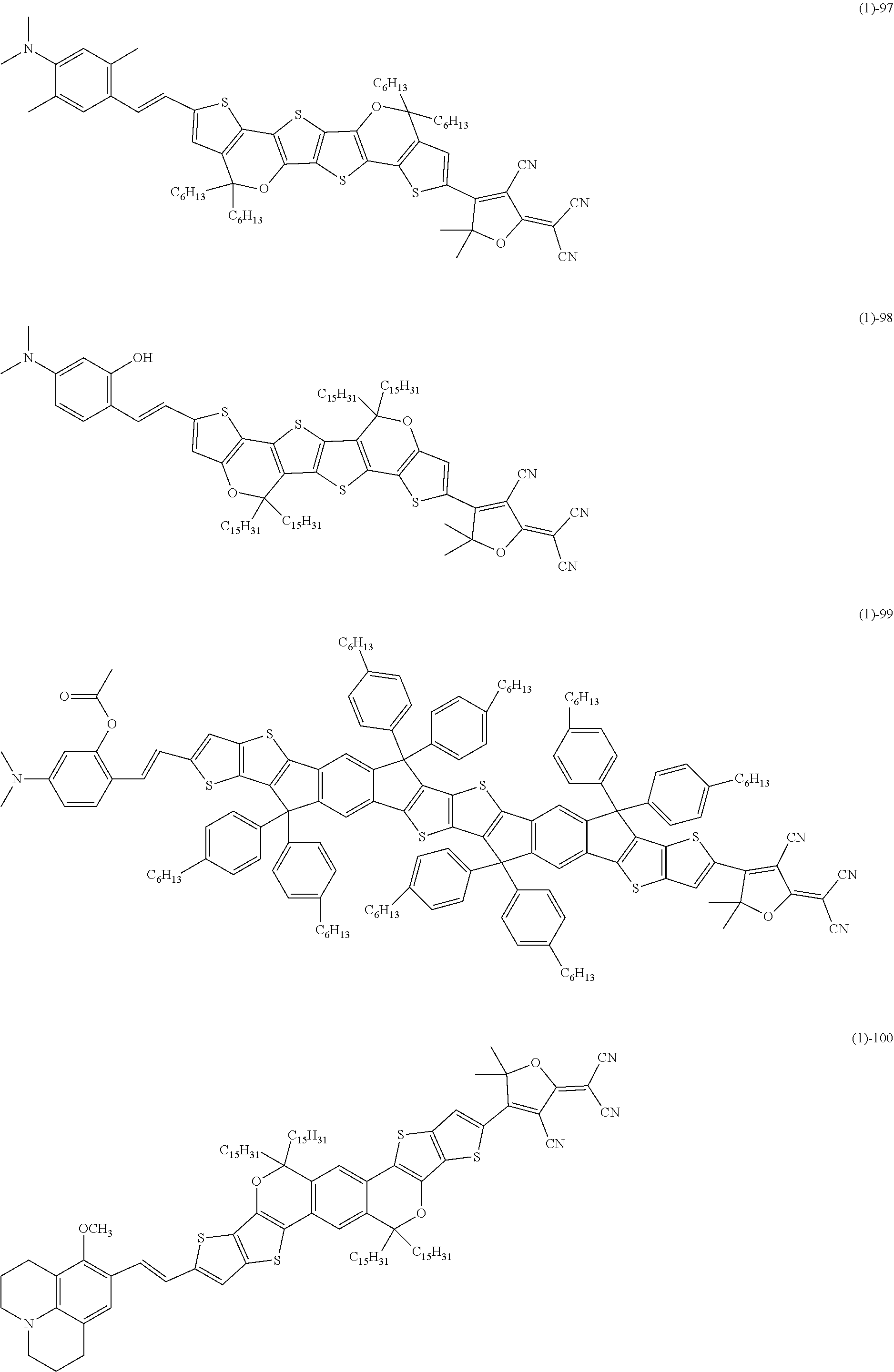

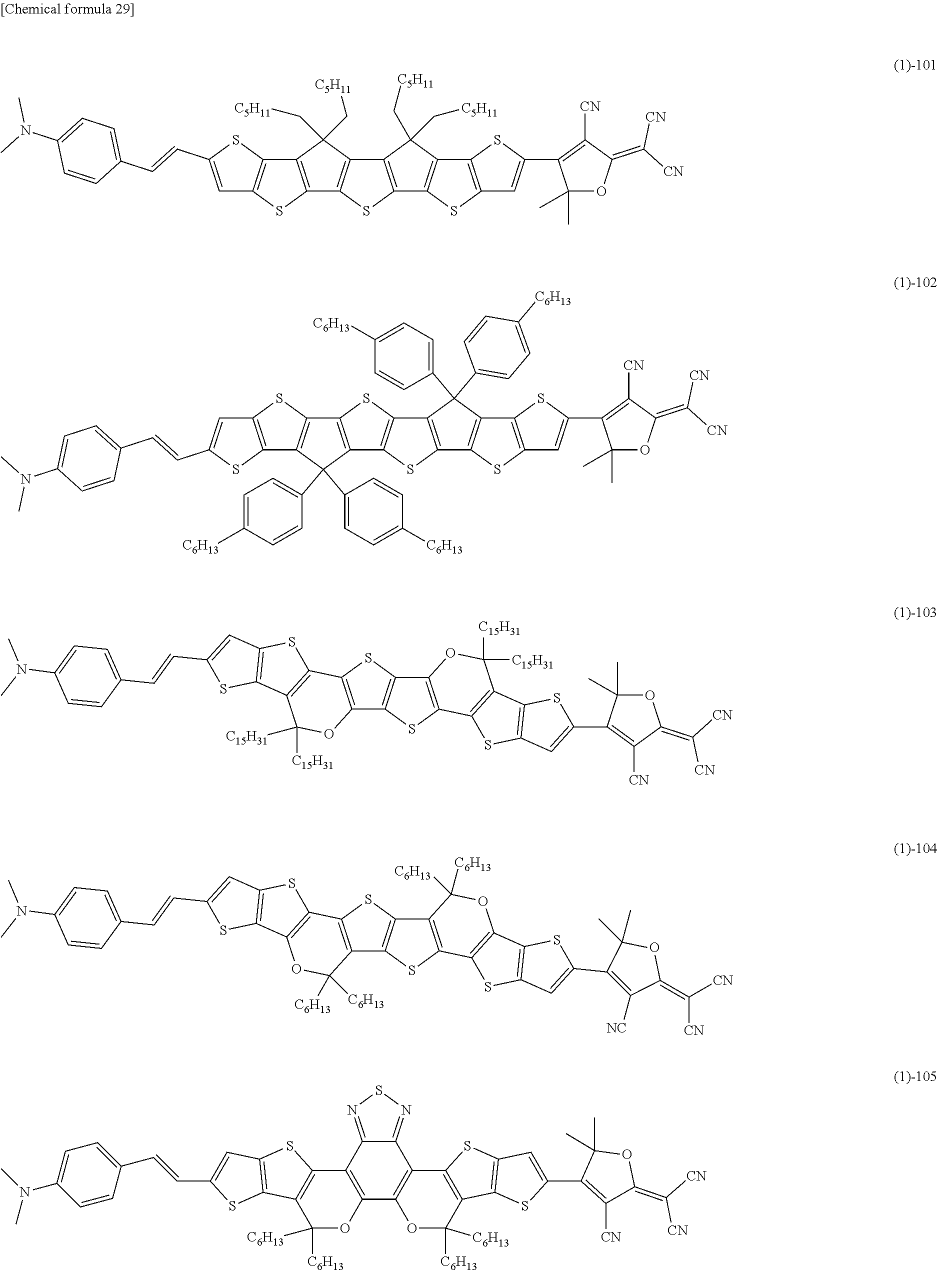

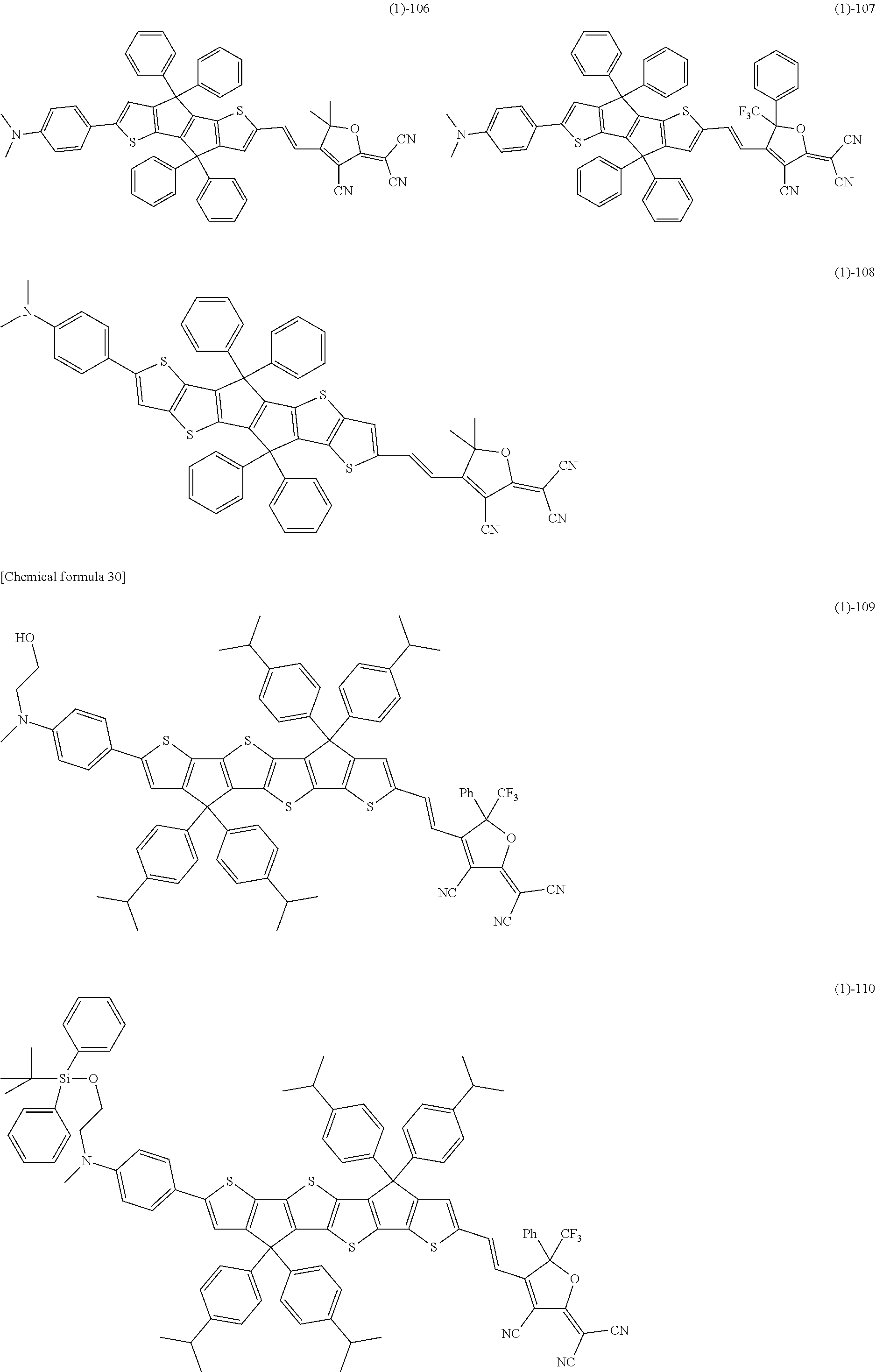

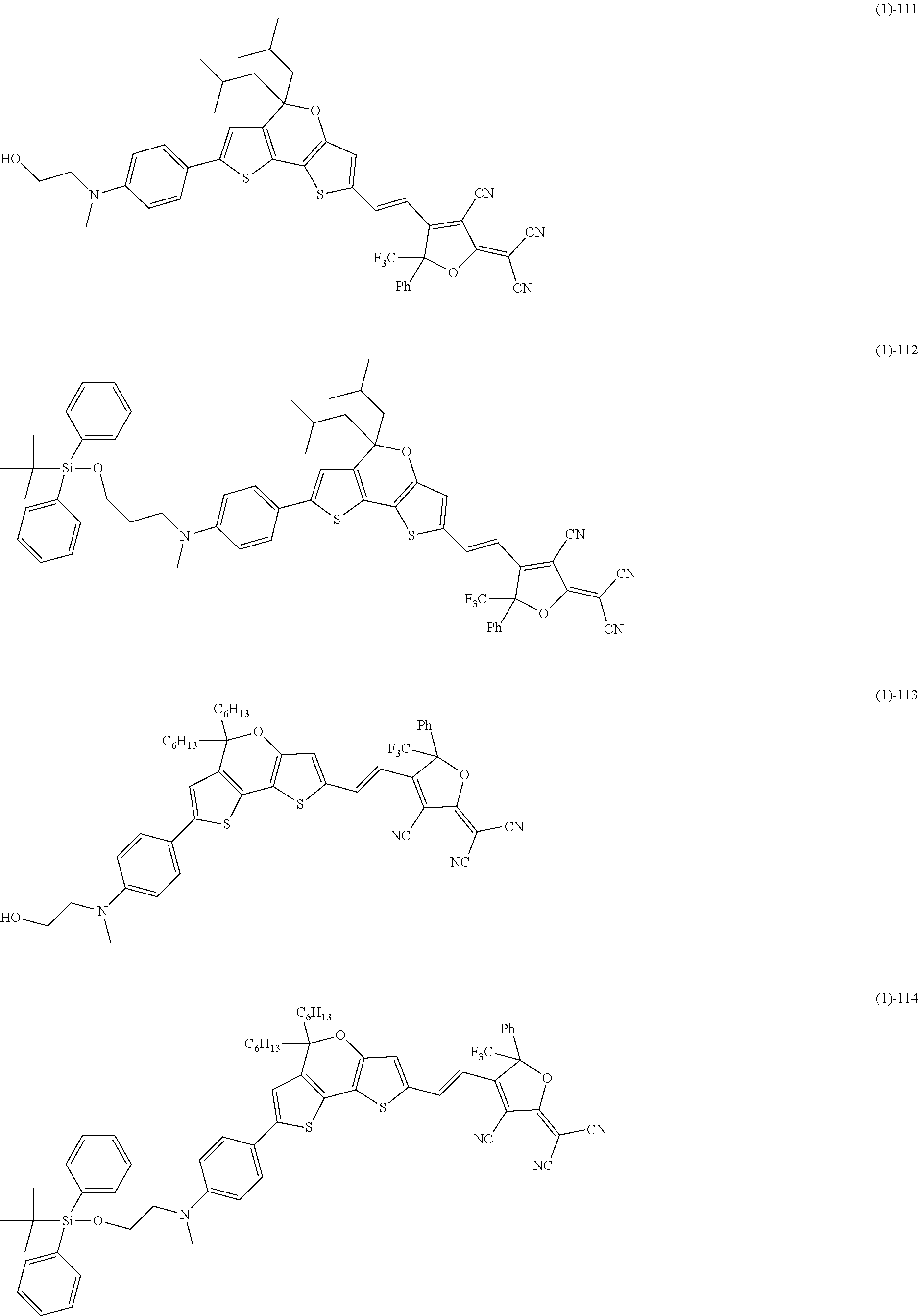

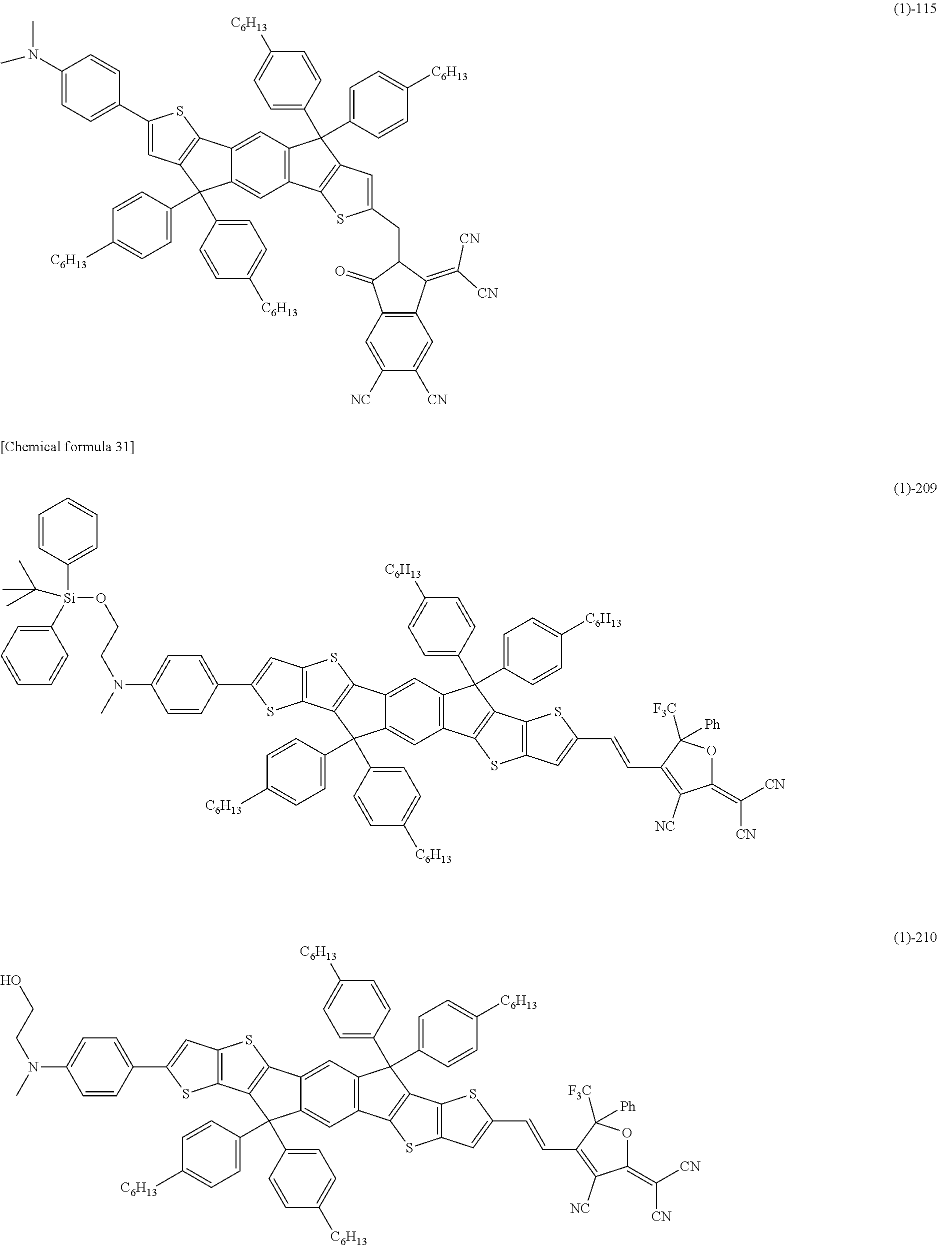

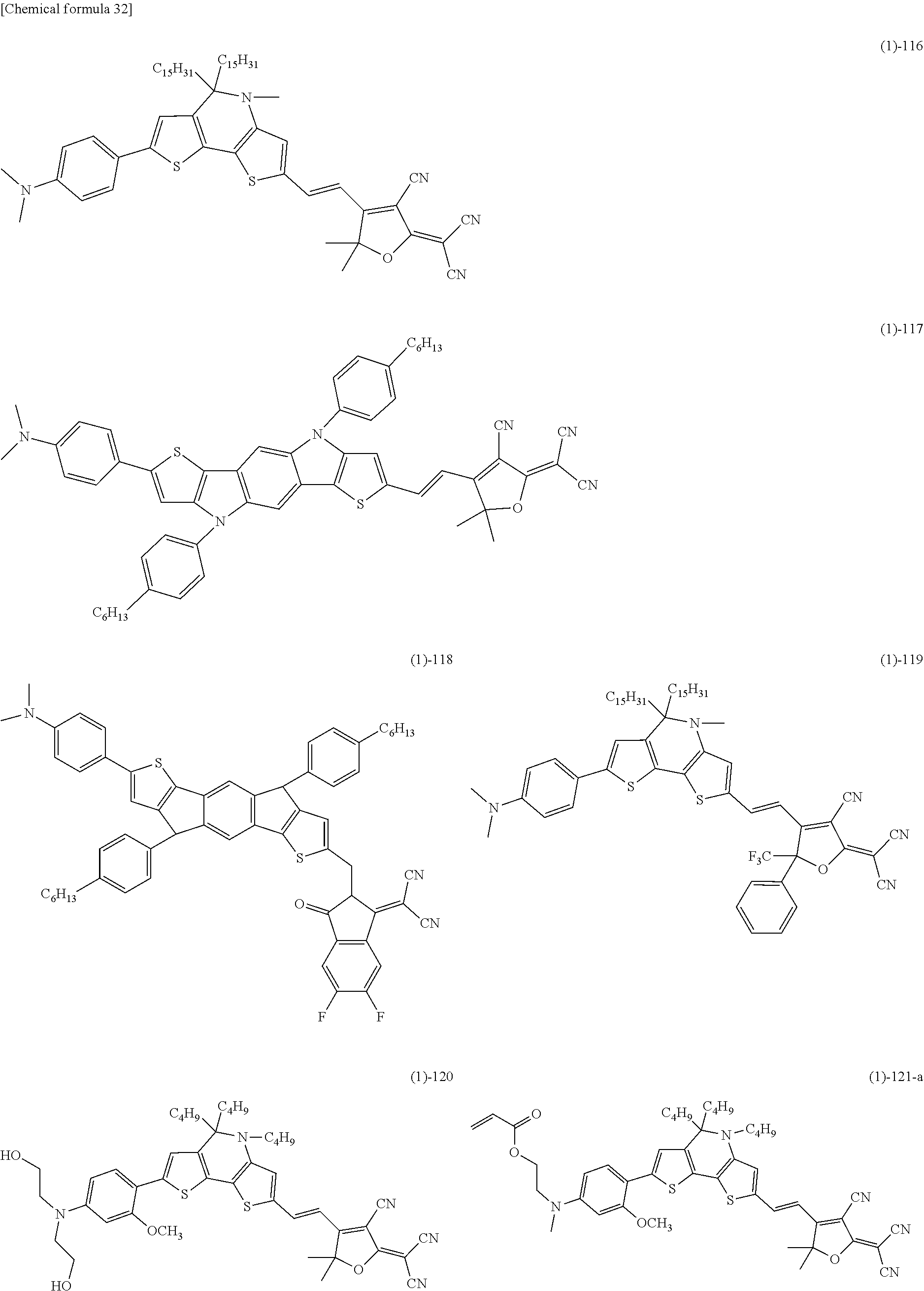

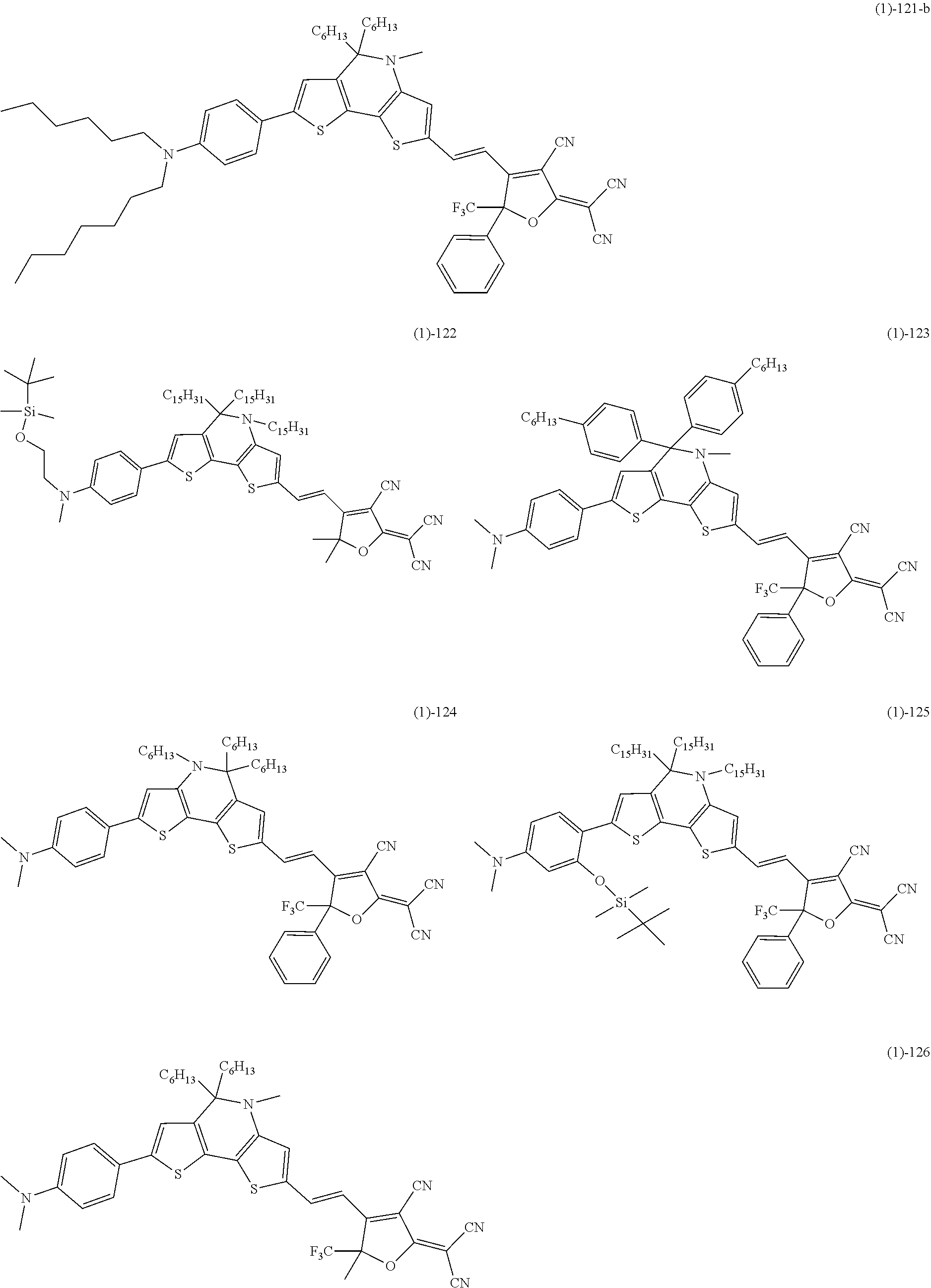

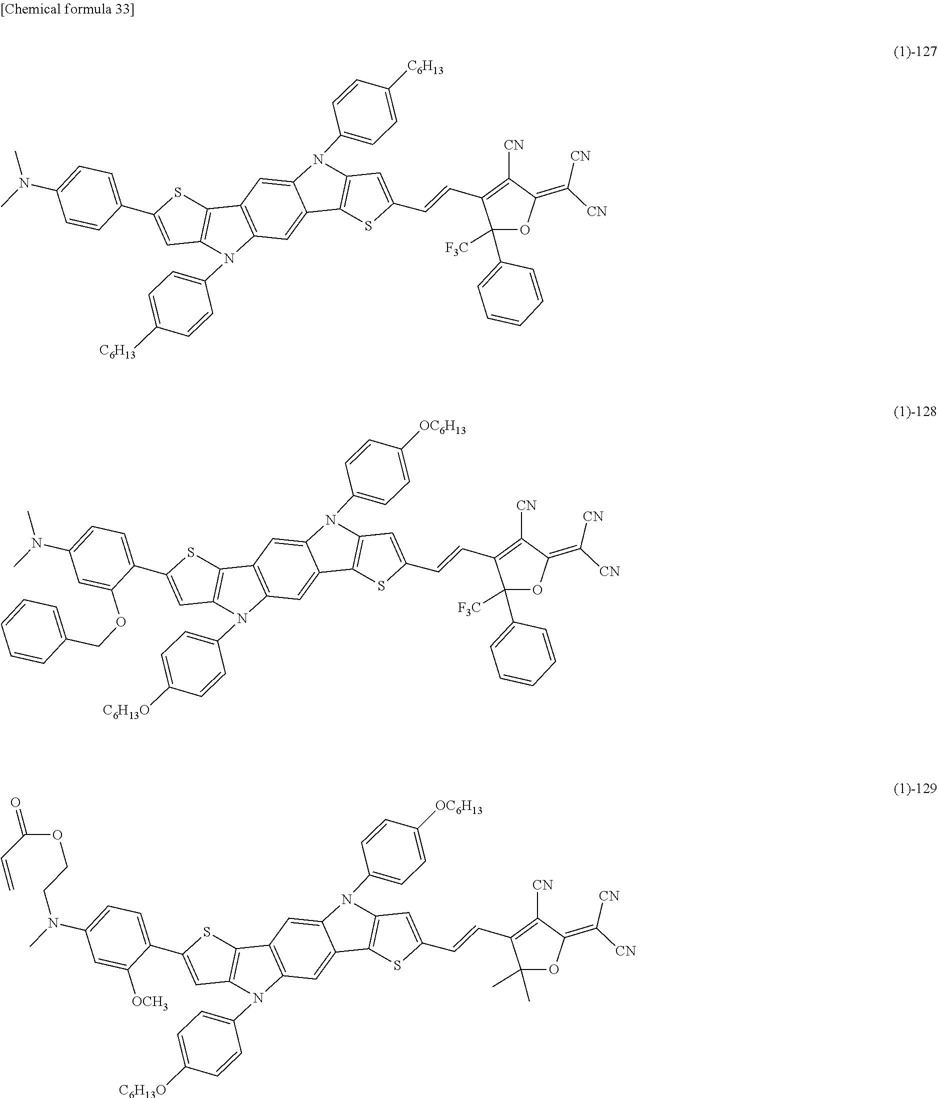

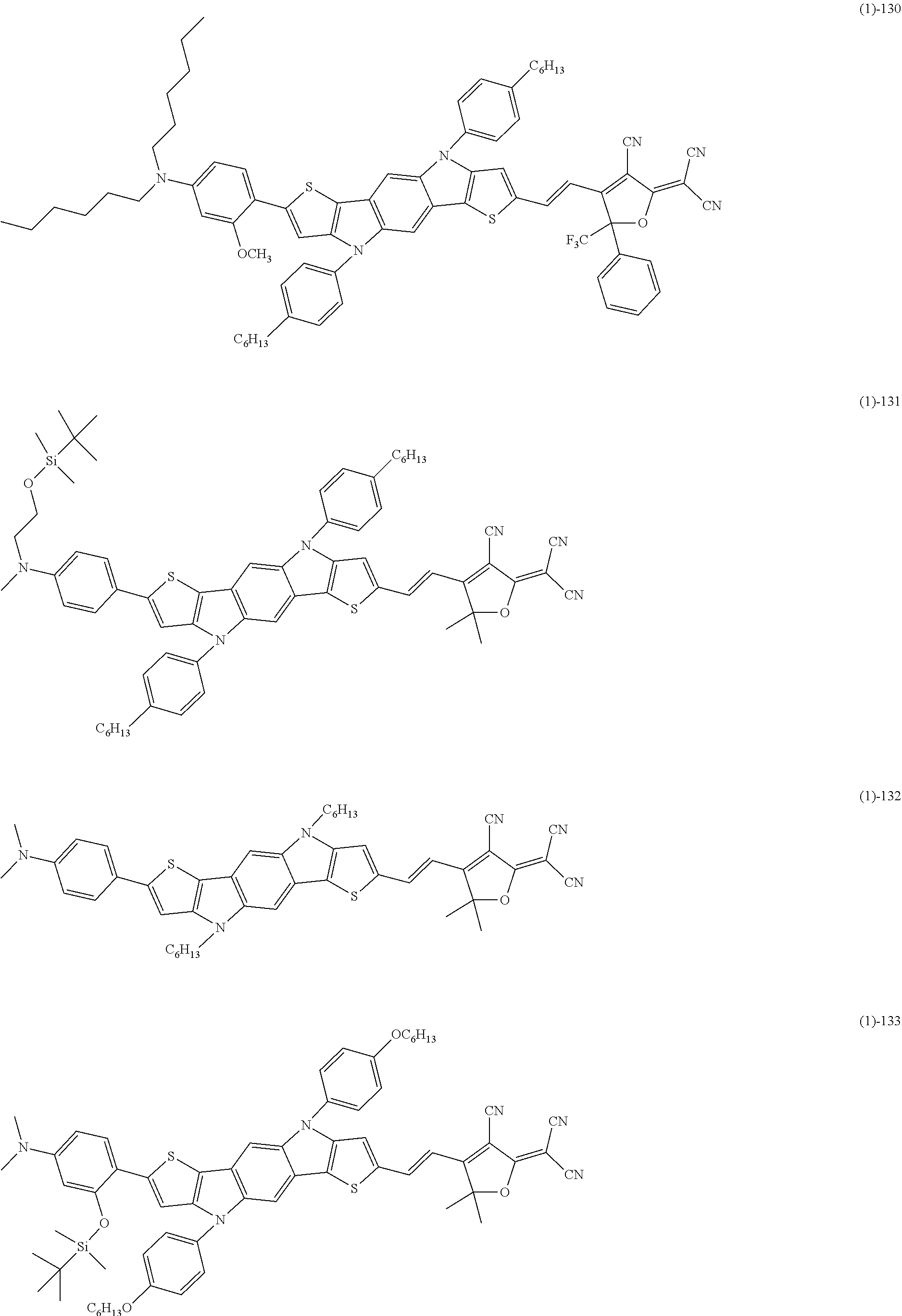

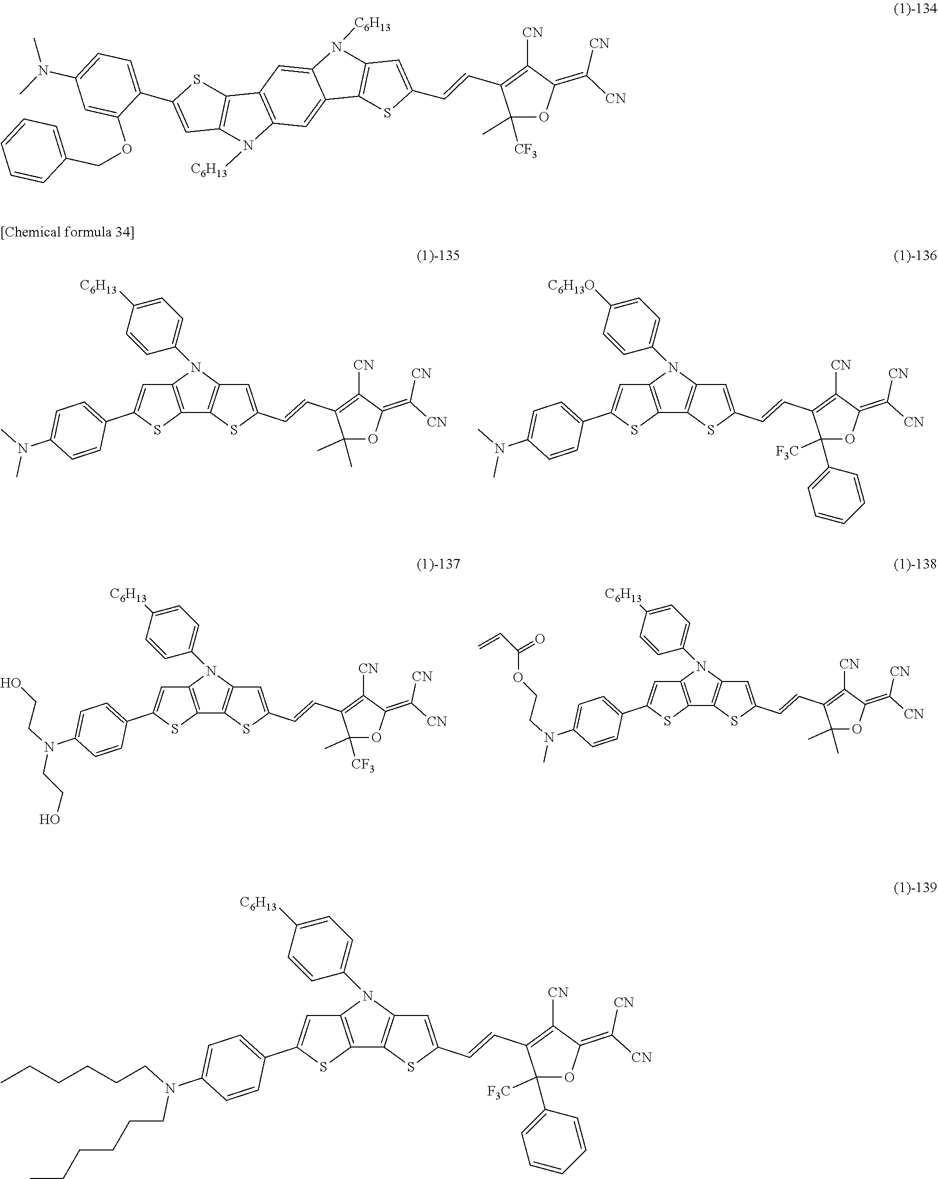

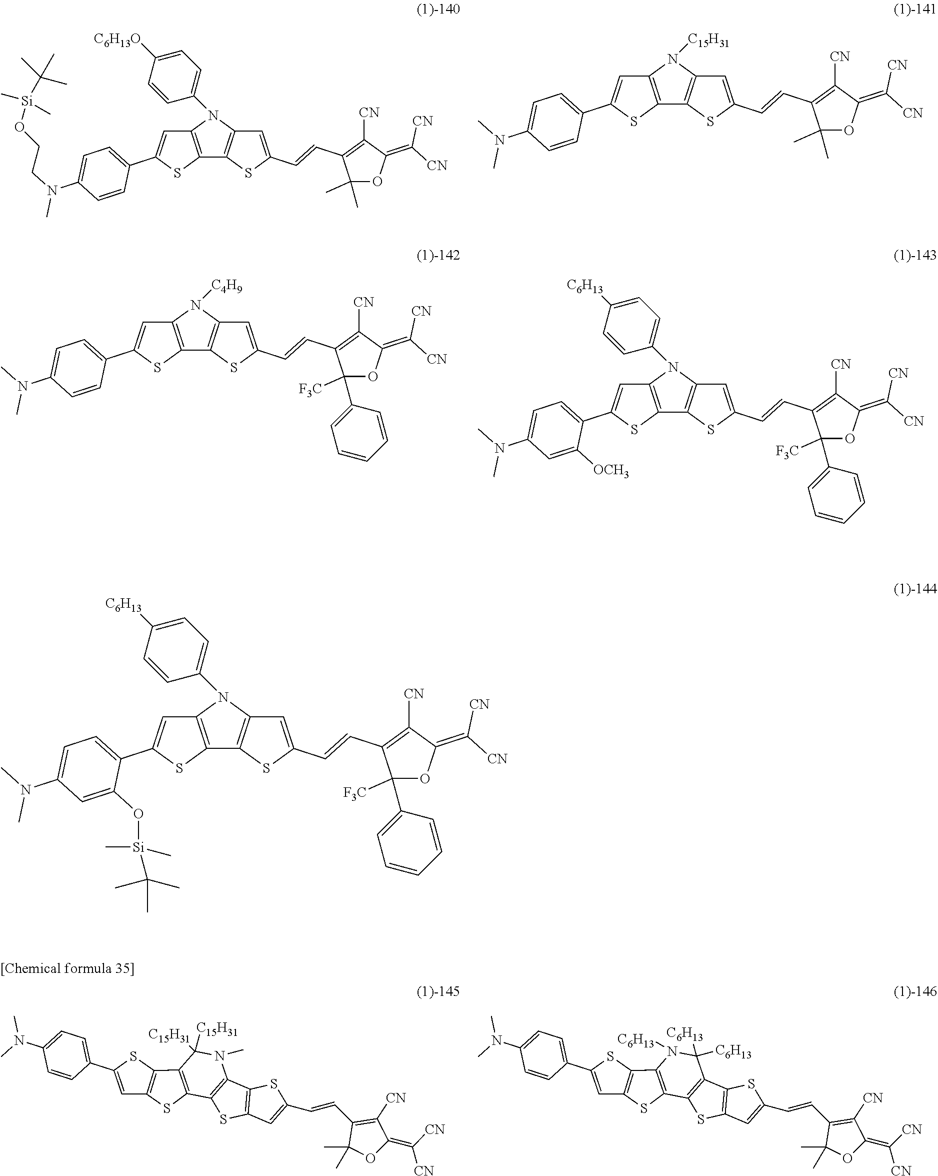

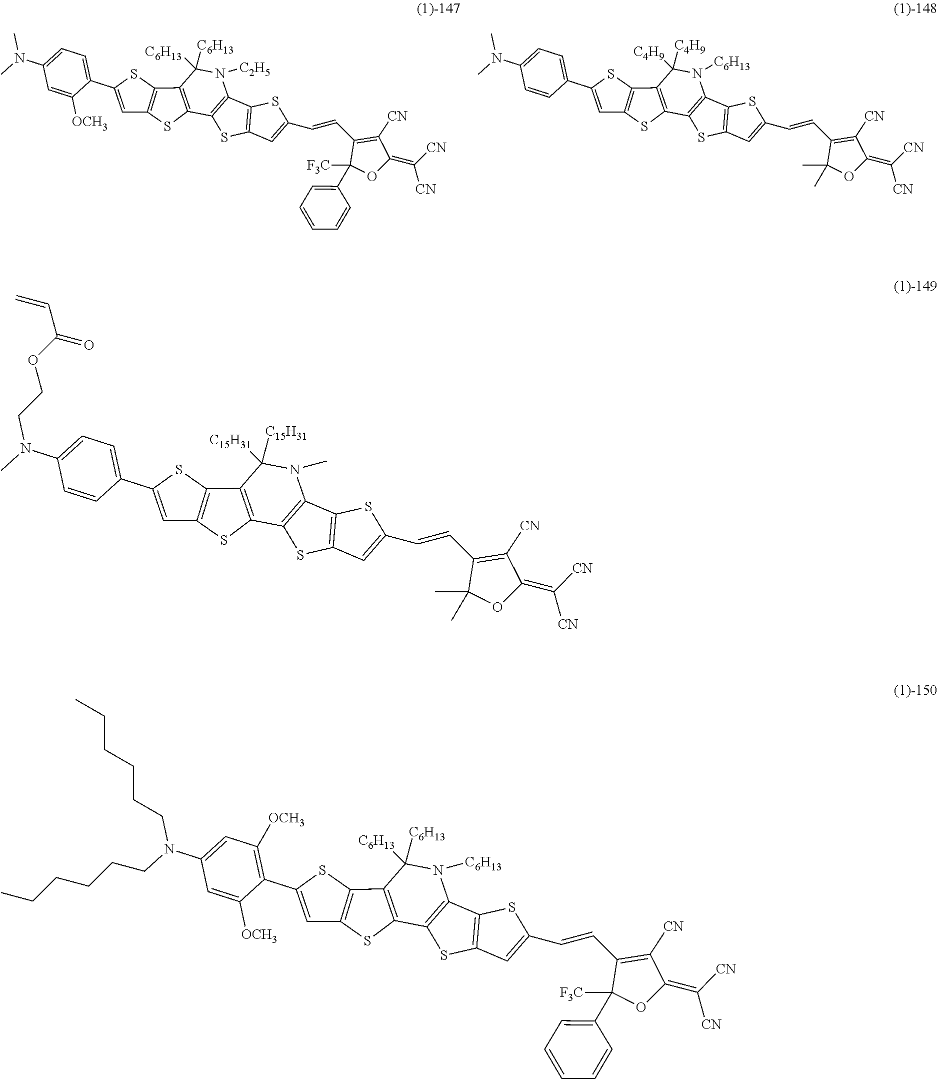

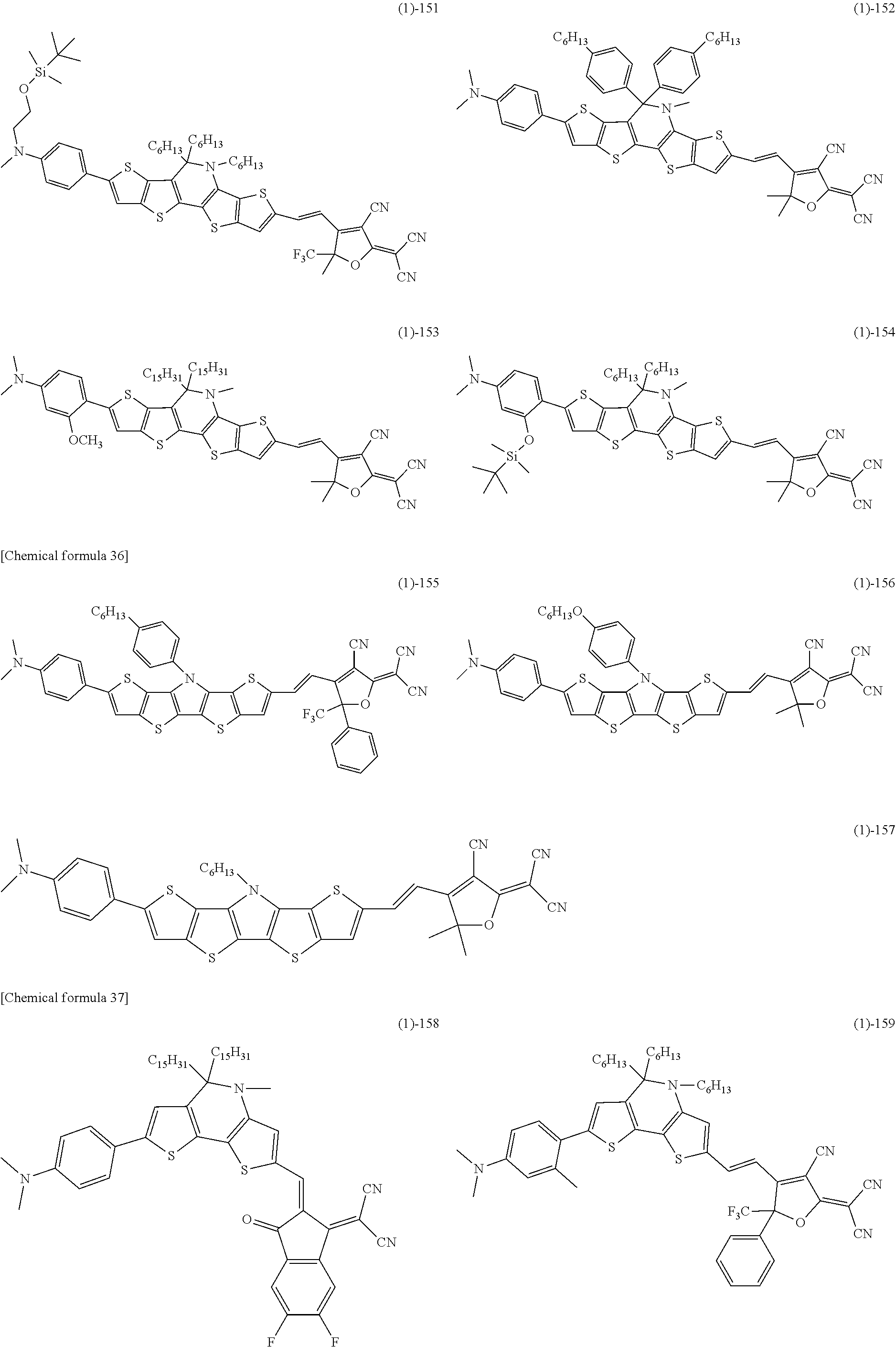

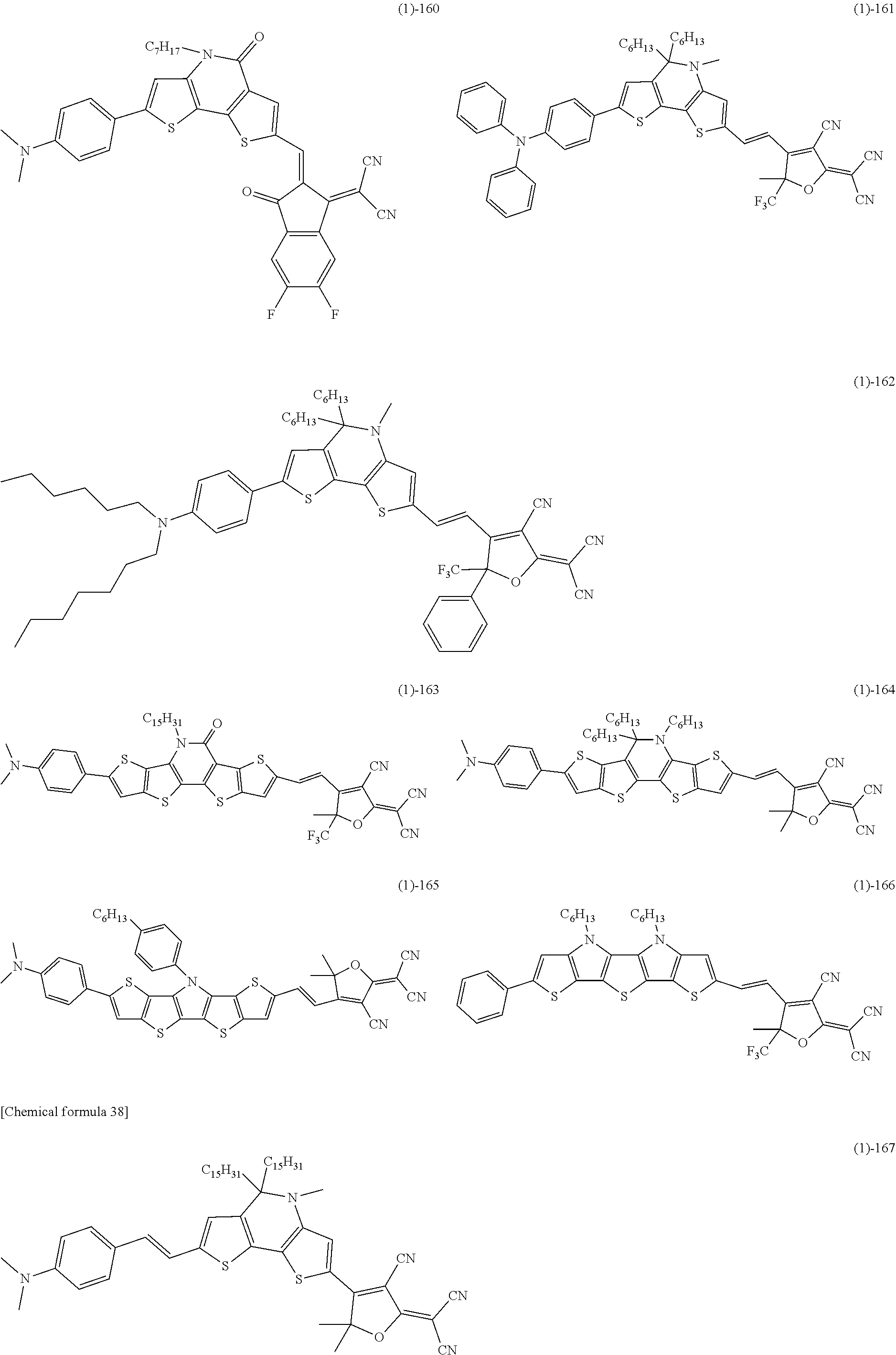

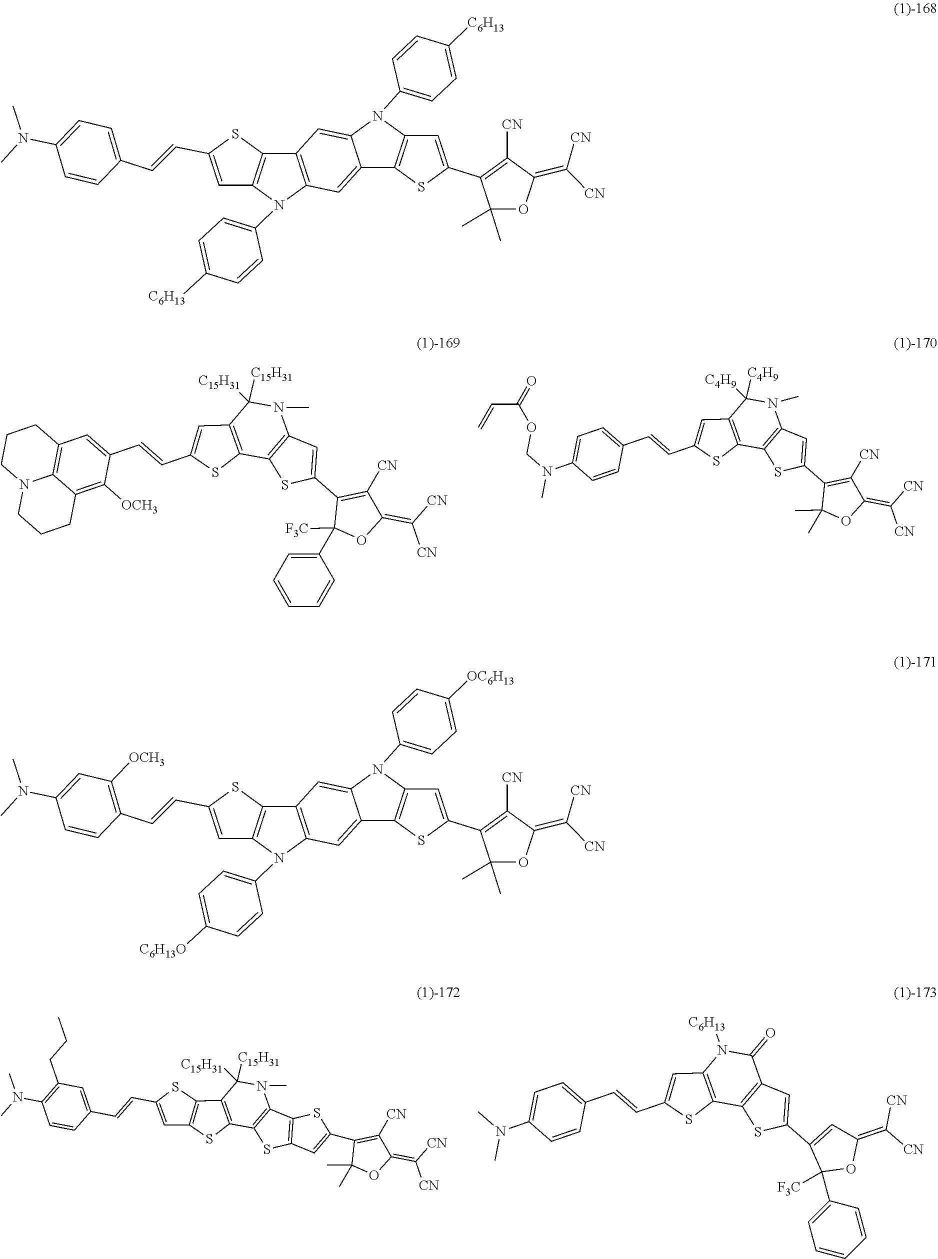

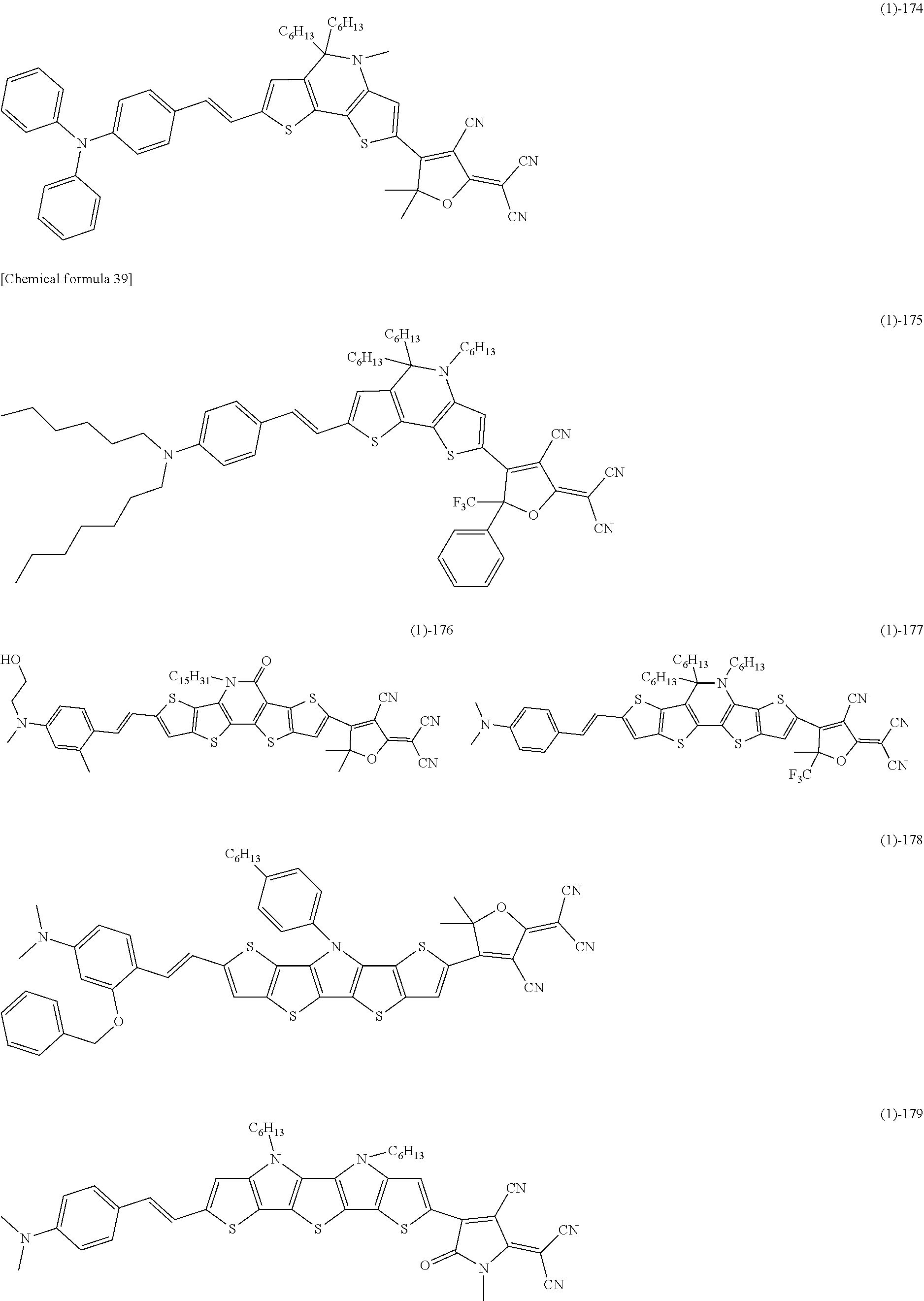

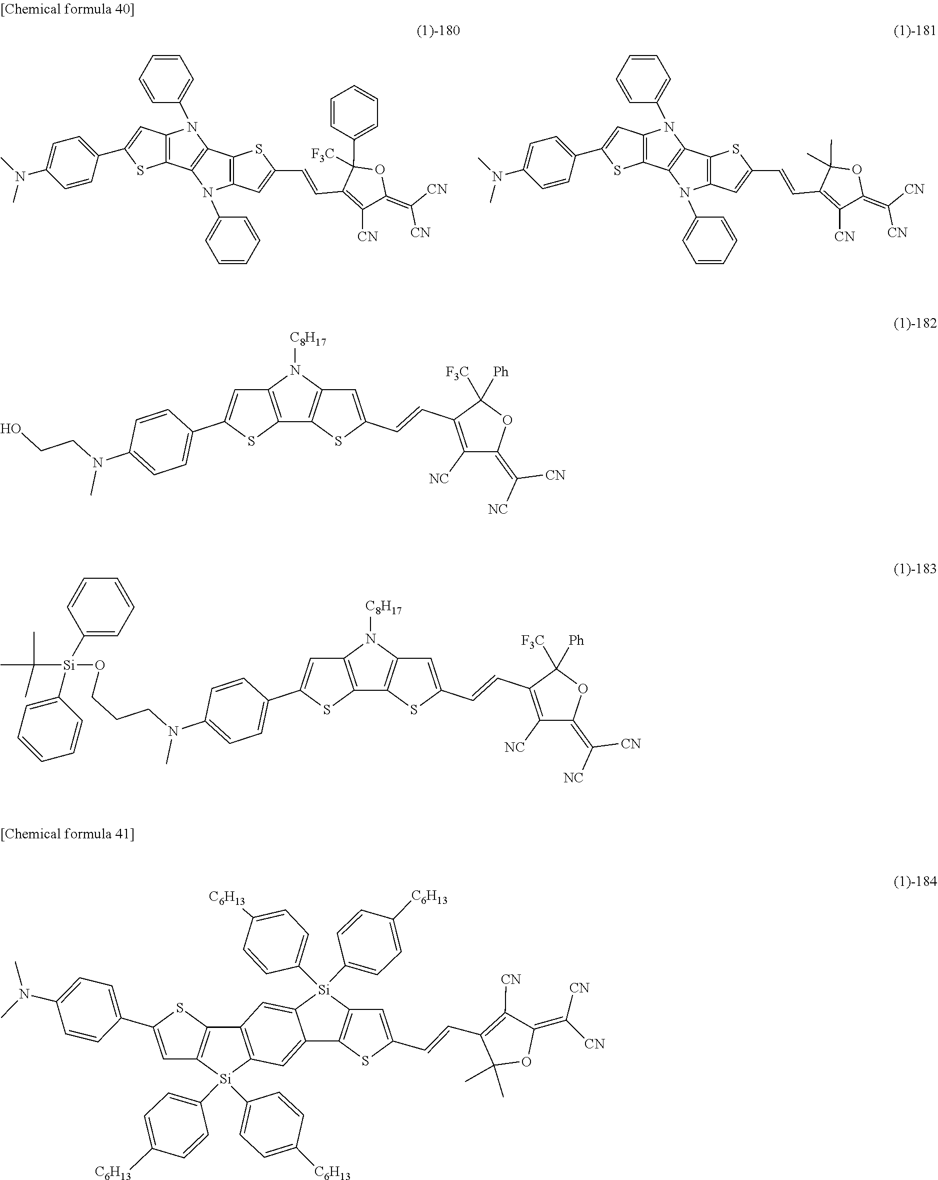

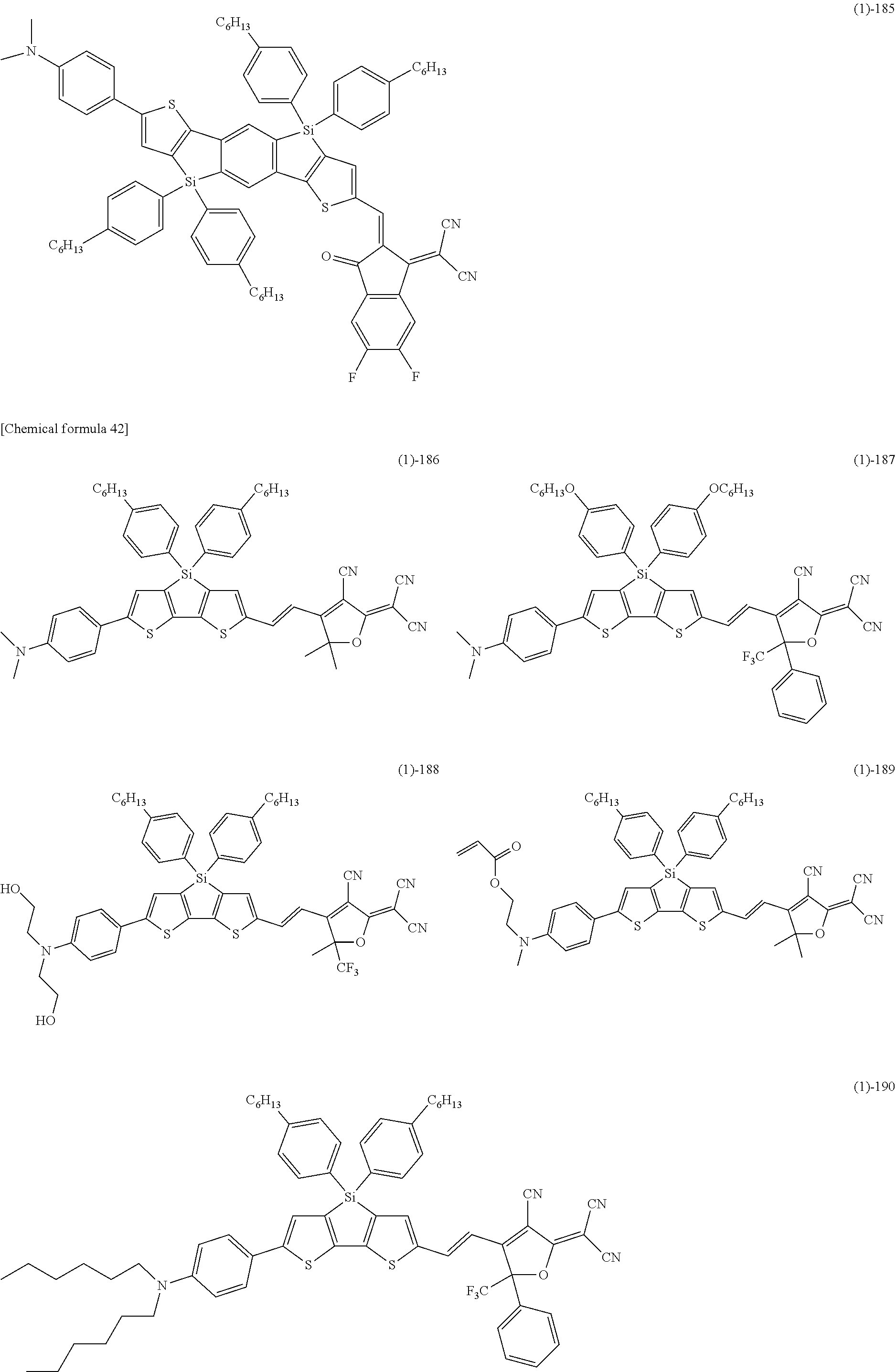

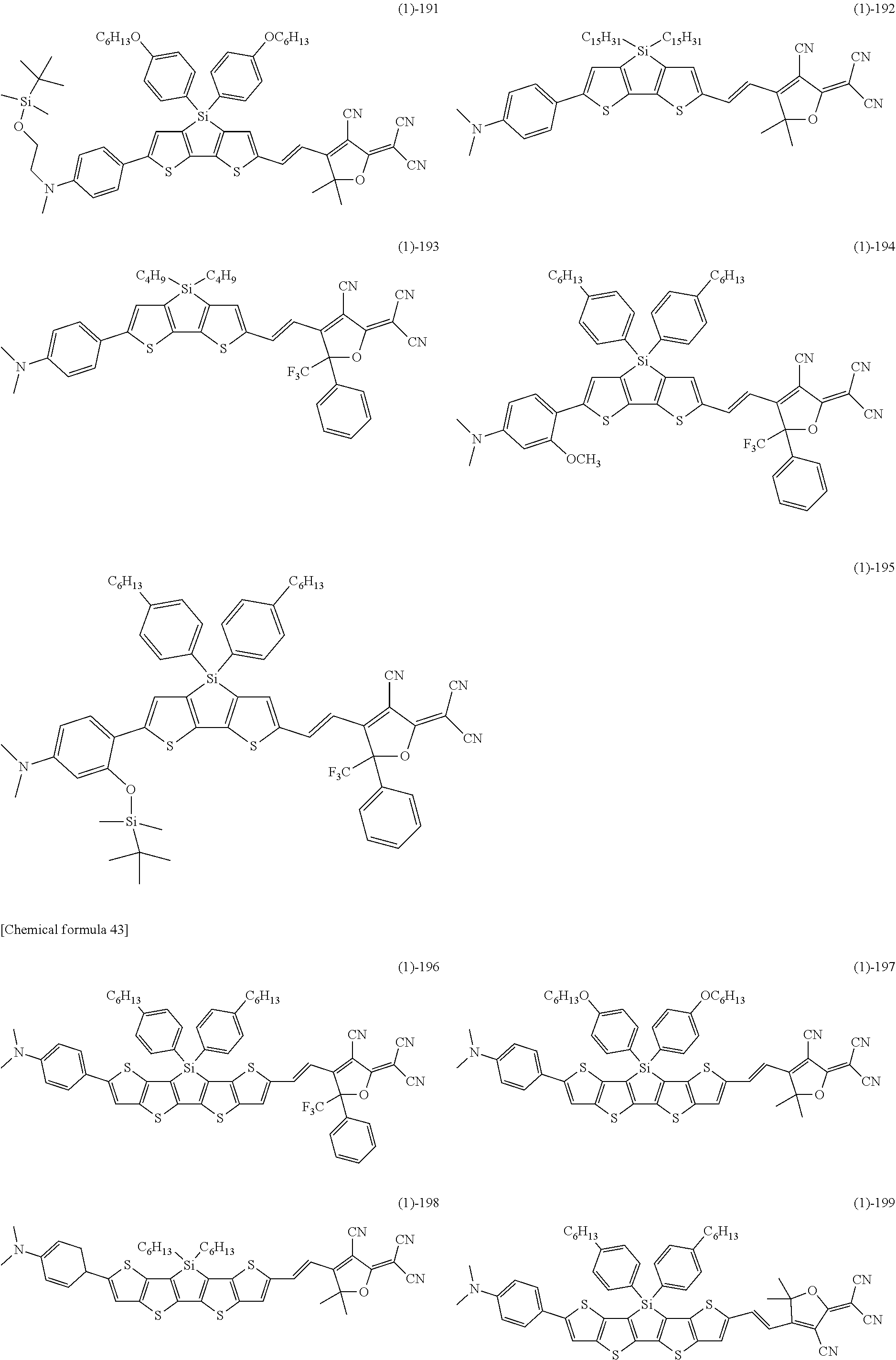

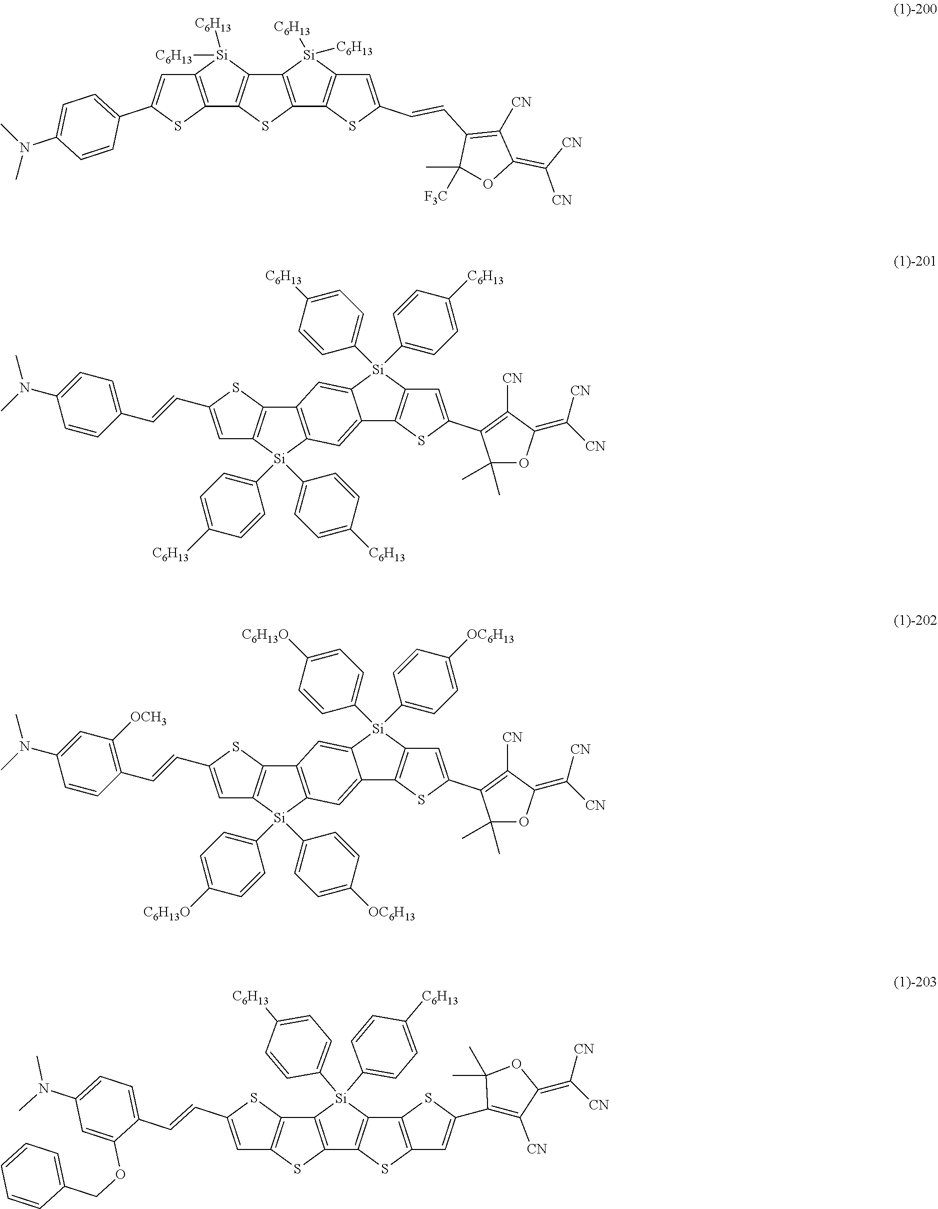

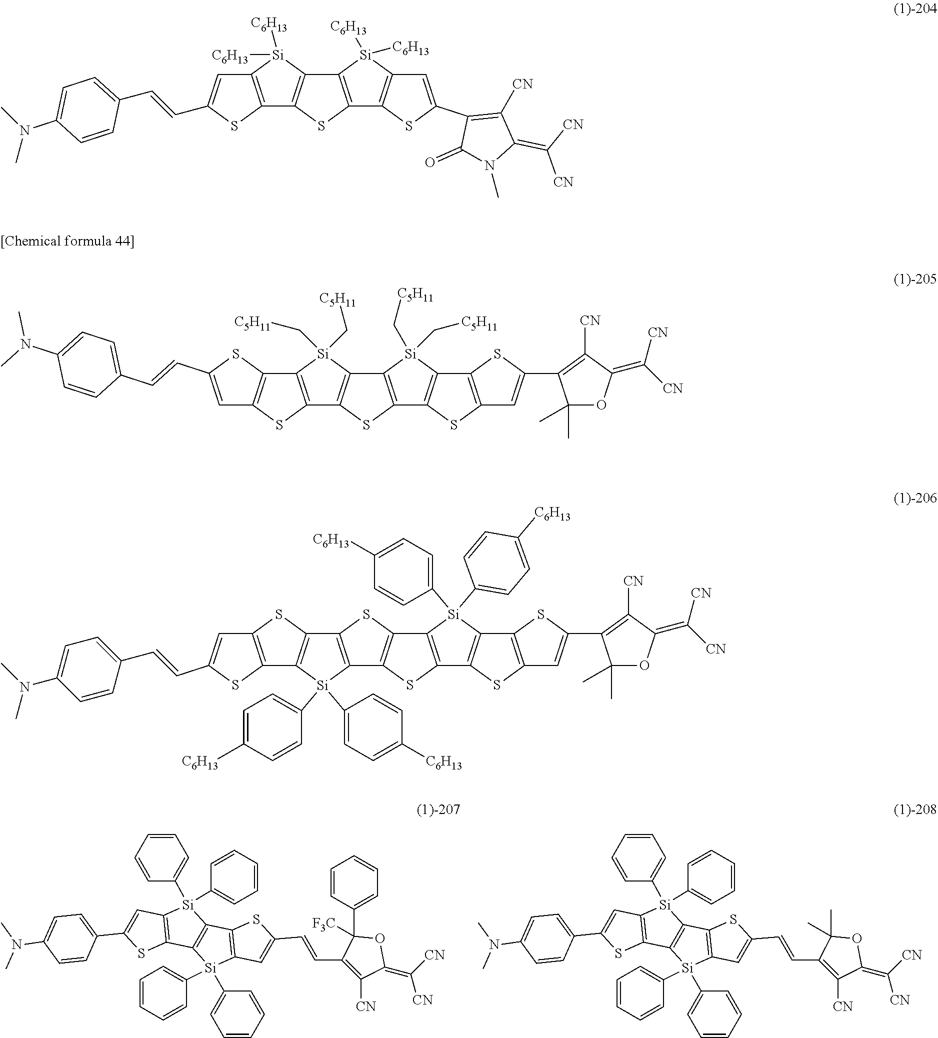

The compound represented by formula (1) is preferably a compound represented by any one of formulas (1)-1 to (1)-80, (1)-106 to (1)-194, (1)-209, and (1)-210, more preferably a compound represented by any one of formulas (1)-1, (1)-2, (1)-4 to (1)-60, (1)-106 to (1)-114, (1)-116, (1)-117, (1)-119 to (1)-194, (1)-209, and (1)-210, still more preferably a compound represented by any one of formulas (1)-1, (1)-2, (1)-4 to (1)-60, (1)-106, (1)-114, (1)-116, (1)-117, (1)-119 to (1)-194, (1)-209, and (1)-210, and particularly preferably a compound represented by any one of formulas (1)-1, (1)-2, (1)-4 to (1)-60, (1)-106, (1)-114, (1)-209, and (1)-210 because these compounds can suppress a multimerization reaction (for example, a Diels-Alder reaction) between the molecules by heating, and has excellent heat resistance.

The compound of the present embodiment (compound represented by formula (1)) may have a cis-trans isomer. In the compound of the present embodiment, generation of a trans isomer tends to be dominant, but any one of a cis isomer, a trans isomer, and a cis-trans isomer mixture can be used. Among these, the compound of the present embodiment is preferably a trans isomer from a viewpoint of easily ensuring a polarizability.

In the compound of the present embodiment (compound represented by formula (1)), the amount of change in maximum absorption wavelength Amax is small before and after a heat treatment (for example, about 200° C.). The small amount of change in maximum absorption wavelength Amax before and after the heat treatment means that the structure of the compound is maintained before and after the heat treatment, and heat resistance is high. The amount of change in maximum absorption wavelength Amax of the compound of the present embodiment before and after the heat treatment ((maximum absorption wavelength Amax before heat treatment)−(maximum absorption wavelength Amax after heat treatment)) is usually −20 to +20 nm (within 20 nm in absolute value), preferably −10 to +10 nm (within 10 nm in absolute value), more preferably −5 to +5 nm (within 5 nm in absolute value), and still more preferably −2 to +5 nm.

The amount of change in maximum absorption wavelength Amax before and after the heat treatment can be obtained, for example, by the following method. First, a predetermined amount of a compound sample is placed on an aluminum pan. The temperature is raised from 25° C. at a rate of 10° C./min under a nitrogen atmosphere using a thermogravimeter-differential thermal analyzer (TG-DTA), and is maintained for five minutes after reaching 200° C. After cooling, the compound sample is put into a screw tube together with the aluminum pan and eluted into chloroform (super dehydrated) to adjust the concentration to a predetermined concentration. Next, a UV visible light spectrum of the prepared solution is measured, and a maximum absorption wavelength Amax after the heat treatment is obtained. By subtracting this maximum absorption wavelength Amax from a maximum absorption wavelength Amax before the heat treatment separately obtained from measurement of a UV visible light spectrum of the compound sample before the heat treatment, the amount of change in maximum absorption wavelength Amax before and after the heat treatment can be obtained.

The compound of the present embodiment has excellent heat resistance while having a high hyperpolarizability. Therefore, therefore, by using the compound in production of an EO element, heat resistance required for a high-temperature process at poling, a thermal curing process for fixing film orientation, a high-temperature process at the time of mounting, and the like can be improved, and the degree of freedom in the process of element production can be increased.

### [Method for Producing Compound]

A method for producing the compound represented by formula (1) is not particularly limited. Here, the method for producing the compound represented by formula (1) will be described using a compound represented by formula (2b1-1) and a compound represented by formula (2a2) as examples.

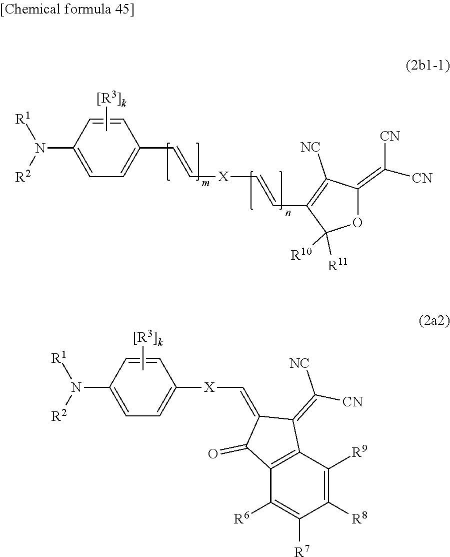

<Production Method A>

A production method A is a method for producing a compound represented by formula (2b1-1) in which m is 0 and n is 1.

The production method A may include, for example, a step of preparing a compound (x-1) (polycyclic condensed cyclic compound), a step of formylating the compound (x-1) by a Vilsmeier reaction to obtain a compound (x-2), a step of brominating the compound (x-2) to obtain a compound (x-3), a step of coupling the compound (x-3) and a compound (x-4) by Suzuki coupling to obtain a compound (x-5), and a step of performing aldol condensation of the compound (x-5) and a compound (x-6) to obtain a compound represented by formula (3b1-1). By such a production method A, a compound represented by formula (2b1-1) in which m is 0 and n is 1 (compound represented by formula (2b1-1a)) can be obtained.

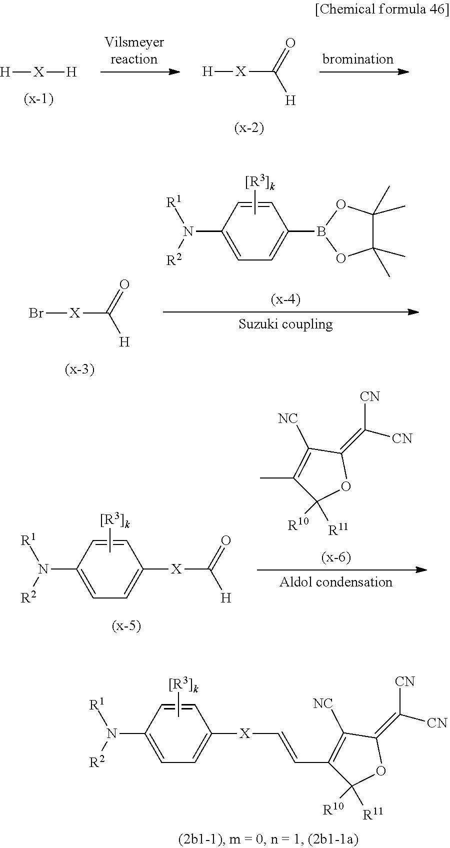

<Production Method B>

A production method B is a method for producing a compound represented by formula (3b1-1) in which m is 1 and n is 0.

The production method B includes, for example, a step of preparing a compound (y-1) (polycyclic condensed cyclic compound), a step of formylating the compound (y-1) by a Vilsmeier reaction to obtain a compound (y-2), a step of brominating the compound (y-2) to obtain a compound (y-3), a step of subjecting the compound (y-3) and a compound (y-4) to a Wittig reaction (Horner-Wadsworth-Emmons reaction) to obtain a compound (y-5), a step of converting a bromo group of the compound (y-5) to an organometallic reactant with magnesium or the like, causing the organometallic reactant to react with a compound (y-6), and then performing hydrolysis to obtain a compound (y-7), and a step of subjecting the compound (y-7) and a compound (y-8) to Knoevenagel condensation to obtain a compound represented by formula (3b1-1). By such a production method B, the compound represented by formula (3b1-1) in which m is 1 and n is 0 can be obtained.

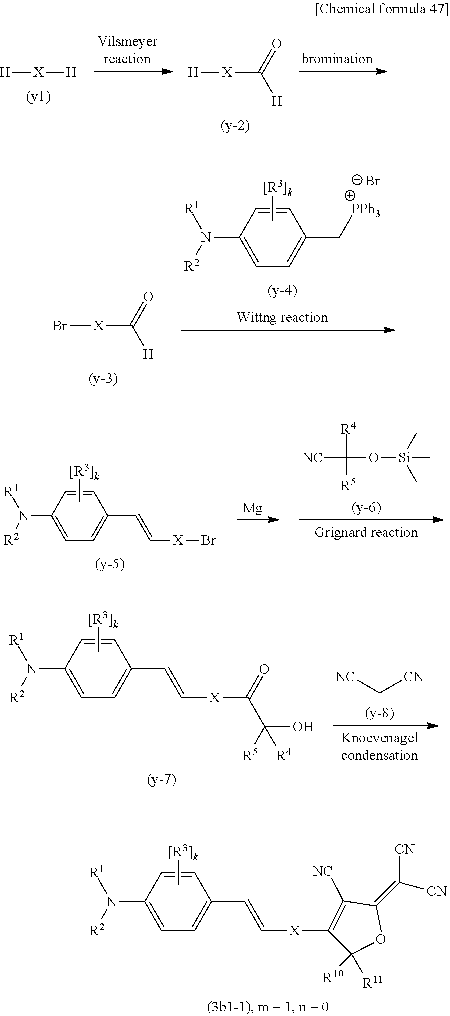

<Production Method C>

A production method C is a method for producing a compound represented by formula (2a2).

The production method C may include, for example, a step of obtaining the compound (x-5) in a similar manner to the production method A, and a step of performing aldol condensation of the compound (x-5) and a compound (z-1) to obtain a compound represented by formula (2a2). By such a production method C, the compound represented by formula (2a2) can be obtained.

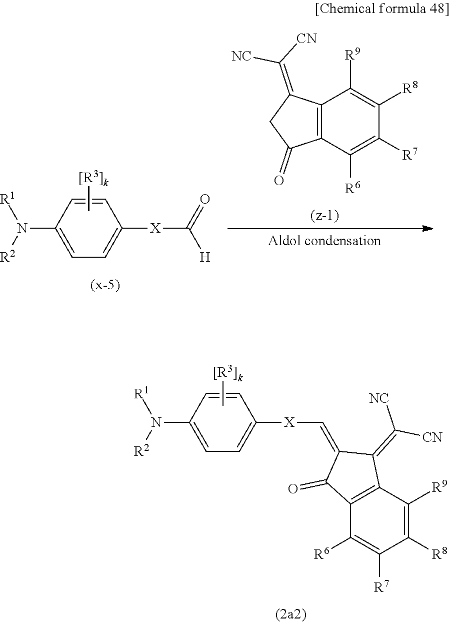

[Electrooptic Composition, Electrooptic Film, and Electrooptic Element]An EO composition, an EO film, and an EO element of the present embodiment can be produced by a known method (methods described in, for example, Oh et al., IEEE Journal of Selected Topics in Quantum Electronics, Vol. 7, No. 5, pp. 826-835, September/October 2001; Dalton et al., Journal of Materials Chemistry, 1999, 9, pp. 1905-1920; Toshikuni Kaino, Journal of the Institute of Electronics, Information and Communication Engineers, CVol. J84-C, No. 9, pp. 744-755, September 2001; and Ma et al., Advanced Materials, Vol. 14, No. 19, 2002, pp. 1339-1365).

The EO composition of the present embodiment contains the above EO compound. The EO composition of the present embodiment may further contain a host material capable of dispersing the compound. In order to exhibit excellent EO characteristics, it is important that the EO compound is uniformly dispersed at a high concentration in the host material. Therefore, the host material preferably exhibits high compatibility with the EO compound. The EO composition of the present embodiment can be suitably used for forming an EO film or forming an EO element. That is, the EO composition of the present embodiment may be a composition for forming an EO film or a composition for forming an EO element.

Examples of the host material include a resin such as a poly (meth)acrylate including polymethyl methacrylate (PMMA), polyimide, polycarbonate, polystyrene, polysulfone, polyether sulfone, a silicon-based resin, or an epoxy-based resin. These resins have excellent compatibility with the EO compound, and tend to have excellent transparency and moldability when being used as an EO element.

Examples of a method for dispersing the EO compound in the host material include a method for dispersing the EO compound and the host material in an organic solvent at an appropriate mixing ratio.

The host material may contain a resin having a reactive functional group capable of forming a covalent bond with the EO compound. Furthermore, at least a part of the EO compound is preferably bonded to the resin having the reactive functional group. By inclusion of such a host material, the EO compound can be dispersed in the host material at a high density, and high EO characteristics can be achieved.

Examples of the reactive functional group include a haloalkyl group, an acyl halide group, an alkoxycarbonyl group, an aryloxycarbonyl group, a hydroxy group, an amino group, an isocyanate group, an epoxy group, and a carboxy group. The reactive functional group can form a covalent bond by reacting with, for example, a hydroxy group, an amino group, or an alkoxycarbonyl group in the EO compound.

The EO film of the present embodiment can be formed using the above EO composition. The EO film can be obtained, for example, by a method including a step of applying an organic solvent solution of the EO composition onto a substrate by spin coating, and a step of heating and drying the obtained coating film.

The EO film may have a thickness of, for example, 0.01 to 100 μm.

The EO element of the present embodiment includes the above EO film. As described above, since the EO compound has excellent heat resistance while having a high hyperpolarizability, the EO element of the present embodiment has excellent EO characteristics and excellent durability with which the EO element can withstand long-term use.

An application of the EO element of the present embodiment is not limited to an optical modulator as long as the EO element has the above EO film. The EO element of the present embodiment can be used for, in addition to the optical modulator (application for ultra-high-speed, application for optical interconnect, application for optical signal processing, and the like), for example, an optical switch, an optical memory, a wavelength converter, an electric field sensor using a microwave, a millimeter wave, a terahertz wave, or the like, a biological potential sensor using a myoelectric potential, an electroencephalogram, or the like, an optical spatial modulator, an optical scanner, and the like, and can be further used for signal transmission by light between electronic circuits by combination with an electronic circuit, and the like.

### EXAMPLES

Hereinafter, the present invention will be described in more detail with reference to Examples, but the present invention is not limited to these Examples.

1. Synthesis of Compound

### (Example 1-1) Synthesis of Compound (1)

In order to synthesize a compound (1), a compound (1-a) was formylated to synthesize a compound (1-b) having a formyl group. Subsequently, a compound (1-c) was synthesized from the compound (1-b) by Suzuki coupling, and the compound (1) was synthesized from the compound (1-c) by aldol condensation.

Synthesis of Compound (1-b)

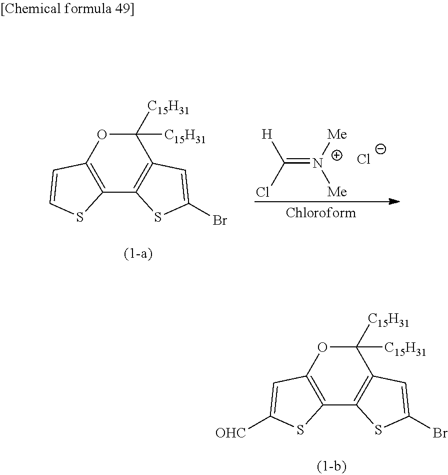

The compound (1-b) was synthesized by formylation of the compound (1-a). The compound (1-a) was synthesized according to description of WO-A-2011/136311. The inside of a 200 mL eggplant flask equipped with a Dimroth with a three-way cock at a top and containing a stirrer was purged with nitrogen, 1.00 g (1.44 mmol) of the compound (1-a) and 50 mL of dehydrated chloroform (manufactured by KANTO CHEMICAL CO., INC.) were put into the flask, and the resulting mixture was stirred with a magnetic stirrer to adjust a uniform solution. A solution was prepared by dissolving 0.45 g (3.52 mmol) of N,N-dimethylchloroiminium chloride (manufactured by Tokyo Chemical Industry Co., Ltd.) in 20 mL of dehydrated chloroform. The solution was put into the flask, and the resulting mixture was caused to react by being stirred at room temperature (25° C.) for 7.5 hours. After completion of the reaction, the reaction mixture was transferred to a separating funnel, and the organic layer was washed with 50 mL of deionized water three times. The organic layer was separated and dried over anhydrous magnesium sulfate. The insoluble matter was filtered. Then, the filtrate was concentrated and dried by a rotary evaporator to obtain the compound (1-b) as a yellow solid. The obtained amount was 1.19 g (yield: 104%).

Synthesis of Compound (1-c)

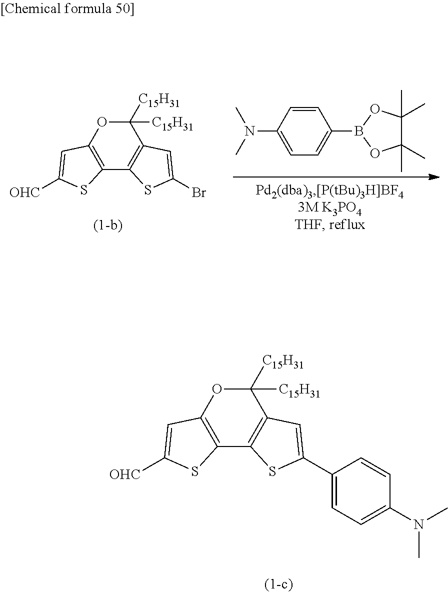

The compound (1-c) was synthesized from the compound (1-b) by Suzuki coupling. Into a 300 mL four-necked flask equipped with a Dimroth with a three-way cock at a top, a gas introduction tube, and an induction stirring type stirrer, 1.19 g (1.65 mmol) of the synthesized compound (1-b), 0.61 g (2.47 mmol) of N,N-dimethyl-4-(4,4,5,5-tetramethyl-1,3,2-dioxaborolan-2-yl) aniline (manufactured by Tokyo Chemical Industry Co., Ltd.), and 50 mL of tetrahydrofuran (manufactured by Tokyo Chemical Industry Co., Ltd.) were put, and the resulting mixture was stirred with a mechanical stirrer to adjust a uniform solution. Nitrogen gas was introduced from the three-way cock at the top, and argon gas was bubbled into the reaction mixture from the gas introduction tube to replace the inside with the inert gas. Into the flask, 0.05 g (0.055 mmol) of tris(dibenzylideneacetone) dipalladium (0) (manufactured by STREM chemical), 0.06 g (0.22 mmol) of tri-tert-butylphosphonium tetrafluoroborate (manufactured by FUJIFILM Wako Pure Chemical Corporation), and 12.36 mL of a 3 M potassium phosphate aqueous solution purged with nitrogen gas were put, and the resulting mixture was heated and stirred in an oil bath at 80° C. for seven hours to be caused to react. After completion of the reaction, the reaction mixture was transferred to a separating funnel, 100 mL of chloroform was added thereto, and the organic layer was washed with 50 mL of deionized water three times. The organic layer was separated and dried over anhydrous magnesium sulfate. The insoluble matter was filtered. Then, the filtrate was concentrated and dried by a rotary evaporator to obtain the compound (1-c) as a yellow solid. The obtained amount was 1.33 g (yield: 106%).

Synthesis of Compound (1)

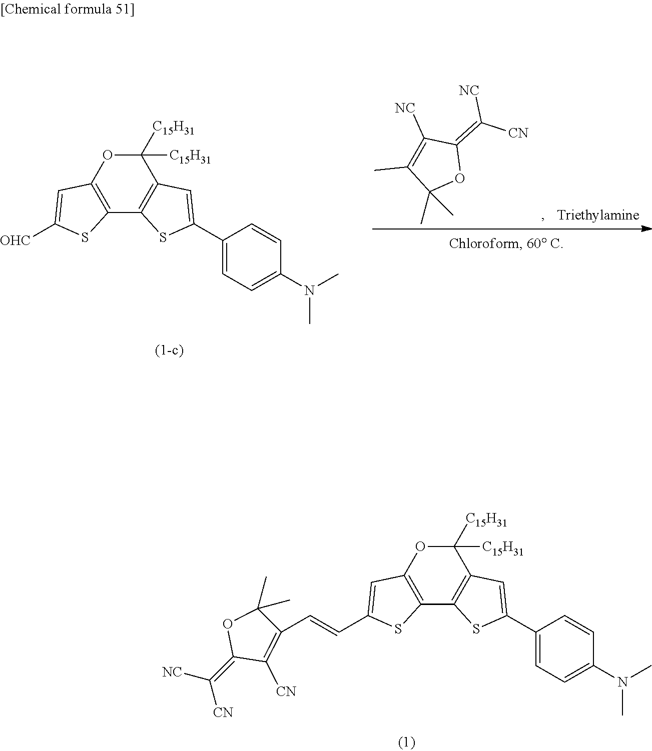

The compound (1) was synthesized from the compound (1-c) by aldol condensation. Into a 100 mL eggplant flask equipped with a Dimroth with a three-way cock at a top, 1.33 g (1.74 mmol) of the synthesized compound (1-c), 1.04 g (5.22 mmol) of 2-(3-cyano-4,5,5-trimethyl-2(5H)-furanylidene)-propanedinitrile synthesized by a method described in Chem. Mater. 2002, 14, 2393-2400, 7.05 g (69.8 mmol) of triethylamine (manufactured by Junsei Chemical Co., Ltd.), and 50 mL of dehydrated chloroform were put. A stirrer was further put thereinto, and the inside was replaced with nitrogen. The flask was immersed in an oil bath at 60° C., and reaction was caused for 16 hours while the reaction mixture was stirred with a magnetic stirrer. After completion of the reaction, the reaction mixture was transferred to a separating funnel, and the organic layer was washed with 50 mL of deionized water three times. The organic layer was separated and dried over anhydrous magnesium sulfate. The insoluble matter was filtered. Then, the filtrate was concentrated and dried by a rotary evaporator to obtain a crude product. The obtained crude product was purified with a silica gel column (hexane/ethyl acetate=8/2) to obtain the compound (1) as a blue solid. The obtained amount was 0.13 g (yield: 7.9%). The compound (1) has a divalent polycyclic condensed ring group having two or more thiophene rings and having an sp3 carbon atom as a constituent element, and further has a group represented by formula (b1). Measurement results of an 1H-NMR spectrum and a UV visible light spectrum of the compound (1) are as follows.

¹H-NMR (400 MHz, CDCl₃): δ(ppm)=7.69 (d, 1H), 7.47 (d, 2H), 7.01 (s, 1H), 6.81 (s, 1H), 6.72 (d, 2H), 6.54 (d, 1H), 3.03 (s, 6H), 1.47-1.20 (m, 56H), 0.87 (t, 6H).

UV visible light spectrum: λmax=736 nm (in chloroform)

### (Example 1-2) Synthesis of Compound (2)

In order to synthesize a compound (2), a compound (2-a) was formylated to synthesize a compound (2-b) having a formyl group. The compound (2-b) was brominated to synthesize a compound (2-c) having a bromo group. Subsequently, a compound (2-d) was synthesized from the compound (2-c) by Suzuki coupling, and the compound (2) was synthesized from the compound (2-d) by aldol condensation.

Synthesis of Compound (2-b)

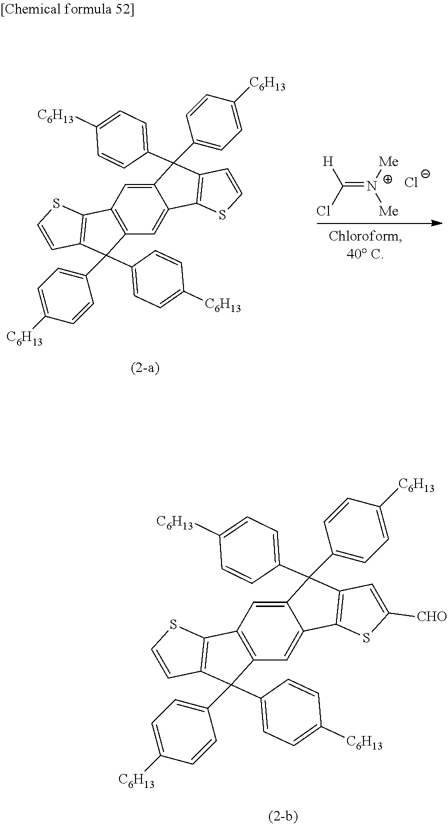

The compound (2-b) was synthesized by formylation of the compound (2-a). The compound (2-a) was synthesized by a method described in Chem. Mater. 2011, 23, 2289-2291. The inside of a 200 mL four-necked flask equipped with a Dimroth and containing a stirrer was purged with nitrogen, 2.45 g (2.7 mmol) of the compound (2-a) and 80 mL of dehydrated chloroform (manufactured by KANTO CHEMICAL CO., INC.) were put into the flask, and the resulting mixture was stirred with a magnetic stirrer to adjust a uniform solution. A solution was prepared by dissolving 0.44 g (3.4 mmol) of N,N-dimethylchloroiminium chloride (manufactured by Tokyo Chemical Industry Co., Ltd.) in 20 mL of dehydrated chloroform. The solution was put into the flask and was caused to react by being heated and stirred in an oil bath at 40° C. for four hours. After completion of the reaction, the reaction mixture was transferred to a separating funnel, and the organic layer was washed with 30 mL of deionized water three times. The organic layer was separated and dried over anhydrous magnesium sulfate. The insoluble matter was separated by filtration, and then the solvent was removed from the filtrate using a rotary evaporator. The obtained solid was washed with methanol three times, and then dried to obtain the compound (2-b) as a yellow solid. The obtained amount was 1.50 g (yield: 59%).

Synthesis of Compound (2-c)

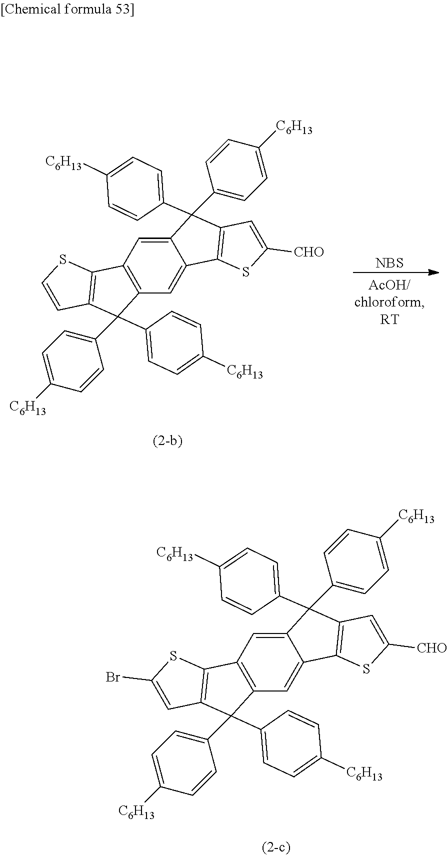

The compound (2-c) was synthesized from the compound (2-b) by bromination. Into a 200 mL three-necked flask equipped with a Dimroth with a three-way cock at a top, 1.5 g (1.6 mmol) of the synthesized compound (2-b), 10 mL of acetic acid (manufactured by Tokyo Chemical Industry Co., Ltd.), and 40 mL of dehydrated chloroform were put. A stirrer was further put thereinto, and the inside was purged with nitrogen. Thereafter, the resulting mixture was stirred with a magnetic stirrer to adjust a uniform solution. To the obtained reaction solution, 0.43 g (2.4 mmol) of N-bromosuccinimide was added. The resulting mixture was stirred at room temperature (25° C.) for five hours to be caused to react. After completion of the reaction, the reaction mixture was transferred to a separating funnel, and the organic layer was washed with 50 mL of deionized water three times. The organic layer was separated and dried over anhydrous magnesium sulfate. The insoluble matter was filtered. Then, the filtrate was concentrated and dried by a rotary evaporator. The obtained solid was washed with methanol, and then dried to obtain the compound (2-c) as a yellow solid. The obtained amount was 1.37 g (yield: 84%).

Synthesis of Compound (2-d)

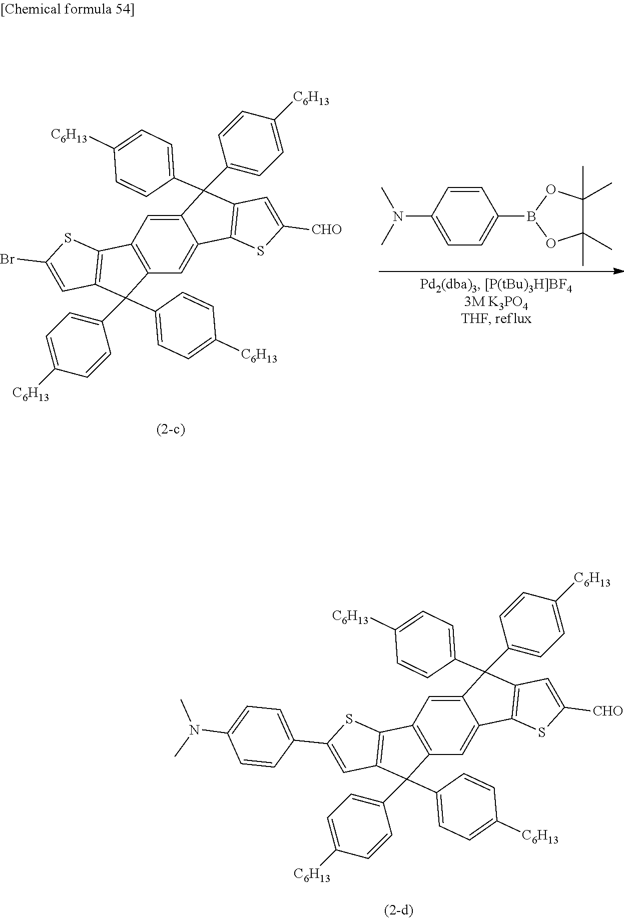

The compound (2-d) was synthesized from the compound (2-c) by Suzuki coupling. Into a 300 mL four-necked flask equipped with a Dimroth with a three-way cock at a top, a gas introduction tube, and an induction stirring type stirrer, 1.37 g (1.35 mmol) of the synthesized compound (2-c), 0.40 g (1.62 mmol) of N,N-dimethyl-4-(4,4,5,5-tetramethyl-1,3,2-dioxaborolan-2-yl) aniline (manufactured by Tokyo Chemical Industry Co., Ltd.), and 100 mL of tetrahydrofuran (manufactured by Tokyo Chemical Industry Co., Ltd.) were put, and the resulting mixture was stirred with a mechanical stirrer to adjust a uniform solution. Nitrogen gas was introduced from the three-way cock at the top, and argon gas was bubbled into the reaction mixture from the gas introduction tube to replace the inside with the inert gas. Into the flask, 0.03 g (0.027 mmol) of tris(dibenzylideneacetone) dipalladium (0) (manufactured by STREM chemical), 0.04 g (0.108 mmol) of tri-tert-butylphosphonium tetrafluoroborate (manufactured by FUJIFILM Wako Pure Chemical Corporation), and 8.10 mL of a 3 M potassium phosphate aqueous solution purged with nitrogen gas were put, and the resulting mixture was heated and stirred in an oil bath at 80° C. for nine hours to be caused to react. After completion of the reaction, the reaction mixture was transferred to a separating funnel, 100 mL of chloroform was added thereto, and the organic layer was washed with 50 mL of deionized water three times. The organic layer was separated and dried over anhydrous magnesium sulfate. The insoluble matter was filtered. Then, the filtrate was concentrated and dried by a rotary evaporator. The obtained solid was washed with methanol, and then dried to obtain the compound (2-d) as a yellow solid. The obtained amount was 1.21 g (yield: 85%).

Synthesis of Compound (2)

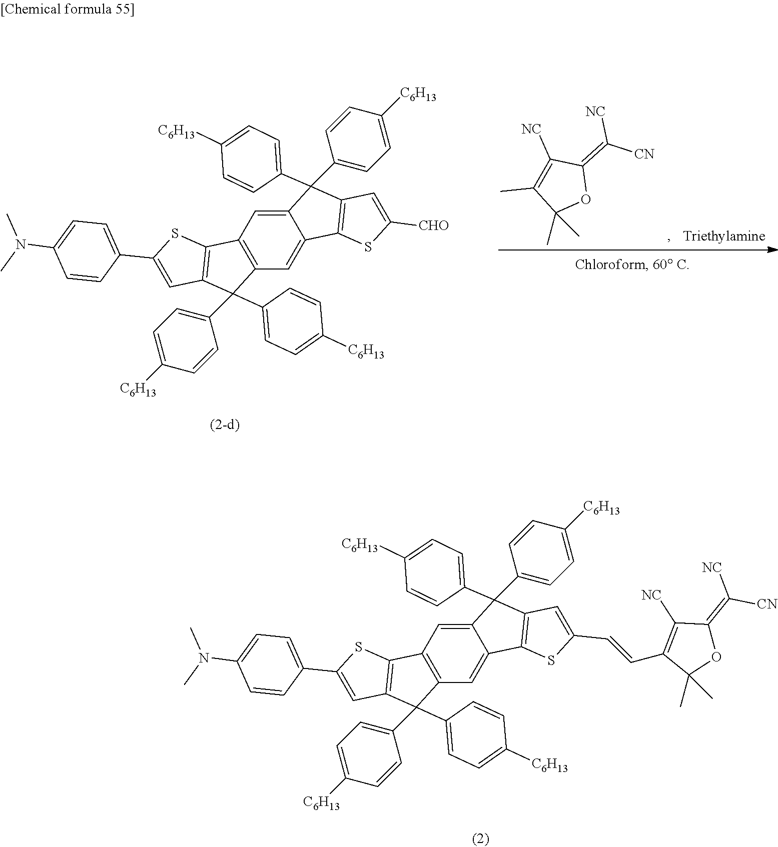

The compound (2) was synthesized from the compound (2-d) by aldol condensation. Into a 100 mL eggplant flask equipped with a Dimroth with a three-way cock at a top, 0.5 g (0.47 mmol) of the synthesized compound (2-d), 0.11 g (0.55 mmol) of 2-(3-cyano-4,5,5-trimethyl-2(5H)-furanylidene)-propanedinitrile synthesized by a method described in Chem. Mater. 2002, 14, 2393-2400, 1.92 g (19.0 mmol) of triethylamine (manufactured by Junsei Chemical Co., Ltd.), and 50 mL of dehydrated chloroform were put. A stirrer was further put thereinto, and the inside was replaced with nitrogen. The flask was immersed in an oil bath at 60° C., and reaction was caused for nine hours while the reaction mixture was stirred with a magnetic stirrer. After completion of the reaction, the reaction mixture was concentrated and dried by a rotary evaporator. The obtained solid was redissolved in methanol, and the insoluble matter was separated by filtration. Thereafter, the filtrate was concentrated and dried by a rotary evaporator to obtain a crude product as a blue solid. The obtained crude product was purified with a silica gel column (toluene/methanol=97.5/2.5) to obtain the compound (2) as a blue solid. The obtained amount was 0.05 g (yield: 8.5%). The compound (2) has a divalent polycyclic condensed ring group having two or more thiophene rings and having an sp3 carbon atom as a constituent element, and further has a group represented by formula (b1). Measurement results of an ¹H-NMR spectrum and a UV visible light spectrum of the compound (2) are as follows.

¹H-NMR (400 MHz, CDCl₃): δ(ppm)=7.79 (d, 1H), 7.6-6.8 (m, 22H), 6.68 (d, 2H), 6.57 (d, 1H), 2.96 (s, 6H), 2.6-2.4 (m, 8H), 1.7-1.5 (m, 8H), 1.4-1.2 (m, 24H), 0.86 (t, 12H).

UV visible light spectrum: λmax=656 nm (in chloroform)

### (Example 1-3) Synthesis of Compound (3)

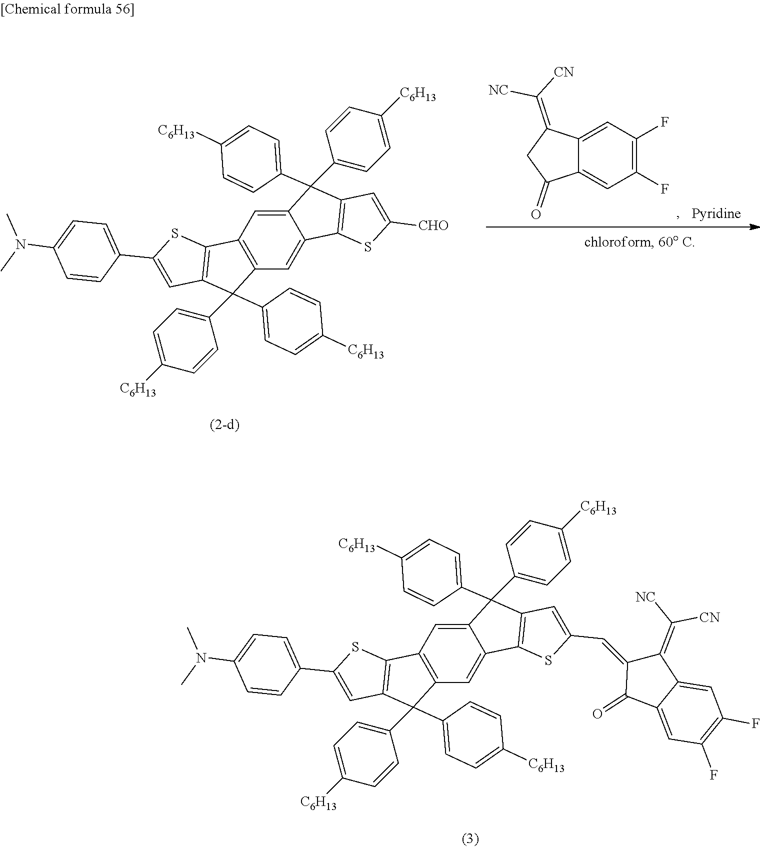

A compound (3) was synthesized from the compound (2-d) by aldol condensation. Into a 100 mL eggplant flask equipped with a Dimroth with a three-way cock at a top, 0.7 g (0.66 mmol) of the synthesized compound (2-d), 0.18 g (0.78 mmol) of 2-(5,6-difluoro-3-oxo-2,3-dihydro-1H-inden-1-ylidene)malononitrile synthesized by a method described in J. Am. Chem. Soc. 2017, 139, 1336-1343, 0.26 g (3.29 mmol) of pyridine (manufactured by Junsei Chemical Co., Ltd.), and 40 mL of dehydrated chloroform were put. A stirrer was further put thereinto, and the inside was replaced with nitrogen. Reaction was caused at room temperature (25° C.) for three hours while the reaction mixture was stirred with a magnetic stirrer. After completion of the reaction, the reaction mixture was concentrated by a rotary evaporator. The obtained viscous liquid was redissolved in methanol, and the insoluble matter was separated by filtration. Thereafter, the filtrate was concentrated by a rotary evaporator. The obtained viscous liquid was washed with hexane and filtered to obtain a crude product as a blue solid. The obtained crude product was purified with a silica gel column (chloroform/methanol=1/2) to obtain the compound (3) as a blue solid. The obtained amount was 0.47 g (yield: 56%). The compound (3) has a divalent polycyclic condensed ring group having two or more thiophene rings and having an sp3 carbon atom as a constituent element, and further has a group represented by formula (a2). Measurement results of an ¹H-NMR spectrum and a UV visible light spectrum of the compound (3) are as follows.

¹H-NMR (400 MHz, CDCl₃): δ(ppm)=8.84 (s, 1H), 7.6-6.8 (m, 24H), 6.68 (d, 2H), 2.98 (s, 3H), 2.6-2.4 (m, 8H), 1.7-1.5 (m, 8H), 1.5-1.1 (m, 24H), 0.84 (t, 12H).

UV visible light spectrum: λmax=649 nm (in chloroform)

### (Example 1-4) Synthesis of Compound (4)

In order to synthesize a compound (4), a compound (4-a) was formylated to synthesize a compound (4-b) having a formyl group, and the compound (4-b) was brominated to synthesize a compound (4-c) having a bromo group. Subsequently, a compound (4-d) was synthesized from the compound (4-c) by Suzuki coupling, and the compound (4) was synthesized from the compound (4-d) by aldol condensation.

Synthesis of Compound (4-b)

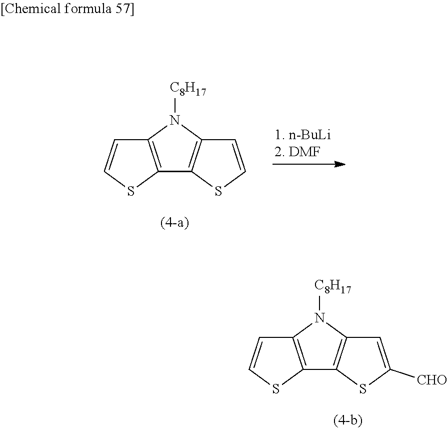

The compound (4-b) was synthesized by formylation of the compound (4-a). Into a 200 mL eggplant flask equipped with a Dimroth with a three-way cock and a gas introduction tube, 2.47 g (8.47 mmol) of 4-n-octyl-4H-dithieno [3,2-b:2′,3′-d] pyrrole (compound (4-a), manufactured by Tokyo Chemical Industry Co., Ltd.) and 49 mL of dehydrated tetrahydrofuran (manufactured by FUJIFILM Wako Pure Chemical Corporation) were put. A stirrer was further put thereinto, the inside was replaced with nitrogen, and the solution was cooled to −60° C. 5.90 mL (9.32 mmol) of a 1.6 M n-butyl lithium tetrahydrofuran solution (manufactured by KANTO CHEMICAL CO., INC.) was added dropwise thereto, and then the resulting mixture was stirred at −60° C. for 30 minutes to be caused to react. 1.24 g (16.95 mmol) of dehydrated N,N-dimethylformamide (manufactured by FUJIFILM Wako Pure Chemical Corporation) was added thereto, and the temperature was raised to 0° C. over one hour. After completion of the reaction, 2.5 mL of methanol was added thereto for quenching, the insoluble matter was separated by filtration, and the filtrate was concentrated by a rotary evaporator to obtain the compound (4-b) as an oil. The obtained amount was 2.08 g (yield: 77%). A measurement result of an ¹H-NMR spectrum of the compound (4-b) is as follows.

¹H-NMR (400 MHz, CDCl₃): δ(ppm)=9.85 (s, 1H), 7.63 (s, 1H), 7.36 (d, 1H), 7.00 (d, 1H), 4.20 (t, 2H), 1.90-1.81 (m, 2H), 1.35-1.19 (m, 10H), 0.85 (t, 3H).

Synthesis of Compound (4-c)

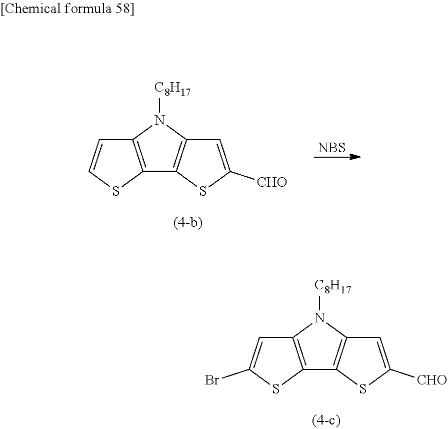

The compound (4-c) was synthesized from the compound (4-b) by bromination. Into a 100 mL eggplant flask equipped with a gas introduction tube, 0.500 g (1.57 mmol) of the compound (4-b) and 5.0 mL of dehydrated tetrahydrofuran (manufactured by FUJIFILM Wako Pure Chemical Corporation) were put. A stirrer was further put thereinto, the inside was replaced with nitrogen, and the solution was cooled to −40° C. 0.293 g (1.65 mmol) of N-bromosuccinimide (manufactured by Tokyo Chemical Industry Co., Ltd.) was added thereto, and then the resulting mixture was stirred at −40° C. for one hour to be caused to react. The temperature of the reaction mixture was raised to room temperature (25° C.), then 5 g of a 10% sodium sulfite aqueous solution and 9.3 mL of toluene were added thereto, and the organic layer was washed with deionized water. After washing, the organic layer was dried over anhydrous magnesium sulfate. Thereafter, the insoluble matter was separated by filtration, and the filtrate was concentrated and dried by a rotary evaporator to obtain the compound (4-c). The obtained amount was 0.535 g (yield: 86%).

Synthesis of Compound (4A-b)

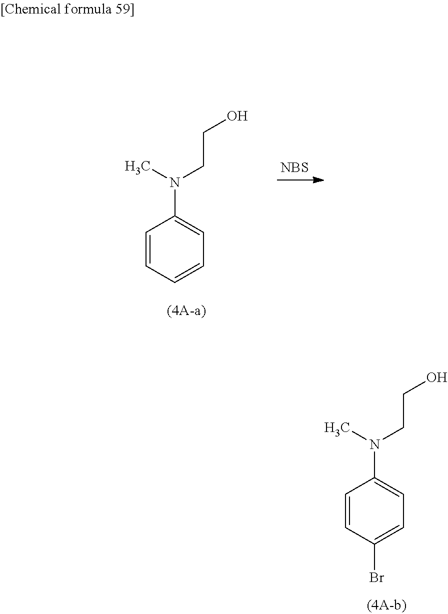

Into a 1 L eggplant flask equipped with a gas introduction tube and an induction stirring type stirrer, 55.47 g (366.8 mmol) of 2-(methylphenylamino) ethanol (compound (4A-a), manufactured by Tokyo Chemical Industry Co., Ltd.) and 832 mL of dehydrated dimethylformamide (manufactured by FUJIFILM Wako Pure Chemical Corporation) were put. The inside was replaced with nitrogen, and the solution was cooled to −40° C. 65.29 g (366.8 mmol) of N-bromosuccinimide (manufactured by Tokyo Chemical Industry Co., Ltd.) was added thereto. Thereafter, the temperature was raised to −15° C., and reaction was caused for two hours. The temperature of the reaction mixture was raised to room temperature (25° C.), then 455 g of a 10% sodium sulfite aqueous solution and 1110 mL of toluene were added thereto, and the organic layer was washed with deionized water. After washing, the organic layer was dried over anhydrous magnesium sulfate. Thereafter, the insoluble matter was separated by filtration, and the filtrate was concentrated and dried by a rotary evaporator to obtain a compound (4A-b) as a colorless oil. The obtained amount was 72.05 g (yield: 85%). A measurement result of an ¹H-NMR spectrum of the compound (4A-b) is as follows.

¹H-NMR (400 MHz, CDCl₃): δ(ppm)=7.27 (d, 2H), 6.63 (d, 2H), 3.80-3.74 (m, 2H), 3.42 (t, 2H), 2.92 (3H), 1.90 (t, 1H).

Synthesis of Compound (4A-c)

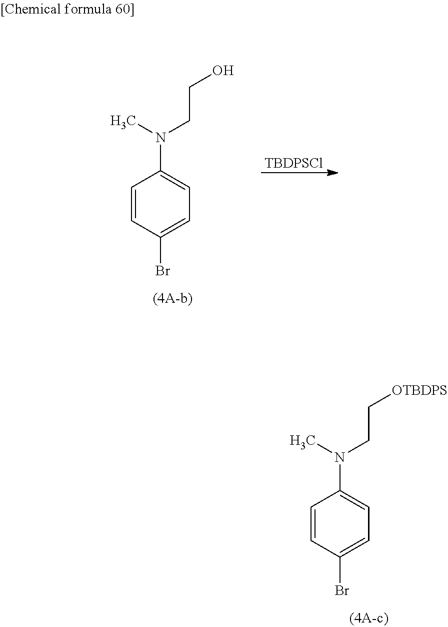

Into a 1 L eggplant flask equipped with a gas introduction tube and an induction stirring type stirrer, 71.93 g (312.6 mmol) of the compound (4A-b), 42.56 g (625.2 mmol) of imidazole (manufactured by Tokyo Chemical Industry Co., Ltd.), and 832 mL of dehydrated tetrahydrofuran (manufactured by FUJIFILM Wako Pure Chemical Corporation) were put. The inside was replaced with nitrogen, and the solution was cooled to 0° C. 88.51 g (322.0 mmol) of tert-butyldiphenylchlorosilane (TBDPSCl, manufactured by Tokyo Chemical Industry Co., Ltd., hereinafter, a tert-butyldiphenylsilyl group is also referred to as “TBDPS”) was added thereto. Thereafter, the temperature was raised to room temperature (25° C.), and reaction was caused for three hours. 2929 mL of hexane was added to the reaction mixture, and the organic layer was washed with deionized water. After washing, the organic layer was dried over anhydrous magnesium sulfate. Thereafter, the insoluble matter was separated by filtration, and the filtrate was concentrated and dried by a rotary evaporator to obtain a compound (4A-c) as a colorless oil. The obtained amount was 145.5 g (yield: 99%).

Synthesis of Compound (4A-d)

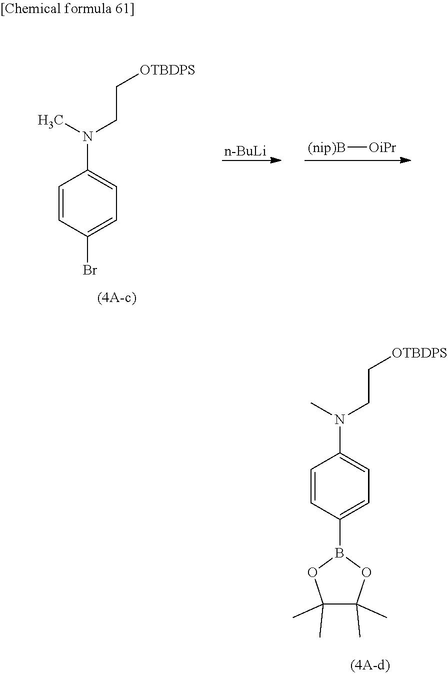

Into a 3 L eggplant flask equipped with a three-way cock at a top and a gas introduction tube, 145.6 g (310.8 mmol) of the compound (4A-c) and 1456 mL of dehydrated tetrahydrofuran (manufactured by FUJIFILM Wako Pure Chemical Corporation) were put. The inside was replaced with nitrogen, and the solution was cooled to −65° C. 239 mL (372.9 mmol) of a 1.6 M n-butyl lithium tetrahydrofuran solution (manufactured by KANTO CHEMICAL CO., INC.) was added dropwise thereto, and then the resulting mixture was stirred for one hour to be caused to react. 75.17 g (404.0 mmol) of 2-isopropoxy-4,4,5,5-tetramethyl-1,3,2-dioxaborolane (manufactured by Tokyo Chemical Industry Co., Ltd.) was added thereto, and the temperature was raised to room temperature (25° C.) over two hours. 2884 mL of hexane was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The crude product was crystallized twice using acetonitrile, and then the solid was dried under reduced pressure to obtain a compound (4A-d) as a white solid. The obtained amount was 124.1 g (yield: 77%). A measurement result of an ¹H-NMR spectrum of the compound (4A-d) is as follows.

¹H-NMR (400 MHz, CDCl₃): δ(ppm)=7.64-7.57 (m, 6H), 7.42-7.31 (m, 6H), 6.54 (d, 2H), 3.79 (t, 2H), 3.49 (t, 2H), 2.95 (s, 3H), 1.31 (s, 12H), 1.02 (s, 9H).

Synthesis of Compound (4-d)

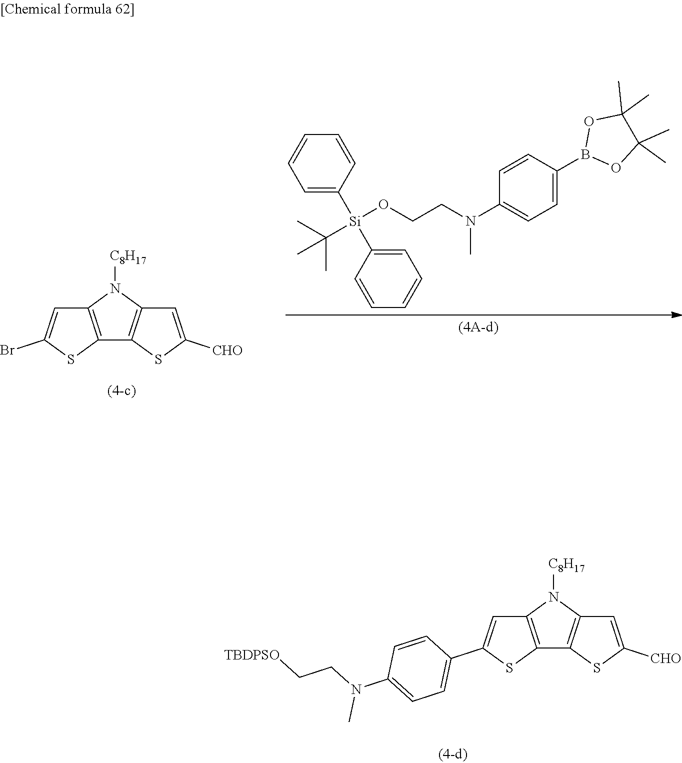

The compound (4-d) was synthesized from the compound (4-c) by Suzuki coupling. Into a 20 mL four-necked flask equipped with a Dimroth with a three-way cock at a top and a gas introduction tube, 0.54 g (1.34 mmol) of the synthesized compound (4-c), 0.83 g (1.61 mmol) of the synthesized compound (4A-d), and 8 mL of dehydrated tetrahydrofuran (manufactured by FUJIFILM Wako Pure Chemical Corporation) were put. A stirrer was further put thereinto, and the inside was replaced with nitrogen. Into the flask, 0.04 g (0.034 mmol) of tris(dibenzylideneacetone) dipalladium (0) (manufactured by N.E. CHEMCAT CORPORATION), 0.04 g (0.13 mmol) of tri-tert-butylphosphonium tetrafluoroborate (manufactured by FUJIFILM Wako Pure Chemical Corporation), and 1.79 mL of a 3 M potassium phosphate aqueous solution purged with nitrogen gas were put, and the resulting mixture was heated and stirred in an oil bath at 50° C. for two hours to be caused to react. 10 mL of toluene was added to the reaction mixture, and the organic layer was washed with deionized water. The organic layer was dried over anhydrous magnesium sulfate, and the insoluble matter was filtered. Thereafter, the filtrate was concentrated by a rotary evaporator to obtain a crude product. Methyl-tert-butyl ether was added to the obtained crude product. The precipitate was separated by filtration, further washed with methyl-tert-butyl ether, and then dried under reduced pressure to obtain the compound (4-d) as a pink solid. The obtained amount was 0.45 g (yield: 48%). A measurement result of an ¹H-NMR spectrum of the compound (4-d) is as follows.

¹H-NMR (400 MHz, CDCl₃): δ(ppm)=9.83 (s, 1H), 7.64-7.61 (m, 4H), 7.59 (s, 1H) 7.48-7.38 (m, 4H), 7.38 (m, 4H), 7.01 (s, 1H), 6.58 (d, 2H), 4.20 (t, 2H), 3.82 (t, 2H), 3.52 (t, 2H), 2.99 (s, 3H), 1.91-1.86 (m, 2H), 1.37-1.19 (m, 10H), 1.03 (s, 9H), 0.85 (t, 3H).

Synthesis of Compound (4)

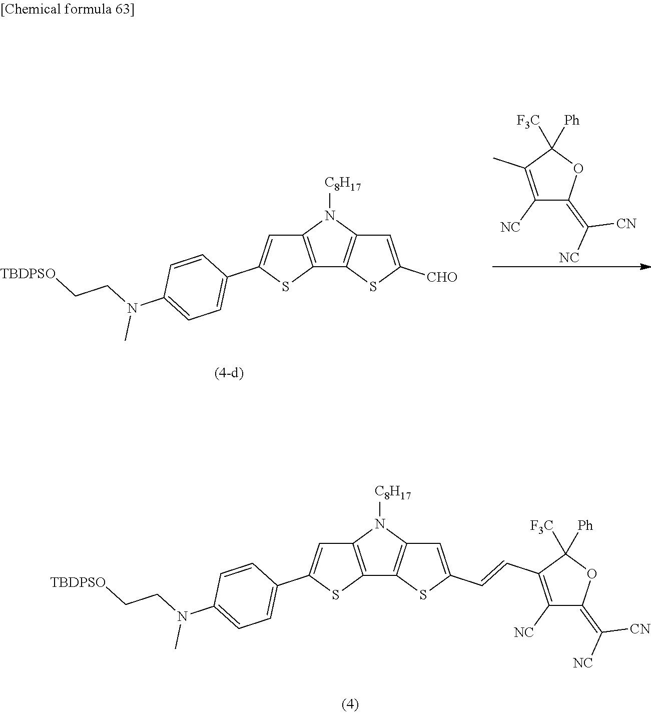

The compound (4) was synthesized from the compound (4-d) by aldol condensation. Into a 20 mL eggplant flask equipped with a three-way cock and a gas introduction tube, 0.45 g (0.63 mmol) of the synthesized compound (4-d), 0.28 g (0.88 mmol) of 2-(3-cyano-4-methyl-5-phenyl-5-(trifluoromethyl)-2(5H)-furanylidene)-propanedinitrile (manufactured by iChemical), 5 mL of dehydrated ethanol, and 5 mL of dehydrated chloroform were put. A stirrer was further put thereinto, and the inside was replaced with nitrogen. Reaction was caused at room temperature (25° C.) for 18 hours while the reaction mixture was stirred with a magnetic stirrer. After completion of the reaction, the reaction mixture was concentrated by a rotary evaporator. Methyl-tert-butyl ether was added to the concentrate. The precipitate was separated by filtration, and further washed with methyl-tert-butyl ether to obtain the compound (4) as a green solid. The obtained amount was 0.61 g (yield: 96%). The compound (4) has a divalent polycyclic condensed ring group having two or more thiophene rings and having a nitrogen atom as a constituent element, and further has a group represented by formula (b1). Measurement results of an ¹H-NMR spectrum and a UV visible light spectrum of the compound (4) are as follows.

¹H-NMR (400 MHz, CD₂Cl₂): δ(ppm)=8.03 (m, 1H), 7.60 (d, 4H), 7.55-7.52 (m, 5H), 7.47 (d, 2H), 7.42-7.36 (m, 2H), 7.35-7.30 (m, 4H), 7.28 (brs, 1H), 7.01 (s, 1H), 6.62 (d, 2H), 6.48 (d, 1H), 4.11 (t, 2H), 3.81 (t, 2H), 3.56 (t, 2H), 3.01 (s, 3H), 1.86-1.80 (m, 2H), 1.32-1.19 (m, 10H), 1.00 (s, 9H), 0.83 (t, 3H).

UV visible light spectrum: λmax=794 nm (in chloroform)

### (Example 1-5) Synthesis of Compound (5)

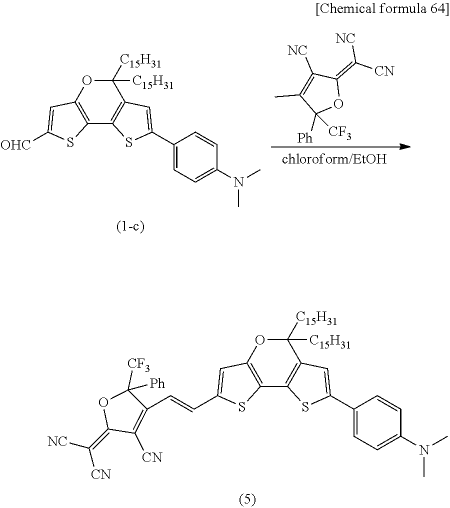

A compound (5) was synthesized from the compound (1-c) by aldol condensation. Into a 100 mL eggplant flask equipped with a Dimroth with a three-way cock at a top, 0.50 g (0.66 mmol) of the synthesized compound (1-c), 0.31 g (0.98 mmol) of 2-(3-cyano-4-methyl-5-phenyl-5-(trifluoromethyl)-2(5H)-furanylidene)-propanedinitrile (manufactured by iChemical), 15 mL of dehydrated ethanol, and 1.5 mL of dehydrated chloroform were put. A stirrer was further put thereinto, and the inside was replaced with nitrogen. Reaction was caused at room temperature (25° C.) for eight hours while the reaction mixture was stirred with a magnetic stirrer. After completion of the reaction, the reaction mixture was concentrated and dried by a rotary evaporator. The obtained solid was washed with hexane, and further washed with methanol to obtain a crude product. The obtained crude product was purified with a silica gel column (hexane/ethyl acetate=6/4) to obtain the compound (5) as a blue solid. The obtained amount was 0.63 g (yield: 91%). The compound (5) has a divalent polycyclic condensed ring group having two or more thiophene rings and having an sp3 carbon atom as a constituent element, and further has a group represented by formula (b1). Measurement results of an ¹H-NMR spectrum and a UV visible light spectrum of the compound (5) are as follows.

¹H-NMR (400 MHz, CDCl₃): δ(ppm)=7.69 (d, 1H), 7.54-7.46 (m, 5H), 6.92 (s, 1H), 6.81 (s, 1H), 6.70 (d, 2H), 6.57 (d, 1H), 3.03 (s, 6H), 1.47-1.20 (m, 56H), 0.87 (t, 6H).

UV visible light spectrum: λmax=824 nm (in chloroform)

### (Example 1-6) Synthesis of Compound (6)

In order to synthesize a compound (6), a compound (6-a) was alkylated to synthesize a compound (6-b). Subsequently, the compound (6-b) was brominated and formylated to synthesize a compound (6-c). Subsequently, a compound (6-d) was synthesized from the compound (6-c) by Suzuki coupling, and the compound (6) was synthesized from the compound (6-d) by aldol condensation.

Synthesis of Compound (6-b)

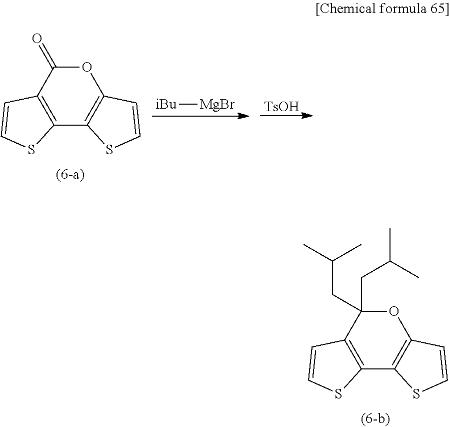

The compound (6-b) was synthesized by alkylation of the compound (6-a). The compound (6-a) was synthesized by a method described in WO-A-2011/052709. Into a 3 L eggplant flask equipped with a three-way cock and a gas introduction tube, 100 g (480 mmol) of the compound (6-a) and 800 mL of dehydrated tetrahydrofuran (manufactured by KANTO CHEMICAL CO., INC.) were put. A stirrer was further put thereinto, the inside was replaced with nitrogen, and the solution was cooled to 0° C. 1056 mL (1056 mmol) of a 1 M isobutylmagnesium bromide tetrahydrofuran solution (manufactured by Tokyo Chemical Industry Co., Ltd.) was added dropwise thereto, and then the resulting mixture was stirred for 24 hours to be caused to react. After completion of the reaction, 1000 mL of deionized water and 2500 mL of toluene were added thereto, and the organic layer was washed with deionized water. After washing, the organic layer was dried over anhydrous magnesium sulfate. Thereafter, the insoluble matter was separated by filtration, and the filtrate was concentrated by a rotary evaporator to obtain a reaction product as a brown oily matter. The obtained amount was 144 g.

Subsequently, into a 3 L eggplant flask equipped with a three-way cock at a top and a gas introduction tube, the reaction product, 21 g (111 mmol) of p-toluenesulfonic acid monohydrate (manufactured by Tokyo Chemical Industry Co., Ltd.), and 1440 mL of dehydrated toluene (manufactured by KANTO CHEMICAL CO., INC.) were put. A stirrer was further put thereinto, and the inside was replaced with nitrogen. Reaction was caused under reflux conditions for one hour while the reaction mixture was stirred with a magnetic stirrer. The obtained reaction mixture was washed with deionized water. After washing, the organic layer was dried over anhydrous magnesium sulfate. Thereafter, the insoluble matter was separated by filtration, and the filtrate was concentrated by a rotary evaporator. The concentrate was purified with a silica gel column (hexane) to obtain the compound 6-b) as pale orange oily matter. The obtained amount was 74 g (yield: 43%). A measurement result of an ¹H-NMR spectrum of the compound (6-b) is as follows.

¹H-NMR (400 MHz, CDCl₃): δ(ppm)=7.10 (d, 1H), 6.95 (d, 1H), 6.68 (d, 1H), 6.64 (d, 1H), 1.90-1.72 (m, 6H), 0.87 (d, 6H), 0.84 (d, 6H).

Synthesis of Compound (6-c)

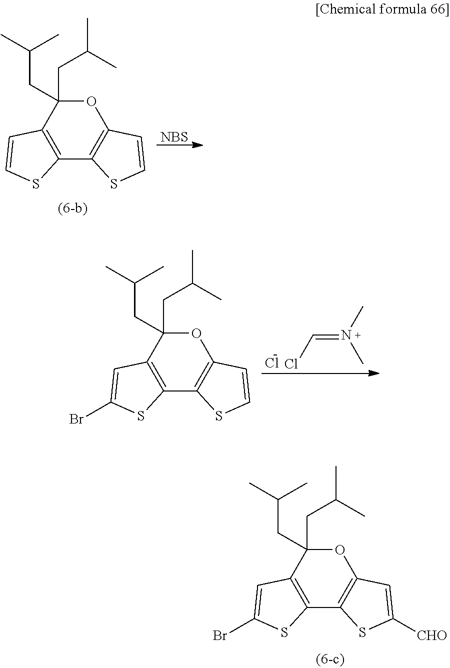

The compound (6-b) was brominated and formylated to synthesize a compound (6-c). Into a 2 L eggplant flask equipped with a three-way cock at a top and a gas introduction tube, 38.7 g (126 mmol) of the synthesized compound (6-b) and 387 mL of dehydrated tetrahydrofuran (manufactured by FUJIFILM Wako Pure Chemical Corporation) were put. A stirrer was further put thereinto, the inside was replaced with nitrogen, and the solution was cooled to −40° C. 22.7 g (128 mmol) of N-bromosuccinimide (manufactured by Tokyo Chemical Industry Co., Ltd.) was added thereto, and then the resulting mixture was stirred at −40° C. for two hours to be caused to react. The temperature of the reaction mixture was raised to room temperature (25° C.). Thereafter, 97.1 g (758 mmol) of (chloromethylene) dimethyliminium chloride (manufactured by Tokyo Chemical Industry Co., Ltd.) and 487 mL of dehydrated dimethylformamide were added thereto, and the resulting mixture was caused to react at 60° C. for five hours while being stirred with a magnetic stirrer. 731 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was purified with a silica gel column (hexane/ethyl acetate=80/20) to obtain the compound (6-c) as a green solid. The obtained amount was 36.4 g (yield: 70%). A measurement result of an ¹H-NMR spectrum of the compound (6-c) is as follows.

¹H-NMR (400 MHz, CDCl₃): δ(ppm)=9.74 (s, 1H), 7.21 (s, 1H), 6.69 (s, 1H), 1.86-1.71 (m, 6H), 0.87 (d, 6H), 0.85 (d, 6H).

Synthesis of Compound (6-d)

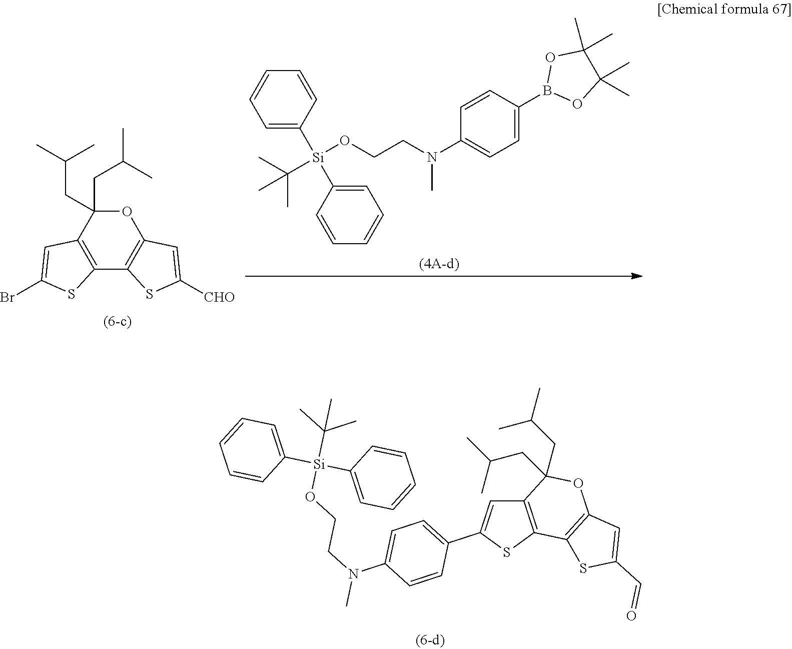 [SMILES: CC(C)CC1(CC(C)C)Oc2cc(C=O)sc2-c2sc(Br)cc21 ; CN(CCO[Si](c1ccccc1)(c1ccccc1)C(C)(C)C)c1ccc(B2OC(C)(C)C(C)(C)O2)cc1 ; CC(C)CC1(CC(C)C)Oc2cc(C=O)sc2-c2sc(-c3ccc(N(C)CCO[Si](c4ccccc4)(c4ccccc4)C(C)(C)C)cc3)cc21]

The compound (6-d) was synthesized from the compound (6-c) by Suzuki coupling. Into a 300 mL four-necked flask equipped with a Dimroth with a three-way cock at a top and a gas introduction tube, 6.00 g (14.5 mmol) of the synthesized compound (6-c), 8.23 g (16.0 mmol) of the synthesized compound (4A-d), and 90 mL of dehydrated tetrahydrofuran (manufactured by FUJIFILM Wako Pure Chemical Corporation) were put. A stirrer was further put thereinto, and the inside was replaced with nitrogen. 0.42 g (0.36 mmol) of tris(dibenzylideneacetone) dipalladium (0) (manufactured by N.E. CHEMCAT CORPORATION), 0.44 g (1.45 mmol) of tri-tert-butylphosphonium tetrafluoroborate (manufactured by FUJIFILM Wako Pure Chemical Corporation), and 19.4 mL of a 3 M potassium phosphate aqueous solution purged with nitrogen gas were put, and the resulting mixture was heated and stirred in an oil bath at 50° C. for two hours to be caused to react. 120 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was purified with a reversed-phase silica gel column (methanol/ethyl acetate=90/10 to 80/20) to obtain the compound (6-d) as a green solid. The obtained amount was 4.92 g (yield: 49%). A measurement result of an ¹H-NMR spectrum of the compound (6-d) is as follows.

¹H-NMR (400 MHz, CDCl₃): δ(ppm)=9.72 (s, 1H), 7.62 (dd, 4H), 7.46-7.31 (m, 8H), 7.22 (s, 1H), 6.73 (s, 1H), 6.56 (d, 2H), 3.81 (t, 2H), 3.51 (t, 2H), 2.98 (s, 3H), 1.92-1.76 (m, 6H), 1.03 (s, 9H), 0.89 (d, 6H), 0.86 (d, 6H).

Synthesis of Compound (6)

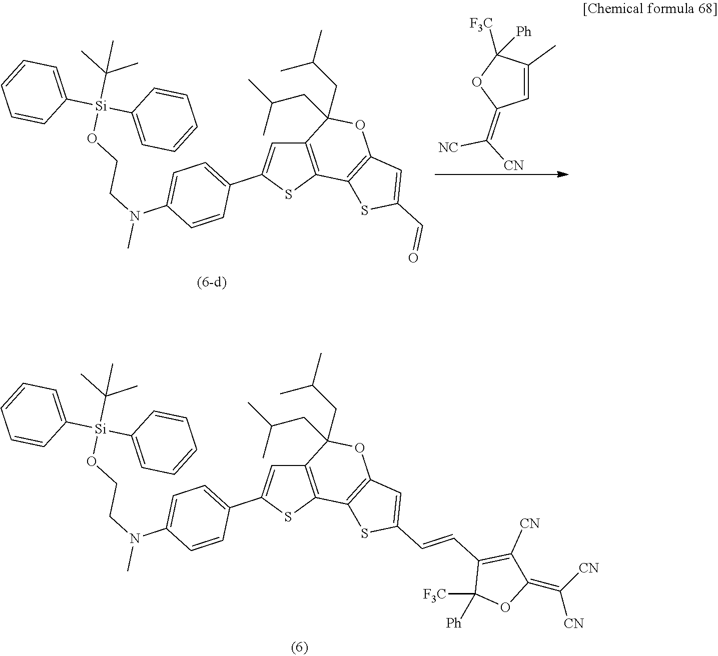 [SMILES: CC(C)CC1(CC(C)C)Oc2cc(C=O)sc2-c2sc(-c3ccc(N(C)CCO[Si](c4ccccc4)(c4ccccc4)C(C)(C)C)cc3)cc21 ; CC1=CC(=C(C#N)C#N)OC1(c1ccccc1)C(F)(F)F ; CC(C)CC1(CC(C)C)Oc2cc(/C=C/C3=C(C#N)C(=C(C#N)C#N)OC3(c3ccccc3)C(F)(F)F)sc2-c2sc(-c3ccc(N(C)CCO[Si](c4ccccc4)(c4ccccc4)C(C)(C)C)cc3)cc21]

The compound (6) was synthesized from the compound (6-d) by aldol condensation. Into a 500 mL eggplant flask equipped with a three-way cock and a gas introduction tube, 4.92 g (6.81 mmol) of the synthesized compound (6-d), 2.58 g (8.18 mmol) of 2-(3-cyano-4-methyl-5-phenyl-5-(trifluoromethyl)-2(5H)-furanylidene)-propanedinitrile (manufactured by iChemical), 64 mL of dehydrated ethanol, and 80 mL of dehydrated chloroform were put. A stirrer was further put thereinto, and the inside was replaced with nitrogen. Reaction was caused at room temperature (25° C.) for 30 hours while the reaction mixture was stirred with a magnetic stirrer. After completion of the reaction, the reaction mixture was concentrated by a rotary evaporator. Ethanol was added to the concentrate, the precipitate was separated by filtration, and the precipitate was further washed with ethanol to obtain a crude product. The crude product was purified with a reversed-phase silica gel column (acetonitrile/ethyl acetate=100/0 to 90/10) to obtain the compound (6) as a green solid. The obtained amount was 6.30 g (yield: 70%). The compound (6) has a divalent polycyclic condensed ring group having two or more thiophene rings and having an sp3 carbon atom as a constituent element, and further has a group represented by formula (b1). Measurement results of an ¹H-NMR spectrum and a UV visible light spectrum of the compound (6) are as follows.

¹H-NMR (400 MHz, CDCl₃): δ(ppm)=7.61 (dd, 4H), 7.55-7.47 (m, 5H), 7.44-7.31 (m, 9H), 6.90 (s, 1H), 6.77 (s, 1H), 6.59-6.53 (m, 3H), 3.80 (t, 2H), 3.53 (t, 2H), 3.00 (s, 3H), 1.84-1.76 (m, 6H), 1.03 (s, 9H), 0.85 (d, 6H), 0.83 (d, 6H).

UV visible light spectrum: λmax=833 nm (in chloroform)

### (Example 1-7) Synthesis of Compound (7)

In order to synthesize a compound (7), a compound (7-a) was brominated to synthesize a compound (7-b). Subsequently, the compound (7-b) was formylated to synthesize a compound (7-c). Subsequently, a compound (7-d) was synthesized from the compound (7-c) by Suzuki coupling, and the compound (7) was synthesized from the compound (7-d) by aldol condensation.

Synthesis of Compound (7-b)

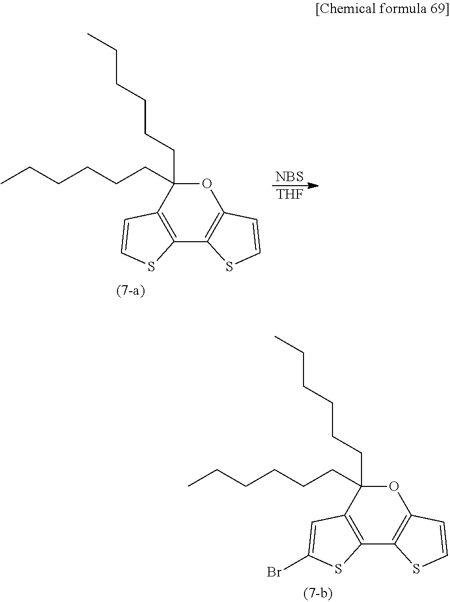 [SMILES: CCCCCCC1(CCCCCC)Oc2ccsc2-c2sccc21 ; "NBS" ; CCCCCCC1(CCCCCC)Oc2ccsc2-c2sc(Br)cc21 ; "THF"]

The compound (7-b) was synthesized by bromination of the compound (7-a). The compound (7-a) was synthesized by a method described in WO-A-2013/047858. Into a 500 mL three-necked flask equipped with a Dimroth with a three-way cock at a top and a 100 mL equilibrium type dropping funnel, 10.00 g (27.6 mmol) of the compound (7-a) and 100 mL of dehydrated tetrahydrofuran (THF, manufactured by FUJIFILM Wako Pure Chemical Corporation) were put. A stirrer was further put thereinto, and the inside was replaced with nitrogen. Thereafter, the flask containing the reaction mixture was immersed in a dry ice acetone bath, and the reaction mixture was cooled to −40° C. A solution adjusted by dissolving 5.16 g (29.0 mmol) of N-bromosuccinimide (NBS, manufactured by Tokyo Chemical Industry Co., Ltd.) in 50 mL of dehydrated THF was put into the dropping funnel. A THF solution of NBS was slowly added dropwise from the dropping funnel such that the temperature of the reaction mixture did not exceed −40° C. After completion of the dropwise addition, stirring was continued at −40° C. for three hours. Thereafter, the flask was taken out of the dry ice acetone bath and further stirred at room temperature (25° C.) for 16 hours for reaction. After completion of the reaction, the reaction mixture was transferred to a 500 mL eggplant flask and concentrated by a rotary evaporator. 200 mL of chloroform and 200 mL of deionized water were added to the obtained concentrate, the product was extracted, and the organic layer was separated. The obtained organic layer was further washed with 100 mL of deionized water three times, and the separated organic layer was dried over anhydrous magnesium sulfate. Thereafter, the insoluble matter was separated by filtration, and the filtrate was concentrated and dried by a rotary evaporator to obtain the compound (7-b). The obtained amount was 10.93 g (yield: 90%). A measurement result of an ¹H-NMR spectrum of the compound (7-b) is as follows.

¹H-NMR (400 MHz, CD₃COCD₃): δ(ppm)=7.26 (d, 1H), 7.04 (s, 1H), 6.72 (d, 1H), 1.98-1.85 (m, 4H), 1.50-1.17 (m, 16H), 0.94-0.77 (m, 6H).

Synthesis of Compound (7-c)

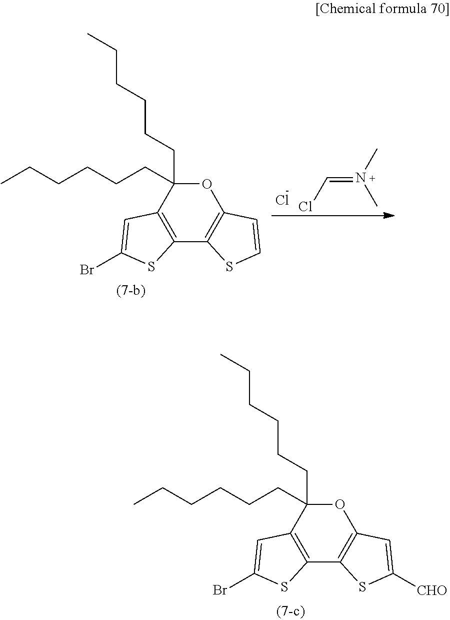

The compound (7-c) was synthesized by formylation of the compound (7-b). Into a 500 mL three-necked flask equipped with a three-way cock, 8.00 g (18.1 mmol) of the compound (7-b) and 150 mL of dehydrated chloroform (manufactured by FUJIFILM Wako Pure Chemical Corporation) were put. A stirrer was further put thereinto, and the inside was replaced with nitrogen. At room temperature (25° C.), 4.64 g (36.2 mmol) of a Vilsmeir reagent (manufactured by Tokyo Chemical Industry Co., Ltd.) was added thereto in three portions while the reaction mixture was stirred with a magnetic stirrer. After completion of the addition, the resulting mixture was further stirred for 24 hours to be caused to react. After completion of the reaction, 50 mL of deionized water was added thereto to quench the reaction. The organic layer was separated, then further washed with 50 mL of deionized water, and then dried over anhydrous magnesium sulfate. The insoluble matter was separated by filtration. The filtrate was transferred to a 500 mL eggplant flask and concentrated by a rotary evaporator. 200 mL of ethyl acetate and 200 mL of deionized water were added to the obtained concentrate, and the product was extracted. The contents of the eggplant flask were transferred to a 500 mL separating funnel to separate the organic layer, and the organic layer was further washed three times with 100 mL of deionized water. After washing, the organic layer was dried over anhydrous magnesium sulfate. Thereafter, the insoluble matter was separated by filtration, and the filtrate was concentrated and dried by a rotary evaporator to obtain the compound (7-c). The obtained amount was 6.40 g (yield: 75%). A measurement result of an ¹H-NMR spectrum of the compound (7-c) is as follows.

¹H-NMR (400 MHz, CD₃COCD₃): δ(ppm)=9.85 (s, 1H), 7.81, 7.55 (ss, 1H), 7.17, 6.95 (ss, 1H), 2.03-1.87 (m, 4H), 1.50-1.17 (m, 16H), 0.94-0.77 (m, 6H).

Synthesis of Compound (7-c)

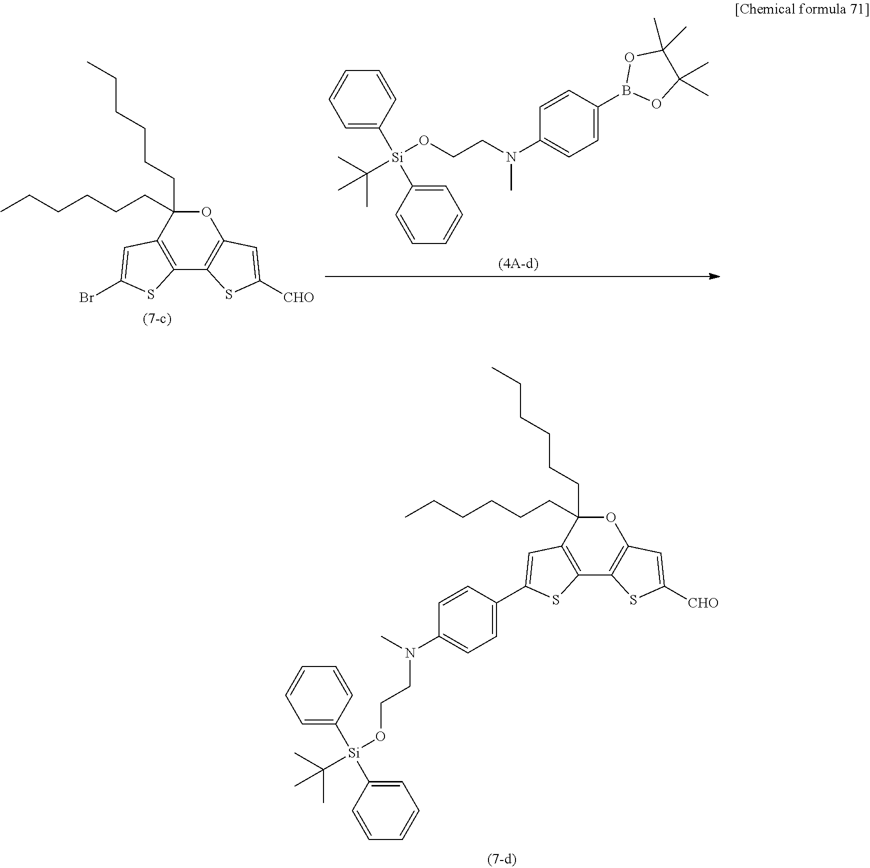 [SMILES: CCCCCCC1(CCCCCC)Oc2cc(C=O)sc2-c2sc(Br)cc21 ; CN(CCO[Si](c1ccccc1)(c1ccccc1)C(C)(C)C)c1ccc(B2OC(C)(C)C(C)(C)O2)cc1 ; CCCCCCC1(CCCCCC)Oc2cc(C=O)sc2-c2sc(-c3ccc(N(C)CCO[Si](c4ccccc4)(c4ccccc4)C(C)(C)C)cc3)cc21]

The compound (7-d) was synthesized from the compound (7-c) by Suzuki coupling. Into a 500 mL three-necked flask equipped with a three-way cock at a top and an induction type stirring blade, 3.00 g (6.39 mmol) of the compound (7-c), 4.94 g (9.58 mmol) of the compound (4A-d), and 200 mL of dehydrated THF were put, and the inside was replaced with argon. 0.18 g (0.13 mmol) of (tris(dibenzylideneacetone) dipalladium (0) (manufactured by Strem Chemicals) and 0.22 g (0.51 mmol) of tri-tert-butylphosphonium tetrafluoroborate (manufactured by Tokyo Chemical Industry Co., Ltd.) were added thereto while the reaction mixture was stirred, and 48 mL (144 mmol) of a 3 M potassium phosphate aqueous solution was further added thereto. The flask was immersed in an oil bath at 80° C., and reaction was caused for nine hours under reflux while the reaction mixture was vigorously stirred. After completion of the reaction, the reaction mixture was cooled to room temperature (25° C.), stirring was stopped, and the reaction mixture was allowed to stand still. The aqueous layer of the reaction mixture separated into two layers was removed, and the organic layer was dried over anhydrous magnesium sulfate. The insoluble matter was filtered. The filtrate was transferred to a 500 mL eggplant flask and concentrated by a rotary evaporator. The obtained concentrate was purified by column chromatography using toluene as an eluent to obtain the target compound (7-d). The obtained amount was 3.85 g (yield: 77%). A measurement result of an ¹H-NMR spectrum of the compound (7-d) is as follows.

¹H-NMR (400 MHz, CD₃COCD₃): δ(ppm)=9.82 (s, 1H), 7.73-7.64 (m, 4H), 7.55-7.35 (m, 8H), 6.75-6.68 (m, 2H), 3.86 (t, 2H), 3.65 (t, 2H), 2.81 (s, 3H), 2.03-1.87 (m, 4H), 1.50-1.17 (m, 16H), 1.03 (s, 9H), 0.94-0.77 (m, 6H).

Synthesis of Compound (7)

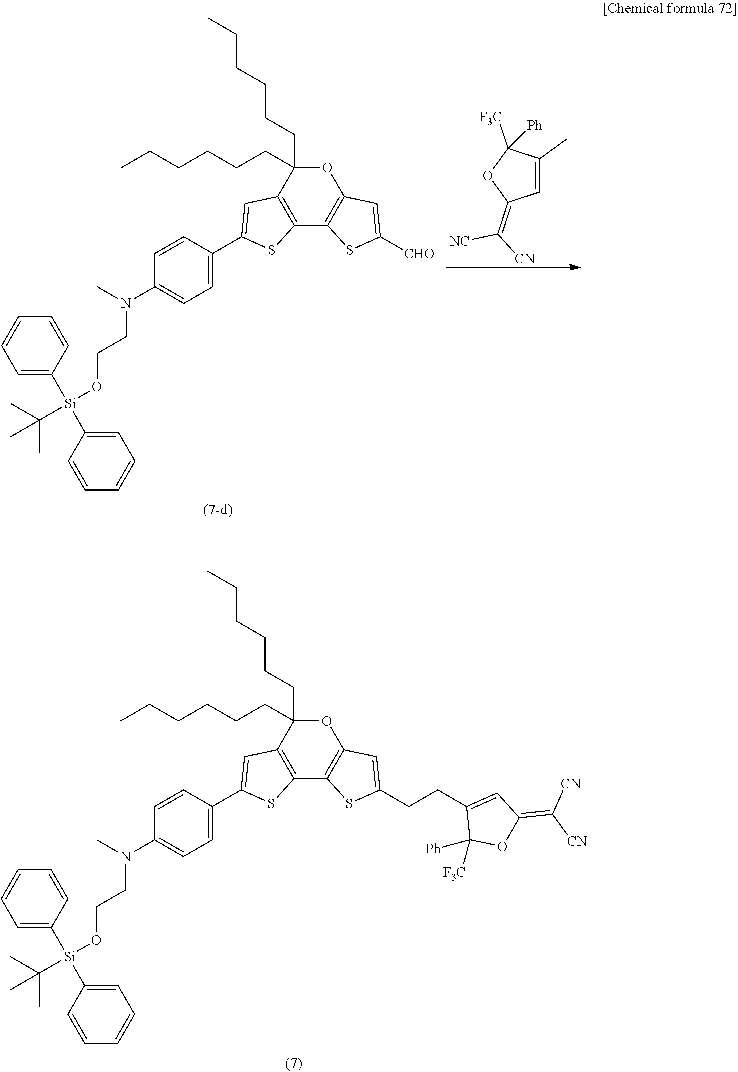 [SMILES: CCCCCCC1(CCCCCC)Oc2cc(C=O)sc2-c2sc(-c3ccc(N(C)CCO[Si](c4ccccc4)(c4ccccc4)C(C)(C)C)cc3)cc21 ; CC1=CC(=C(C#N)C#N)OC1(c1ccccc1)C(F)(F)F ; CCCCCCC1(CCCCCC)Oc2cc(CCC3=CC(=C(C#N)C#N)OC3(c3ccccc3)C(F)(F)F)sc2-c2sc(-c3ccc(N(C)CCO[Si](c4ccccc4)(c4ccccc4)C(C)(C)C)cc3)cc21]

The compound (7) was synthesized from the compound (7-d) by aldol condensation. Into a 500 mL eggplant flask equipped with a three-way cock at a top, 3.85 g (4.95 mmol) of the compound (7-d), 1.87 g (5.94 mmol) of 2-(3-cyano-4-methyl-5-phenyl-5-(trifluoromethyl)-2(5H)-furanylidene)-propanedinitrile (manufactured by iChemical), 100 mL of dehydrated ethanol, and 100 mL of dehydrated chloroform were put. A stirrer was further put thereinto, and the inside was replaced with nitrogen. Reaction was caused at room temperature (25° C.) for 24 hours while the reaction mixture was stirred with a magnetic stirrer. After completion of the reaction, the reaction mixture was concentrated by a rotary evaporator. Ethanol was added to the concentrate, the precipitate was separated by filtration, and the precipitate was further washed with methanol to obtain the compound (7) as a blue solid. The obtained amount was 4.55 g (yield: 86%). The compound (7) has a divalent polycyclic condensed ring group having two or more thiophene rings and having an sp3 carbon atom as a constituent element, and further has a group represented by formula (b1). Measurement results of an ¹H-NMR spectrum and a UV visible light spectrum of the compound (7) are as follows.

¹H-NMR (400 MHz, CD₃COCD₃): δ(ppm)=7.86-7.34 (m, 18H), 6.85-6.69 (m, 3H), 3.86 (t, 2H), 3.65 (t, 2H), 2.81 (s, 3H), 2.03-1.87 (m, 4H), 1.50-1.17 (m, 16H), 1.03 (s, 9H), 0.94-0.77 (m, 6H).

UV visible light spectrum: λmax=835 nm (in chloroform)

### (Example 1-8) Synthesis of Compound (8)

In order to synthesize a compound (8), a compound (8-a) was arylated to synthesize a compound (8-b). Subsequently, the compound (8-b) was ring-condensed to synthesize a compound (8-c). Subsequently, the compound (8-c) was formylated to synthesize a compound (8-d), and the compound (8-d) was brominated to synthesize a compound (8-e). Subsequently, a compound (8-f) was synthesized from the compound (8-e) by Suzuki coupling, and the compound (8) was synthesized from the compound (8-f) by aldol condensation.

Synthesis of Compound (8-b)

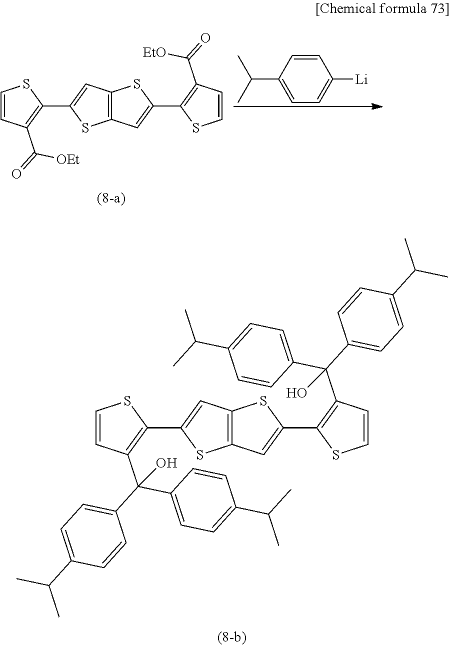

The compound (8-b) was synthesized by arylation of the compound (8-a). The compound (8-a) was synthesized by a method described in J. Mater. Chem. C, 2016, 4, 9656-9663. Into a 200 mL eggplant flask equipped with a three-way cock and a gas introduction tube, 8.43 g (42.4 mmol) of 4-bromocumene (manufactured by Aldrich) and 38 mL of dehydrated tetrahydrofuran (manufactured by KANTO CHEMICAL CO., INC.) were put. A stirrer was further put thereinto, the inside was replaced with nitrogen, and the solution was cooled to −60° C. 26 mL (41.5 mmol) of a 1.6 M n-butyllithium-hexane solution (manufactured by KANTO CHEMICAL CO., INC.) was added dropwise thereto, and then the resulting mixture was caused to react at −60° C. for 30 minutes while being stirred with a magnetic stirrer. 3.80 g (8.47 mmol) of the compound (8-a) was added to the reaction solution, and then the temperature was raised to 0° C. over one hour. 57 mL of methanol was added to the reaction mixture. Thereafter, the insoluble matter was separated by filtration, and the filtrate was concentrated by a rotary evaporator to obtain the compound (8-b) as a pale yellow solid. The obtained amount was 5.51 g (yield: 78%). A measurement result of an ¹H-NMR spectrum of the compound (8-b) is as follows.

¹H-NMR (400 MHz, CDCl₃): δ(ppm)=7.17-7.09 (m, 18H), 6.57 (s, 2H), 6.50 (d, 2H), 3.18 (s, 2H), 2.87 (sept, 4H), 1.22 (d, 24H).

Synthesis of Compound (8-c)

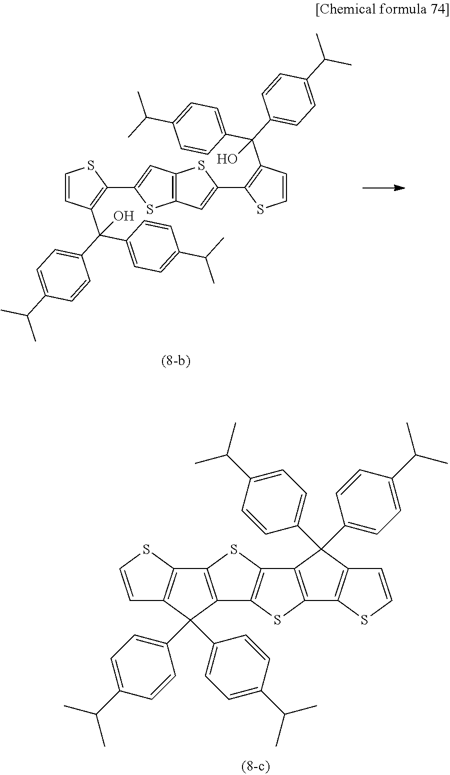

The compound (8-c) was synthesized by ring-condensation of the compound (8-b). Into a 500 mL eggplant flask equipped with a Dimroth with a three-way cock at a top and a gas introduction tube, 6.08 g (7.17 mmol) of the synthesized compound (8-b) and 240 mL of toluene (manufactured by KANTO CHEMICAL CO., INC.) were put. A stirrer was further put thereinto, and the inside was purged with nitrogen. 1.53 g (10.8 mmol) of a boron trifluoride-diethyl ether complex was added thereto, and the resulting mixture was caused to react under reflux condition for one hour while being stirred with a magnetic stirrer. The obtained reaction mixture was washed with deionized water. After washing, the organic layer was dried over anhydrous magnesium sulfate. Thereafter, the insoluble matter was separated by filtration, and the filtrate was concentrated by a rotary evaporator. The concentrate was washed with acetonitrile to obtain the compound (8-c) as a red solid. The obtained amount was 4.93 g (yield: 86%). A measurement result of an ¹H-NMR spectrum of the compound (8-c) is as follows.

¹H-NMR (400 MHz, CDCl₃): δ(ppm)=7.18-7.08 (m, 18H), 7.07 (s, 2H), 2.84 (sept, 4H), 1.25 (d, 24H).

Synthesis of Compound (8-d)

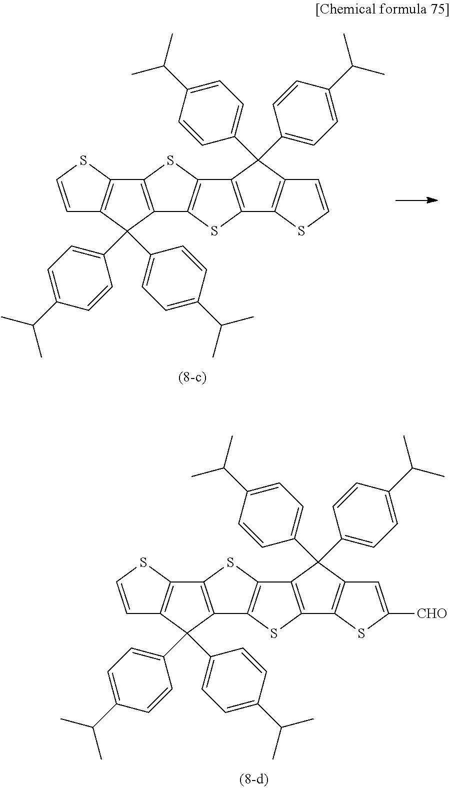

The compound (8-d) was synthesized by formylation of the compound (8-c). Into a 500 mL eggplant flask equipped with a Dimroth with a three-way cock at a top and a gas introduction tube, 4.76 g (5.94 mmol) of the synthesized compound (8-c), 71 mL of dehydrated tetrahydrofuran (manufactured by KANTO CHEMICAL CO., INC.), and 71 mL of dehydrated dimethylformamide (manufactured by KANTO CHEMICAL CO., INC.) were put. A stirrer was further put thereinto, and the inside was replaced with nitrogen. 1.14 g (8.91 mmol) of (chloromethylene) dimethyliminium chloride (manufactured by Tokyo Chemical Industry Co., Ltd.) was added thereto, and the resulting mixture was caused to react at 25° C. for nine hours while being stirred with a magnetic stirrer. 238 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain the compound (8-d) as a brown solid. The obtained amount was 5.21 g (yield: 105%).

Synthesis of Compound (8-e)

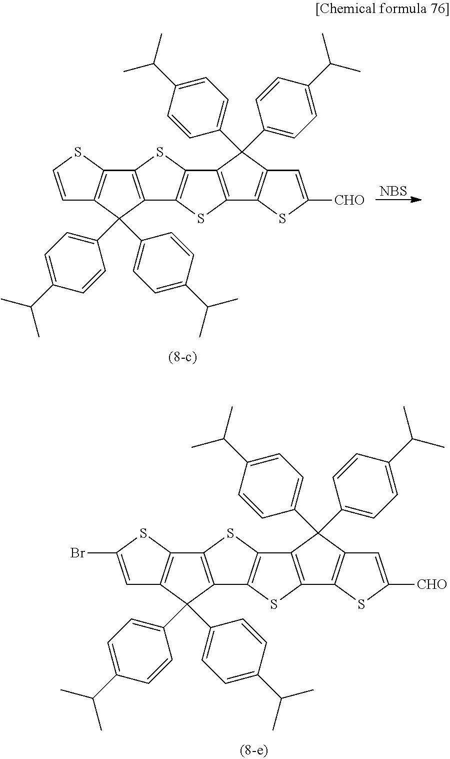

The compound (8-e) was synthesized by formylation of the compound (8-d). Into a 200 mL eggplant flask equipped with a three-way cock and a gas introduction tube, 5.20 g (5.16 mmol) of the synthesized compound (8-d) and 78 mL of dehydrated tetrahydrofuran (manufactured by FUJIFILM Wako Pure Chemical Corporation) were put. A stirrer was further put thereinto, the inside was replaced with nitrogen, and the solution was cooled to 0° C. 22.7 g (128 mmol) of N-bromosuccinimide (manufactured by Tokyo Chemical Industry Co., Ltd.) was added thereto, and then the resulting mixture was caused to react at 0° C. for one hour while being stirred with a magnetic stirrer. 47 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was purified with a silica gel column (toluene/ethyl acetate=100/0 to 90/10), and then washed with methanol to obtain the compound (8-e) as a red solid. The obtained amount was 2.20 g (yield: 47%). A measurement result of an ¹H-NMR spectrum of the compound (8-e) is as follows.

¹H-NMR (400 MHz, CDCl₃): δ(ppm)=9.76 (s, 1H), 7.65 (s, 1H), 7.17-7.10 (m, 16H), 7.08 (s, 1H), 7.07 (s, 2H), 2.86 (sept, 4H), 1.21 (d, 24H)

Synthesis of Compound (8-f)

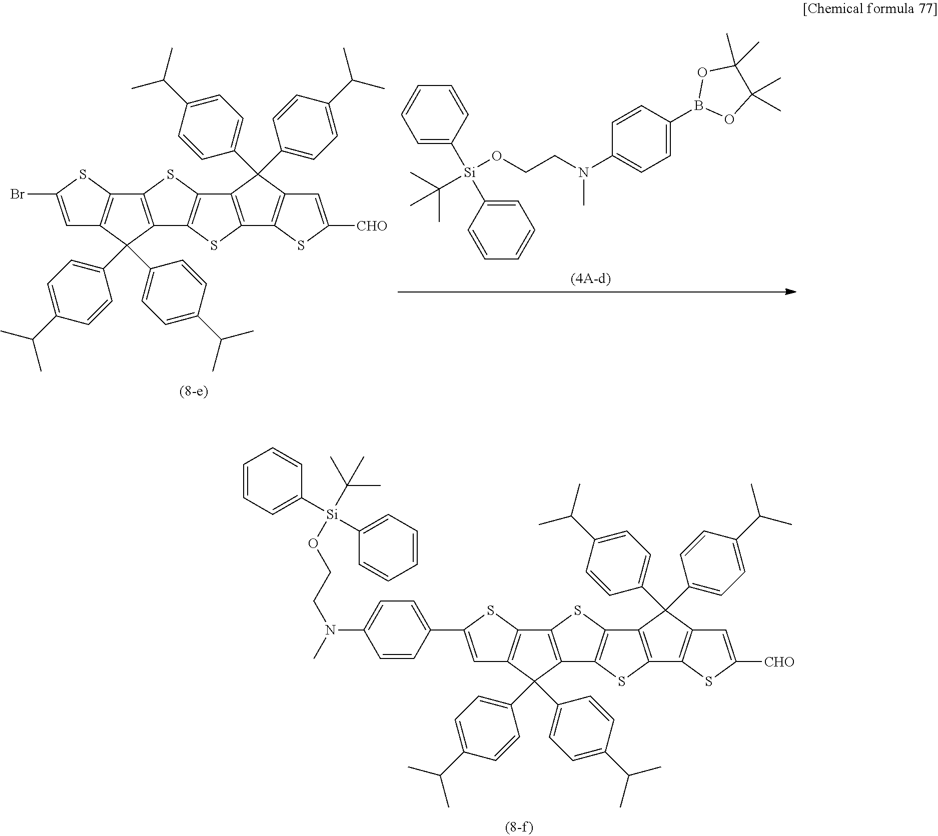

The compound (8-f) was synthesized from the compound (8-e) by Suzuki coupling. Into a 300 mL four-necked flask equipped with a Dimroth with a three-way cock at a top and a gas introduction tube, 0.55 g (0.96 mmol) of the synthesized compound (8-e), 0.59 g (1.15 mmol) of the compound (4A-d), and 90 mL of dehydrated tetrahydrofuran (manufactured by FUJIFILM Wako Pure Chemical Corporation) were put. A stirrer was further put thereinto, and the inside was replaced with nitrogen. 0.03 g (0.024 mmol) of tris(dibenzylideneacetone) dipalladium (0) (manufactured by N.E. CHEMCAT CORPORATION), 0.03 g (0.096 mmol) of tri-tert-butylphosphonium tetrafluoroborate (manufactured by FUJIFILM Wako Pure Chemical Corporation), and 1.3 mL of a 3 M potassium phosphate aqueous solution purged with nitrogen gas were put, and the resulting mixture was heated and stirred in an oil bath at 50° C. for 1.5 hours to be caused to react. 28 mL of toluene was added to the reaction mixture, and the organic layer was washed with deionized water. After washing, the organic layer was dried over anhydrous magnesium sulfate. Thereafter, the insoluble matter was separated by filtration, and the filtrate was concentrated by a rotary evaporator to obtain a crude product. The obtained crude product was washed with acetonitrile to obtain the compound (8-f) as a red solid. The obtained amount was 0.70 g (yield: 101%).

Synthesis of Compound (8)

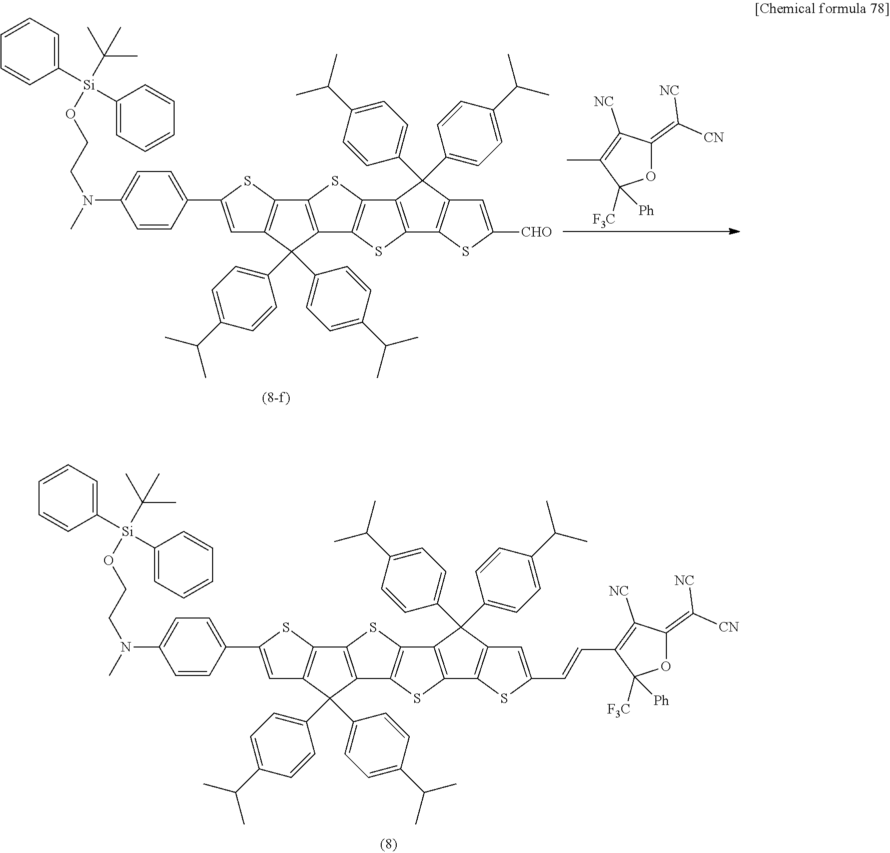 [SMILES: CC(C)c1ccc(C2(c3ccc(C(C)C)cc3)c3cc(C=O)sc3-c3sc4c5c(sc4c32)-c2sc(-c3ccc(N(C)CCO[Si](c4ccccc4)(c4ccccc4)C(C)(C)C)cc3)cc2C5(c2ccc(C(C)C)cc2)c2ccc(C(C)C)cc2)cc1 ; CC1=C(C#N)C(=C(C#N)C#N)OC1(c1ccccc1)C(F)(F)F ; CC(C)c1ccc(C2(c3ccc(C(C)C)cc3)c3cc(/C=C/C4=C(C#N)C(=C(C#N)C#N)OC4(c4ccccc4)C(F)(F)F)sc3-c3sc4c5c(sc4c32)-c2sc(-c3ccc(N(C)CCO[Si](c4ccccc4)(c4ccccc4)C(C)(C)C)cc3)cc2C5(c2ccc(C(C)C)cc2)c2ccc(C(C)C)cc2)cc1]

The compound (8) was synthesized from the compound (8-f) by aldol condensation. Into a 100 mL eggplant flask equipped with a three-way cock and a gas introduction tube, 0.54 g (0.44 mmol) of the synthesized compound (8-f), 0.34 g (1.07 mmol) of 2-(3-cyano-4-methyl-5-phenyl-5-(trifluoromethyl)-2(5H)-furanylidene)-propanedinitrile (manufactured by iChemical), 11 mL of dehydrated ethanol, and 11 mL of dehydrated chloroform were put. A stirrer was further put thereinto, and the inside was replaced with nitrogen. Reaction was caused at room temperature (25° C.) for 24 hours while the reaction mixture was stirred with a magnetic stirrer. After completion of the reaction, the reaction mixture was concentrated by a rotary evaporator to obtain a crude product. The crude product was purified with a reversed-phase silica gel column (acetonitrile/ethyl acetate=80/20) and then washed with ethyl acetate and hexane to obtain the compound (8) as a green solid. The obtained amount was 0.36 g (yield: 54%). The compound (8) has a divalent polycyclic condensed ring group having two or more thiophene rings and having an sp3 carbon atom as a constituent element, and further has a group represented by formula (b1). Measurement results of an ¹H-NMR spectrum and a UV visible light spectrum of the compound (8) are as follows.

¹H-NMR (400 MHz, CDCl₃): δ(ppm)=7.61 (dd, 4H), 7.55-7.47 (m, 5H), 7.44-7.31 (m, 9H), 6.90 (s, 1H), 6.77 (s, 1H), 6.59-6.53 (m, 3H), 3.80 (t, 2H), 3.53 (t, 2H), 3.00 (s, 3H), 1.84-1.76 (m, 6H), 1.03 (s, 9H), 0.85 (d, 6H), 0.83 (d, 6H).

UV visible light spectrum: λmax=879 nm (in chloroform)

2. Evaluation of Heat Resistance of Compound

### (Example 2-1) Evaluation of Compound (1)

A heat treatment was performed using a thermogravimeter-differential thermal analyzer (TG-DTA). 1.52 mg of the compound (1) was placed on an aluminum pan. The temperature was raised from 25° C. at a rate of 10° C./min under a nitrogen atmosphere, and was maintained for five minutes after reaching 200° C. After cooling, the compound (1) was put into a screw tube together with the aluminum pan and eluted into chloroform (super dehydrated) to adjust a solution. A UV visible light spectrum of the prepared solution was measured, and a maximum absorption wavelength λmax before the heat treatment was compared with that after the heat treatment. It can be said that the smaller the amount of change in maximum absorption wavelength λmax before and after the heat treatment, the higher the heat resistance of the compound. λmax before the heat treatment was 736 nm, whereas λmax after the heat treatment was 734 nm.

### (Example 2-2) Evaluation of Compound (2)

A heat treatment was performed in a similar manner to Example 2-1 except that 2.00 mg of the compound (2) was used instead of the compound (1), and a UV visible light spectrum was measured. λmax before the heat treatment was 656 nm, whereas λmax after the heat treatment was 655 nm.

### (Example 2-3) Evaluation of Compound (3)

A heat treatment was performed in a similar manner to Example 2-1 except that 1.62 mg of the compound (3) was used instead of the compound (1), and a UV visible light spectrum was measured. λmax before the heat treatment was 649 nm, whereas λmax after the heat treatment was 648 nm.

### (Example 2-4) Evaluation of Compound (4)

A heat treatment was performed in a similar manner to Example 2-1 except that 1.68 mg of the compound (4) was used instead of the compound (1), and a UV visible light spectrum was measured. λmax before the heat treatment was 794 nm, whereas λmax after the heat treatment was 790 nm.

### (Example 2-5) Evaluation of Compound (5)

A heat treatment was performed in a similar manner to Example 2-1 except that 1.73 mg of the compound (5) was used instead of the compound (1), and a UV visible light spectrum was measured. λmax before the heat treatment was 824 nm, whereas λmax after the heat treatment was 807 nm.

### (Example 2-6) Evaluation of Compound (6)

A heat treatment was performed in a similar manner to Example 2-1 except that 1.92 mg of the compound (6) was used instead of the compound (1), and a UV visible light spectrum was measured. λmax before the heat treatment was 833 nm, whereas λmax after the heat treatment was 831 nm.

### (Example 2-7) Evaluation of Compound (7)

A heat treatment was performed in a similar manner to Example 2-1 except that 1.96 mg of the compound (7) was used instead of the compound (1), and a UV visible light spectrum was measured. λmax before the heat treatment was 835 nm, whereas λmax after the heat treatment was 830 nm.

### (Example 2-8) Evaluation of Compound (8)

A heat treatment was performed in a similar manner to Example 2-1 except that 1.84 mg of the compound (8) was used instead of the compound (1), and a UV visible light spectrum was measured. λmax before the heat treatment was 879 nm, whereas λmax after the heat treatment was 878 nm.

### (Comparative Example 2-1) Evaluation of Compound NEO-823

A heat treatment was performed in a similar manner to Example 2-1 except that 1.80 mg of a commercially available compound NEO-823 (manufactured by Tokyo Chemical Industry Co., Ltd.) was used instead of the compound (1), and a UV visible light spectrum was measured. Note that the compound NEO-823 does not have a divalent polycyclic condensed ring group having two or more thiophene rings and having an sp3 carbon atom as a constituent element. λmax before the heat treatment was 820 nm, whereas λmax after the heat treatment was 704 nm.

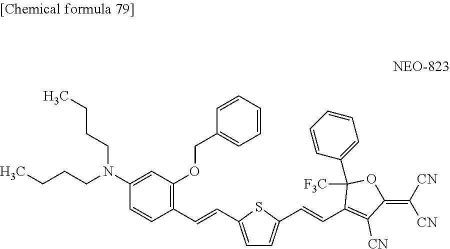 [SMILES: CCCCN(CCCC)c1ccc(/C=C/c2ccc(/C=C/C3=C(C#N)C(=C(C#N)C#N)OC3(c3ccccc3)C(F)(F)F)s2)c(OCc2ccccc2)c1]

TABLE 1

| | | | | Amount of |
|---|---|---|---|---|
| | | | | change in |
| | | | | λmax (nm) |
| | | λmax (nm) | λmax (nm) | before and |
| | | before heat | after heat | after heat |
| | Compound | treatment | treatment | treatment |
| Example 2-1 | Compound (1) | 736 | 734 | −2 |
| Example 2-2 | Compound (2) | 656 | 655 | −1 |
| Example 2-3 | Compound (3) | 649 | 648 | −1 |
| Example 2-4 | Compound (4) | 794 | 790 | −4 |
| Example 2-5 | Compound (5) | 824 | 807 | −17 |
| Example 2-6 | Compound (6) | 833 | 831 | −2 |
| Example 2-7 | Compound (7) | 835 | 830 | −5 |
| Example 2-8 | Compound (8) | 879 | 878 | −1 |
| Comparative | Compound | 820 | 704 | −116 |
| Example 2-1 | NEO-823 | | | |

As presented in Table 1, it was found that each of the compounds of Examples 2-1 to 2-8 had an extremely small amount of change in λmax before and after the heat treatment as compared with the compound of Comparative Example 2-1. From this, it was confirmed that the compound of the present invention had excellent heat resistance.

3. Calculation of Hyperpolarizability of Compound

### Examples 3-1 to 3-7 and Comparative Example 3-1

A hyperpolarizability B, which is an index of EO characteristics, was calculated for the compounds (1) to (3) and (5) to (8) and the compound NEO-823. As a reference, the hyperpolarizability B was also calculated for a compound R3 having an equivalent acceptor site. Note that the compound R3 does not have a divalent polycyclic condensed ring group having two or more thiophene rings and having an sp3 carbon atom as a constituent element. The calculated hyperpolarizability β was calculated by Gaussian 16 which is a quantum chemical calculation program manufactured by Gaussian. Structure optimization calculation was performed by pcm calculation (specifying chloroform as a solvent) under an M062X/6-31+g(d) condition. Furthermore, the hyperpolarizability β was calculated by adding a keyword (polar=enonly) of polarizability calculation to the same calculation conditions as those of the structure optimization calculation for the optimized structure. Note that the calculation was performed using a structure in which all alkyl side chains were replaced with methyl groups as a model for reducing a calculation load. Results thereof are presented in Table 2.

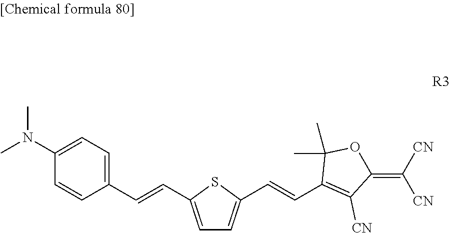 [SMILES: CN(C)c1ccc(/C=C/c2ccc(/C=C/C3=C(C#N)C(=C(C#N)C#N)OC3(C)C)s2)cc1]

TABLE 2

| | | Hyperpolarizability |
|---|---|---|
| | EO compound | β (a.u.) |
| Example 3-1 | Compound (1) | 1702 |
| Example 3-2 | Compound (2) | 1648 |
| Example 3-3 | Compound (3) | 1419 |
| Example 3-4 | Compound (5) | 2439 |
| Example 3-5 | Compound (6) | 2439 |
| Example 3-6 | Compound (7) | 2439 |
| Example 3-7 | Compound (8) | 3589 |
| Comparative | Compound R3 | 1601 |
| Example 3-1 | | |

As presented in Table 2, numerical values of the hyperpolarizability β of the compounds (1) to (3) and (5) to (8) calculated by the calculation were all high. These results suggest that the compound of the invention has a high hyperpolarizability.

4. Measurement of EO Coefficient r33 of EO Film

### Examples 4-1 to 4-3 and Comparative Example 4-1

An EO coefficient r33 of an EO film was measured using the compounds (6) to (8) and the compound NEO-823.

<Production of EO Film>

Each of the above compounds and polymethyl methacrylate (PMMA) were adjusted to have a mass ratio of 2:8, and dissolved in o-dichlorobenzene or chlorobenzene to prepare a coating solution. The coating solution was applied to a washed substrate (ITO-coated glass, quartz glass) at a speed of 500 to 3000 rpm using a spin coater MS-A100 (manufactured by Mikasa Co., Ltd.), and then vacuum-dried for one hour around a glass transition temperature (Tg). Conditions of the type of the solvent, the concentration of the solution, and a rotation speed of the spin coater were appropriately selected so as to obtain a desired film thickness, and each EO film was produced.

<Measurement of EO Coefficient of EO Film>

Using the obtained film, the EO coefficient r33 of the EO film was measured in a similar manner to a method described in a reference paper (“Transmission ellipsometric method without an aperture for simple and reliable evaluation of electro-optic properties”, Toshiki Yamada and Akira Otomo, Optics Express, voI. 21, pages 29240-48 (2013)). As laser light sources, LP1310-SAD2 (1310 nm) and LP1550-SAD2 (1550 nm) of semiconductor DFB laser (manufactured by THORLABS) were used. Results thereof are presented in Table 3.

TABLE 3

| | | | Poling | | |
|---|---|---|---|---|---|
| | | Film | electric | r33 (1310 | r33 (1550 |
| | | thickness | field | nm) | nm) |
| | Compound | (nm) | (V/μm) | (pm/V) | (pm/V) |
| Example 4-1 | Compound | 880 | 80 | 48 | 35 |
| | (6) | | | | |
| Example 4-2 | Compound | 1054 | 80 | 42 | 29 |
| | (7) | | | | |
| Example 4-3 | Compound | 880 | 80 | 49 | 32 |
| | (8) | | | | |
| Comparative | Compound | 1264 | 80 | 62 | 45 |
| Example 4-1 | NEO-823 | | | | |

As presented in Table 3, numerical values of the EO coefficient r33 of the EO films containing the compounds (6) to (8) were all sufficiently high. From these results, it was found that the compound of the present invention had excellent heat resistance and exhibited a high EO coefficient when being used in an EO element.

1. A compound represented by the following formula (1). 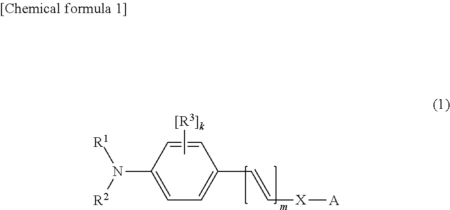
[In formula (1), X represents a divalent polycyclic condensed ring group having two or more thiophene rings and having at least one selected from the group consisting of an sp3 carbon atom, a nitrogen atom, and a silicon atom as a constituent element, and the divalent polycyclic condensed ring group may have a substituent.
R¹ and R² each independently represent an alkyl group, a haloalkyl group, an acyloxyalkyl group, a silyloxyalkyl group, an aryl group, —R²¹—OH (R²¹ represents a divalent hydrocarbon group), —R²²—NH₂ (R²² represents a divalent hydrocarbon group), —R²³—SH (R²³ represents a divalent hydrocarbon group), or —R²⁴—NCO (R²⁴ represents a divalent hydrocarbon group). These groups may each have a crosslinkable group. R¹ and R² may be bonded to each other to form a ring together with atoms to which R¹ and R² are bonded.
R³ represents an alkyl group, an alkyloxy group, an aryl group, an aryloxy group, an aralkyloxy group, a silyloxyalkyl group, an alkenyloxy group, an alkynyloxy group, a hydroxy group, an amino group, a sulfanyl group, an isocyanate group, —R³¹—OH (R³¹ represents a divalent hydrocarbon group), —O—R³²—OH (R³² represents a divalent hydrocarbon group), —R³³—NH₂ (R³³ represents a divalent hydrocarbon group), —R³⁴—SH (R³⁴ represents a divalent hydrocarbon group), —R³⁵—NCO (R³⁵ represents a divalent hydrocarbon group), or —OC(═O)R⁴¹ (R⁴¹ represents a monovalent hydrocarbon group). These groups may each have a crosslinkable group. When there is a plurality of R³s, R's may be the same as or different from each other. R³ may be bonded to R¹ or R² to form a ring together with atoms to which R³ and R¹ or R² are bonded.
k represents an integer of 0 to 4.
A represents any one of groups represented by the following formulas (a1), (a2), and (b1). 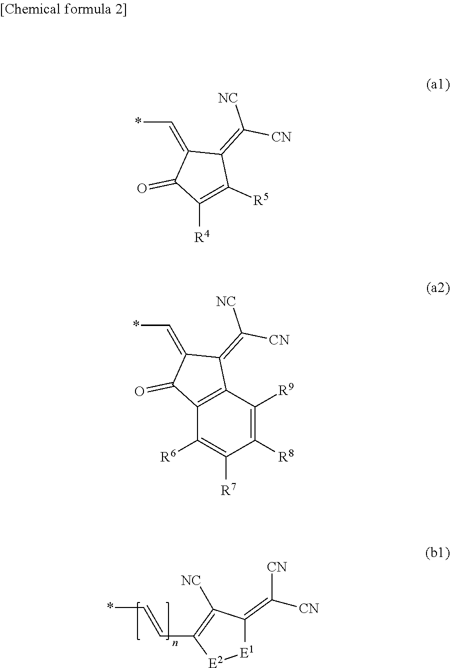
R⁴, R⁵, R⁶, R⁷, R⁸, and R⁹ each independently represent a hydrogen atom, a halogen atom, an alkyl group, a haloalkyl group, a cyano group, an aryl group, or a haloaryl group.
E¹ and E² each independently represent —C(R¹⁰)(R¹¹)—, —C(O)—, —O—, or —NR¹²—. Note that at least one of E¹ and E² is —O— or —NR¹²—. R¹⁰ and R¹¹ each independently represent a hydrogen atom, an alkyl group, a haloalkyl group, an aryl group, or a haloaryl group. R¹² represents a hydrogen atom or an alkyl group.
When A is a group represented by formula (a1) or (a2), m is 0.
When A is a group represented by formula (b1), m and n each independently represent 0 or 1. Note that m+n=1.]

2. The compound according to claim 1, wherein the compound represented by the formula (1) is a compound represented by the following formula (2a2) or (2b1). 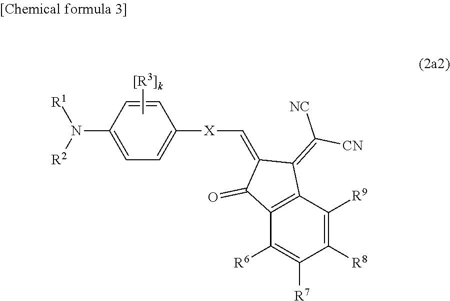
[In formula (2a2), X, R¹, R², R³, R⁶, R⁷, R⁸, R⁹, and k have the same meanings as those described above.] 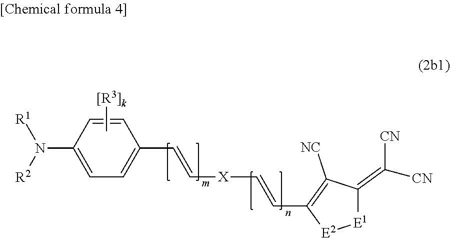
[In formula (2b1), X, R¹, R², R³, E¹, E², k, m, and n have the same meanings as those described above.]

3. The compound according to claim 1, wherein the compound represented by the formula (1) is a compound represented by the following formula (2b1-1). 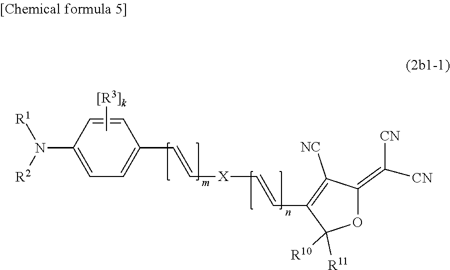
[In formula (2b1-1), X, R¹, R², R³, k, m, and n have the same meanings as those described above.
R¹⁰ and R¹¹ each independently represent a methyl group, a trifluoromethyl group, a phenyl group, or a pentafluorophenyl group.]

4. An electrooptic composition comprising the compound according to claim 1.

5. An electrooptic film comprising the compound according to claim 1.

6. An electrooptic element comprising the electrooptic film according to claim 5.
